(19) United States
(12) Patent Application Publication
(10) Pub. No.: US 2021/0330760 A1
(43) Pub. Date: Oct. 28, 2021

(54) METHODS AND MATERIALS FOR TREATING OSTEOARTHRITIS

(71) Applicant: University of Pittsburgh - Of the Commonwealth System of Higher Education, Pittsburgh, PA (US)

(72) Inventors: Fabrisia Ambrosio, Pittsburgh, PA (US); Hirotaka Iijima, Pittsburgh, PA (US)

(21) Appl. No.: 17/236,726

(22) Filed: Apr. 21, 2021

Related U.S. Application Data
(60) Provisional application No. 63/013,891, filed on Apr. 22, 2020.
(60) Provisional application No. 63/163,641, filed on Mar. 19, 2021.

(51) Int. Cl. A61K 38/47; A61P 19/02; A61K 9/00; C12N 15/86; C12N 7/00

(52) U.S. Cl. CPC A61K 38/47; A61P 19/02; C12Y 302/01031; A61K 48/00; C12N 15/86; C12N 7/00; A61K 9/0019

(57) ABSTRACT

This document provides methods and materials for treating osteoarthritis. For example, a mammal having, or at risk for developing, osteoarthritis can be treated by increasing the level of one or more Klotho polypeptides (e.g., one or more alpha-Klotho (α-Klotho) polypeptides) within cells (e.g., chondrocytes) within the mammal.

### CROSS-REFERENCE TO RELATED APPLICATIONS

This application claims the benefit of U.S. Patent Application Ser. No. 63/013,891, filed on Apr. 22, 2020, and claims the benefit of U.S. Patent Application Ser. No. 63/163,641, filed on Mar. 19, 2021. The disclosures of the prior applications are considered part of (and are incorporated by reference in) the disclosure of this application.

### BACKGROUND

### 1. Technical Field

This document relates to methods and materials for treating osteoarthritis. For example, a mammal having, or at risk for developing, osteoarthritis can be treated by increasing the level of one or more Klotho polypeptides (e.g., one or more alpha-Klotho (α-Klotho) polypeptides) within cells (e.g., chondrocytes) within the mammal.

### 2. Background Information

Knee osteoarthritis is a degenerative articular cartilage disease and the leading cause of knee pain, disability, and deterioration of the quality of life worldwide (Guccione, American Journal of Public Health, 84(3):351-8 (1994); and Peat, Ann. Rheum. Dis., 60(2):91-7 (2001)). In 2010, knee osteoarthritis was the 11th leading cause of disability worldwide, with its incidence increasing in the last two decades (Vos, Lancet, 380(9859): 2197-223 (2012)).

### SUMMARY

According to clinical guidelines, non-pharmacological treatments such as patient education, weight management, and exercise are the first line strategies for the management of osteoarthritis (see, e.g., McAlindon, Osteoarthritis Cartilage, 22(3):363-88 (2014)). These non-pharmacological treatments have often been combined with pharmacological treatments, such as non-opioid oral analgesics (see, e.g., McAlindon, Osteoarthritis Cartilage, 22(3): 363-88 (2014)). These therapeutic strategies focus on reducing pain and improving physical function, and fail to address the underlying pathology. Developing an effective disease-modifying therapy, as opposed to simply a pain-relieving therapy, is a critical challenge for reducing the burden of knee osteoarthritis.

This document provides methods and materials for treating osteoarthritis. For example, a mammal having, or at risk for developing, osteoarthritis can be treated by increasing the level of one or more Klotho polypeptides (e.g., one or more α-Klotho polypeptides) within cells (e.g., chondrocytes) within the mammal. In some cases, one or more Klotho polypeptides (e.g., one or more α-Klotho polypeptides) can be administered to a mammal having, or at risk for developing, osteoarthritis (e.g., knee osteoarthritis) to treat the mammal. In some cases, nucleic acid encoding one or more Klotho polypeptides (e.g., one or more α-Klotho polypeptides) can be administered to a mammal having, or at risk for developing, osteoarthritis (e.g., knee osteoarthritis) to treat the mammal.

As demonstrated herein, exogenous delivery of α-Klotho polypeptides can improve cartilage integrity and can reduce cartilage degeneration. The ability to improve cartilage integrity and to reduce cartilage degeneration provides an opportunity to treat mammals having, or at risk for developing, osteoarthritis (e.g., knee osteoarthritis). For example, treating osteoarthritis by administering one or more α-Klotho polypeptides and/or nucleic acid encoding one or more α-Klotho polypeptides can reduce the effects of, reduce the severity of the symptoms of, or slow the progression of osteoarthritis in an efficient and cost-effective way.

In general, one aspect of this document features a method for treating a mammal having osteoarthritis. The method comprises (or consists essentially of or consists of) (a) identifying the mammal as having osteoarthritis, and (b) administering an α-Klotho polypeptide or nucleic acid encoding the α-Klotho polypeptide to the mammal. The mammal can be a human. The method can comprise administering the α-Klotho polypeptide to the mammal. The method can comprise administering the nucleic acid to the mammal. The nucleic acid can be a viral vector. The nucleic acid can be naked DNA. The osteoarthritis can be present in a knee of the mammal. The administering can comprise an intra-articular injection. The administering can comprise an intravenous injection.

In another aspect, this document features a method for treating a mammal having osteoarthritis. The method comprises (or consists essentially of or consists of) administering an α-Klotho polypeptide or nucleic acid encoding the α-Klotho polypeptide to a mammal identified as having osteoarthritis. The mammal can be a human. The method can comprise administering the α-Klotho polypeptide to the mammal. The method can comprise administering the nucleic acid to the mammal. The nucleic acid can be a viral vector. The nucleic acid can be naked DNA. The osteoarthritis can be present in a knee of the mammal. The administering can comprise an intra-articular injection. The administering can comprise an intravenous injection.

In another aspect, this document features a method for improving cartilage integrity within a mammal having osteoarthritis. The method comprises (or consists essentially of or consists of) (a) identifying the mammal as having osteoarthritis, and (b) administering an α-Klotho polypeptide or nucleic acid encoding the α-Klotho polypeptide to the mammal. The mammal can be a human. The method can comprise administering the α-Klotho polypeptide to the mammal. The method can comprise administering the nucleic acid to the mammal. The nucleic acid can be a viral vector. The nucleic acid can be naked DNA. The osteoarthritis can be present in a knee of the mammal. The administering can comprise an intra-articular injection. The administering can comprise an intravenous injection.

In another aspect, this document features a method for improving cartilage integrity within a mammal having osteoarthritis. The method comprises (or consists essentially of or consists of) administering an α-Klotho polypeptide or nucleic acid encoding the α-Klotho polypeptide to a mammal identified as having osteoarthritis. The mammal can be a human. The method can comprise administering the α-Klotho polypeptide to the mammal. The method can comprise administering the nucleic acid to the mammal. The nucleic acid can be a viral vector. The nucleic acid can be naked DNA. The osteoarthritis can be present in a knee of the mammal. The administering can comprise an intra-articular injection. The administering can comprise an intravenous injection.

In another aspect, this document features a method for reducing cartilage degeneration within a mammal having osteoarthritis. The method comprises (or consists essentially of or consists of) (a) identifying the mammal as having osteoarthritis, and (b) administering an α-Klotho polypeptide or nucleic acid encoding the α-Klotho polypeptide to the mammal. The mammal can be a human. The method can comprise administering the α-Klotho polypeptide to the mammal. The method can comprise administering the nucleic acid to the mammal. The nucleic acid can be a viral vector. The nucleic acid can be naked DNA. The osteoarthritis can be present in a knee of the mammal. The administering can comprise an intra-articular injection. The administering can comprise an intravenous injection.

In another aspect, this document features a method for reducing cartilage degeneration within a mammal having osteoarthritis. The method comprises (or consists essentially of or consists of) administering an α-Klotho polypeptide or nucleic acid encoding the α-Klotho polypeptide to a mammal identified as having osteoarthritis. The mammal can be a human. The method can comprise administering the α-Klotho polypeptide to the mammal. The method can comprise administering the nucleic acid to the mammal. The nucleic acid can be a viral vector. The nucleic acid can be naked DNA. The osteoarthritis can be present in a knee of the mammal. The administering can comprise an intra-articular injection. The administering can comprise an intravenous injection.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. An exemplary experimental flow diagram for evaluating young vs. aged mice. The knee joints of young (3-6 months old) and aged (21-24 months old) male mice were harvested. Knee joints were decalcified and embedded in paraffin. Safranin-O/Fast green staining and immunofluorescence were performed to evaluate: (i) cartilage degeneration, and (ii) α-Klotho expression level in chondrocytes. ImageJ and Matlab were used to perform quantification of irregularity of the knee cartilage surface, and NIS Elements software was used to quantify α-Klotho expression per cell.

FIGS. 2A-2C. Quantification of relative cartilage degeneration with age in mice. FIG. 2A) Representative histological images of knee cartilage indicating the Safranin-O staining. Scale: 50 μm. Quantification of cartilage degeneration using (FIG. 2B) Osteoarthritis Research Society International (OARSI) based scores and (FIG. 2C) modified Mankin's score. Two-tailed Student's t-test, n=5/group.

FIG. 3. Quantification of surface irregularity of cartilage in aging mice. Curve fitting was applied to cartilage surface in histological image using Matlab version R2019a. Surface irregularity was calculated as the integrated value of the distance between the fitted curve and entire actual cartilage surface. Two-tailed Student's t-test, n=5/group.

FIGS. 4A-4C. Quantification of relative abundance of Klotho protein in cartilage tissue. FIG. 4A) Representative immunofluorescence images of knee cartilage indicating the Klotho staining and DAPI. Scale: 10 FIG. 4B) Quantification of number of cells per frame of the cartilage image. FIG. 4C) Quantification of Klotho expression per cell in a frame of cartilage image. p<0.05, two-tailed Student's t-test, n=5/group.

FIG. 5. An exemplary experimental flow diagram for evaluating young vs. old human knees. The knee joint cartilage of young (15 years old) and old (69 years old) human sample were harvested. Cartilages were decalcified and embedded in paraffin. Safranin-O/Fast green staining and immunofluorescence were performed to evaluate: (i) the cartilage degeneration, and (ii) α-Klotho expression level in chondrocytes. ImageJ and Matlab were used to perform quantification of irregularity of the knee cartilage surface, and NIS Elements software was used to quantify α-Klotho expression per cell.

FIGS. 6A-6C. Quantification of relative cartilage degeneration with age in human sample. FIG. 6A) Representative histological images of knee cartilage indicating the Safranin-O staining. Scale: 100 μm. Quantification of cartilage degeneration using (FIG. 6B) OARSI based scores and (FIG. 6C) modified Mankin's score. n=1/group.

FIG. 7. Quantification of surface irregularity of cartilage in aging human. Curve fitting was applied to cartilage surface in histological image using Matlab version R2019a. Surface irregularity was calculated as the integrated value of the distance between the fitted curve and entire actual cartilage surface. n=1/group.

FIGS. 8A-8F. Quantification of relative abundance of Klotho protein in human cartilage tissue in superficial (FIGS. 8A-8C) and deep zones (FIGS. 8D-8F). FIGS. 8A and 8D) Representative immunofluorescence images of knee cartilage indicating the Klotho staining and DAPI. Scale: 10 FIGS. 8B and 8E) Quantification of number of cells per frame of the cartilage image. FIGS. 8C and 8F) Quantification of Klotho expression per cell in a frame of cartilage image. n=1/group.

FIG. 9. An exemplary experimental flow diagram for evaluating young α-Klotho^{+/-} mice. The knee joints of α-Klotho^{+/-} (3-4 months old) and wild type (3-4 months old) male mice were harvested. Decalcified paraffin sections stained with Safranin-O/Fast green were used for histological analysis.

FIG. 10A-10C. Quantification of relative cartilage degeneration in young α-Klotho mice. FIG. 10A) Representative histological images of knee cartilage indicating the Safranin-O staining. Scale: 50 μm. Quantification of cartilage degeneration using (FIG. 10B) OARSI based scores and (FIG. 10C) modified Mankin's score. p<0.05, two-tailed Student's t-test, n=5/group.

FIG. 11. Quantification of surface irregularity of cartilage in young α-Klotho^{+/-} mice. Curve fitting was applied to cartilage surface in histological image using Matlab version R2019a. Surface irregularity was calculated as the integrated value of the distance between the fitted curve and entire actual cartilage surface. p<0.05, two-tailed Student's t-test, n=5/group.

FIG. 12. An exemplary experimental flow diagram for evaluating aged α-Klotho^{+/-} mice. The knee joints of α-Klotho^{+/-} (21-24 months old) and wild type (21-24 months old) male mice were harvested. Decalcified paraffin sections stained with Safranin-O/Fast green were used for histological analysis.

FIGS. 13A-13C. Quantification of relative cartilage degeneration in aged α-Klotho^{+/-} mice. FIG. 13A) Representative histological images of knee cartilage indicating the Safranin-O staining. Scale: 50 μm. Quantification of cartilage degeneration using (FIG. 13B) OARSI based scores and (FIG. 13C) modified Mankin's score. n=3 for wild type group and n=5 for Klotho group.

FIG. 14. An exemplary experimental flow diagram for evaluating effects of AAV-delivered α-Klotho administration on knee joints. The knee joints of aged (21 months old) male mice treated with low-dose AAV Klotho, high-dose AAV Klotho, or a control (AAV GFP) were harvested. Decalcified paraffin sections stained with Safranin-O/Fast green were used for histological analysis.

FIGS. 15A-15C. Quantification of relative cartilage degeneration in aged mice with AAV Klotho at low-dose and high-dose. FIG. 15A) Representative histological images of knee cartilage indicating the Safranin-O staining. Scale: 50 μm. Quantification of cartilage degeneration using (FIG. 15B) OARSI based scores and (FIG. 15C) modified Mankin's score. (p<0.05, two-tailed Student's t-test, n=5/group.

FIGS. 16A-16D. Nuclear morphology of chondrocytes in cartilage tissue from young and aged mice. FIG. 16A) Representative images of nuclear shapes of chondrocytes in histological section of knee cartilage in ImageJ. Scale: 10 μm. Quantification of nuclear morphology in ImageJ defined by (FIG. 16B) nuclear aspect ratio, (FIG. 16C) nuclear circularity and (FIG. 16D) nuclear size. Two-tailed Student's t-test, n=5/group.

FIG. 17. An amino acid sequence (SEQ ID NO:1) of a human klotho precursor polypeptide.

FIG. 18. A nucleic acid sequence (SEQ ID NO:2) encoding a human klotho precursor polypeptide.

FIGS. 19A-19F. Aging induced progression cartilage degeneration and surface roughness in a sex-dependent manner. FIG. 19A) Schematic showing the experimental protocol. Human age equivalents is provided. FIG. 19B) Aging induces progressive cartilage degeneration in murine medial tibial plateaus in a sex-dependent manner. Representative histological sections stained with Safranin-O/Fast Green are provided in each group. Black arrow heads indicate loss of cartilage matrix. OARSI score (0-24 points; higher value indicates more severe cartilage degeneration) assessed by blinded assessor is provided. FIG. 19C) Computational analysis for calculation of cartilage surface roughness. Surface roughness was calculated as the deviation between actual cartilage surface (blue solid line) and fitted curve applied to the cartilage surface (red solid line). FIG. 19D) Representative error value between fitting curve and cartilage surface. FIG. 19E) Aging induces progressive surface roughness in murine medial tibial plateaus in a sex-dependent manner. FIG. 19F) Similar serum Estrogen level across three groups in female mice (n=4 in young, n=5 in middle-aged and aged). Statistical analysis was performed using linear regression analysis (FIGS. 19B, 19E, and 19F). Data are presented as means±95% confidence intervals.

FIGS. 20A-20F. Mass spectrometry proteomics reveal age-enrichment of PI3K-Akt signaling pathway in male mice but not female mice. FIG. 20A) Schematic showing the experimental protocol. Knee cartilage was micro-dissected from young, middle-aged, and aged male and female mice (n=5/sex/age). FIG. 20B) Individual proteins up- and downregulated with aging for male and female mice. FIG. 20C) Pathway analysis for young vs. aged in male and female mice. FIG. 20D) Comparison between young vs. aged and young vs. middle-aged analyses in male mice, and display of perturbation calculations. FIG. 20E) Heat map of detected proteins that are involved with PI3K/Akt Signaling. FIG. 20F) Schematic correlating FIG. 19 findings of cartilage degeneration to pathway involving PI3K/Akt Signaling and linking to α-Klotho.

FIGS. 21A-21C. Aging induced progressive α-Klotho decline in cartilage that triggers cartilage degeneration. FIG. 21A) Aging induces progressive α-Klotho decline in murine medial tibial plateaus. White arrows indicate α-Klotho-positive chondrocyte. White dash lines indicate cartilage surface. AC: articular cartilage. FIG. 21B) Cartilage in older adults (≥65 years) displays lower α-Klotho expression than that in young adults (<40 years). FIG. 21C) α-Klotho expression decreases with increased cartilage degeneration assessed by OARSI score in a sex-dependent manner. Statistical analysis was performed using linear regression analysis (FIGS. 21A and 21B), bootstrap linear regression analysis (FIG. 21C). P(x,y)<0.05 in FIG. 21C indicates that statistically significant relationship between both aging and α-Klotho decline (x-axis) and aging and OARSI score (y-axis). Data are presented as means±95% confidence intervals.

FIGS. 22A-22G. Direct effects of α-Klotho on cartilage degeneration and signaling pathway. FIG. 22A) Loss of function in α-Klotho (Klotho+/−) triggers cartilage degeneration in murine medial tibial plateaus in a sex-dependent manner. Black arrows indicate cartilage surface disruption. AC: articular cartilage. FIG. 22E) Cartilage in Klotho^{+/-} male mice displays higher surface roughness. FIG. 22C) Schematic showing the experimental protocol of 2-week α-Klotho infusion via an osmotic pump. FIG. 22D) α-Klotho infusion via an osmotic pump ameliorates age-related cartilage degeneration. Representative histological sections stained with Safranin-O/Fast Green are provided. Black arrows indicate cartilage surface disruption. AC: articular cartilage. OARSI score (0-24 points; higher value indicates more severe cartilage degeneration) and surface roughness are provided. Black dash lines indicate mean values of OARSI score and surface roughness in male young mice. FIG. 22E) Schematic showing the experimental flow of microarray from primary chondrocytes in patients with KOA as described in Chuchana et al. (Aging (Albany N.Y.), 10:1442-1453 (2018)). FIG. 22F) Gene set enrichment analysis plots for PI3K/Akt gene set showing the significantly changes after treatment of siRNA Klotho and recombinant human α-Klotho protein. FDR: false discovery ratio. NES: normalized enrichment score. FIG. 22G) Key elements of Insulin/PI3K/Akt pathway showing that α-Klotho inhibits Insulin/PI3K/Akt signaling in human KOA chondrocytes. Statistical analysis was performed using two-way ANOVA (FIGS. 22A and 22B) and Student t-test (FIG. 22D). Data are presented as means±95% confidence intervals.

FIGS. 23A-23I. Age-related α-Klotho decline accompanied with nuclear morphological alterations. FIG. 23A) Age-related change in proteins associated with nucleus envelop component in male mice. FIG. 23B) Aging increases Lamin A/C and B2 protein expression. FIG. 23C) Analytical flow of the nuclear morphological analysis (53 variables) using the Cell profiler. FIG. 23D) Representative nuclear images in murine medial tibial plateaus generated by Cell profiler. White dash lines indicate cartilage surface. FIG. 23E) Principal component analysis (PCA) showing the separate clusters in young and aged nuclear morphology. FIG. 23F) Heat map of the top 10 nuclear morphological variables contributing principal component 1, showing the age-related nuclear morphological alterations. Color indicates z-score in each variable. FIG. 23G) Murine and human cartilage samples share four nuclear features, in which higher age increases nuclear eccentricity (i.e., less roundness) in murine and human cartilage. FIG. 23H) Age-related increased nuclear eccentricity is associated with decreased α-Klotho expression. Sensitivity analysis excluding one outlier data showed a similar trend. FIG. 23) Schematic showing the relationship among chronological age, α-Klotho expression, and nuclear morphology. Linear regression model is provided showing the substantial and indendent contribution of nuclear morphology (Nuclearhuman) in the prediction of α-Klotho expression level in human cartilage beyond the chronological age effect (Agehuman). Coefficient of determinations are provided for mice (R2m) and human cartilage (R2h). Statistical analysis was performed using linear regression analysis (FIGS. 23G, 23H, and 23I). Data are presented as means±95% confidence intervals.

FIGS. 24A-24C. Matrix stiffness regulates chondrocyte α-Klotho expression. FIG. 24A) Schematic showing the experimental protocol. Primary chondrocytes isolated from young and aged cartilage were seeded onto polyacrylamide gels engineered to mimic a physiological range of knee cartilage ECM stiffnesses (5 kPa, 21 kPa, and 100 kPa). These range of stiffness differentially influence chondrocytes roundness (see FIG. 40). FIGS. 24B and 24C) Stiff substrate increases expression of Lamin A/C (FIG. 24B) and reduces α-Klotho and type II collagen (FIG. 24C) in chondrocytes regardless of age of the cell donor. Insets in the fluorescence microscope images highlight increased fluorescence signal in entire nucleus of chondrocytes cultured on stiff substrate. Data are presented as means±95% confidence intervals.

FIGS. 25A-J. BAPN injection improves α-Klotho expression and cartilage integrity in aged mice. FIG. 25A) Principal component analysis (PCA) showing the separate clusters in ECM proteins from young and aged cartilage. FIG. 25B) Principal component 2 in ECM protein expression has a link with Lamin A/C expression. FIG. 25C) Increased LOX expression is associated with increased Lamin A/C expression, indicating the connection between LOX-mediated increased collagen crosslinking and nuclear stiffness. FIG. 25D) Schematic showing the experimental protocol of BAPN daily injection. FIG. 25E) Representative Cell profiler-generated nuclear images in murine medial tibial plateaus after 4-week injection of saline or BAPN. White dash lines indicate cartilage surface. FIG. 25F) PCA showing the same cluster of (1) young and aged+BAPN injection and (2) aged and aged+saline injection. FIG. 25G) BAPN injection in aged mice decreases nuclear eccentricity towards young level. FIG. 25H) BAPN injection in aged mice improves α-Klotho expression. FIG. 25I) BAPN injection in aged mice improves cartilage integrity. Representative histological sections stained with Safranin-O/Fast Green are provided. Black arrow indicate loss of cartilage matrix. OARSI score (0-24 points; higher value indicates more severe cartilage degeneration) assessed by blinded assessor is provided. FIG. 25J) BAPN injection-induced decreased nuclear eccentricity (i.e., more roundness) is associated with higher α-Klotho expression. Statistical analysis was performed using linear regression analysis (FIGS. 25B, 25C, and 25J), Student t-test (FIGS. 25G-25I). Data are presented as means±95% confidence intervals.

FIG. 26. Female Aged vs. Young Mass Spectrometry Individual Protein Volcano Plot. Individually identified proteins were determined to be significantly different if the log 2(ratio of female aged vs young) was greater than 1 or less than −1 and the −log 10(p-value) was greater than 1.25.

FIG. 27. Female Middle-Aged vs. Young Mass Spectrometry Individual Protein Volcano Plot Individually identified proteins were determined to be significantly different if the log 2(ratio of female middle-aged vs young) was greater than 1 or less than −1 and the −log 10(p-value) was greater than 1.25.

FIG. 28. Individually identified proteins were determined to be significantly different if the log 2(ratio of male aged vs. female aged) was greater than 1 or less than −1 and the −log 10(p-value) was greater than 1.25.

FIG. 29. Individually identified proteins were determined to be significantly different if the log 2(ratio of male middle-aged vs. female middle-aged) was greater than 1 or less than −1 and the −log 10(p-value) was greater than 1.25.

FIG. 30. Individually identified proteins were determined to be significantly different if the log 2(ratio of male young vs. female young) was greater than 1 or less than −1 and the −log 10(p-value) was greater than 1.25.

FIG. 31. Male Aged vs. Young Mass Spectrometry Individual Protein Volcano Plot. Individually identified proteins were determined to be significantly different if the log 2(ratio of male aged vs young) was greater than 1 or less than −1 and the −log 10(p-value) was greater than 1.25.

FIG. 32. Male Middle-Aged vs. Young Mass Spectrometry Individual Protein Volcano Plot. Individually identified proteins were determined to be significantly different if the log 2(ratio of male middle-aged vs young) was greater than 1 or less than −1 and the −log 10(p-value) was greater than 1.25.

FIGS. 33A-33B. Female Aged vs. Young ROnToTool Pathway Analysis Statistics. Individual pathway statistics resulting from Female Aged vs Young Comparison. FIG. 33A) Complement and Coagulation Cascade. FIG. 33B) Focal Adhesion.

FIGS. 34A-34B. Female Middle-Aged vs. Young ROnToTool Pathway Analysis Statistics. Individual pathway statistics resulting from Female Aged vs Young Comparison. FIG. 34A) Whole Pathway Analysis Results. FIG. 34B) Complement and Coagulation Cascade.

FIGS. 35A-35D. Male Aged vs. Female Aged ROnToTool Pathway Analysis Statistics Individual pathway statistics resulting from Female Aged vs Young Comparison. FIG. 35A) Whole Pathway Analysis Results. FIG. 35B) Complement and Coagulation Cascade. FIG. 35C) SLE. FIG. 35D) COVID-19.

FIGS. 36A-36D. Male Middle-Aged vs. Female Middle-Aged ROnToTool Pathway Analysis Statistics Individual pathway statistics resulting from Female Aged vs Young Comparison. FIG. 36A) Whole Pathway Analysis Results. FIG. 36B) PPAR Signaling. FIG. 36C) Necroptosis. FIG. 36D) COVID-19.

FIGS. 37A-37E. Male Young vs. Female Young ROnToTool Pathway Analysis Statistics. Individual pathway statistics resulting from Female Aged vs Young Comparison. FIG. 37A) Whole Pathway Analysis Results. FIG. 37B) Coagulation and Complement Cascade. FIG. 37C) COVID-19. FIG. 37D) ECM Receptor Interaction. FIG. 37E) Staphylococcus aureus infection.

FIGS. 38A-38H. Male Aged vs. Young ROnToTool Pathway Analysis Statistics. Individual pathway statistics resulting from Male Aged vs Young Comparison. FIG. 38A) ECM Receptor Interaction, FIG. 38B) Focal Adhesion. FIG. 38C) TGF-Beta Signaling. FIG. 38D) PI3K/Akt Signaling. FIG. 38E) HPV Infection. FIG. 38F) Proteoglycans in Cancer. FIG. 38G) Cellular Senescence. FIG. 38H) Hippo Signaling.

FIGS. 39A-39B. Male Middle-Aged vs. Young ROnToTool Pathway Analysis Statistics. Individual pathway statistics resulting from Male Middle-Aged vs Young Comparison. FIG. 39A) Whole Pathways Results. FIG. 39B) PI3K/Akt Signaling.

FIGS. 40A-40B. Chondrocytes morphological changes in different stiffness of polyacrylamide gels. Primary chondrocytes cultured on physiological range of knee cartilage ECM stiffnesses (5 kPa, 21 kPa, and 100 kPa) displayed different morphology. Representative F-actin image in each stiffness (FIG. 40A). Principal component analysis (PCA) of 53 cell morphological variables showed the separate clusters between soft and stiff substrate. Form factor (i.e., roundness) is one of the cell morphological features sensitive to the different stiffness of substrate in both young and aged chondrocytes (FIG. 40B).

### DETAILED DESCRIPTION

This document provides methods and materials for treating osteoarthritis. For example, a mammal having, or at risk for developing, osteoarthritis can be treated by increasing the level of one or more Klotho polypeptides (e.g., one or more α-Klotho polypeptides) within cells (e.g., chondrocytes) within the mammal. A level of one or more Klotho polypeptides within cells within the mammal can be increased using any appropriate technique. In some cases, one or more Klotho polypeptides (e.g., one or more α-Klotho polypeptides) can be administered to a mammal having, or at risk for developing, osteoarthritis (e.g., knee osteoarthritis) to treat the mammal. In some cases, nucleic acid encoding one or more Klotho polypeptides (e.g., one or more α-Klotho polypeptides) can be administered to a mammal having, or at risk for developing, osteoarthritis (e.g., knee osteoarthritis) to treat the mammal.

In some cases, a particle (e.g., an extracellular vesicle such as an exosome) containing (a) one or more Klotho polypeptides (e.g., one or more α-Klotho polypeptides) and/or (b) nucleic acid (e.g., mRNA) encoding one or more Klotho polypeptides (e.g., one or more α-Klotho polypeptides) can be administered to a mammal having, or at risk for developing, osteoarthritis (e.g., knee osteoarthritis) to treat the mammal. In some cases, gene editing can be used to reduce or eliminate methylation of a promoter that directs expression of a Klotho polypeptide (e.g., an α-Klotho polypeptide), thereby increasing expression of a Klotho polypeptide (e.g., an α-Klotho polypeptide) within a mammal having, or at risk for developing, osteoarthritis (e.g., knee osteoarthritis).

The term “increased level” as used herein with respect to a level of a Klotho polypeptide (e.g., an α-Klotho polypeptide) in a mammal having, or at risk for developing, osteoarthritis (e.g., knee osteoarthritis) refers to any level that is greater than the level of that Klotho polypeptide observed in that mammal prior to being treated as described herein. In some cases, an increased level of a Klotho polypeptide can be a level that is at least 5 percent (e.g., at least 10, at least 15, at least 20, at least 25, at least 35, at least 50, at least 75, at least 100, or at least 150 percent) higher than the level of that Klotho polypeptide prior to being treated as described herein. In some cases, when samples have an undetectable level of a Klotho polypeptide prior to treatment as described herein, an increased level can be any detectable level of a Klotho polypeptide. It will be appreciated that levels from comparable samples are used when determining whether or not a particular level is an increased level.

Any appropriate mammal having, or at risk for developing, osteoarthritis (e.g., knee osteoarthritis) can be treated as described herein (e.g., by increasing the level of one or more Klotho polypeptides in one or more cells within the mammal). Examples of mammals that can have osteoarthritis and can be treated as described herein include, without limitation, humans, non-human primates (e.g., monkeys), dogs, cats, horses, cows, pigs, sheep, mice, rats, rabbits, hamsters, and goats. In some cases, a human having osteoarthritis can be treated as described herein.

When treating a mammal (e.g., a human) having, or at risk for developing, osteoarthritis as described herein (e.g., by increasing the level of one or more Klotho polypeptides in one or more cells within the mammal), the osteoarthritis can be any type of osteoarthritis. For example, osteoarthritis can affect any part of a mammal (e.g., any part of a mammal's body). Examples of parts of a mammal that can be affected by osteoarthritis include, without limitation, knees, hands, hips, spine, feet, shoulders, and elbows. In some cases, a mammal having knee osteoarthritis can be treated as described herein.

In some cases, the methods described herein can include identifying a mammal (e.g., a human) as having, or at risk for developing, osteoarthritis (e.g., knee osteoarthritis). Any appropriate method can be used to identify a mammal as having osteoarthritis. For example, physical examinations (e.g., for joint tenderness, swelling, redness, and flexibility), imaging tests (e.g., X-rays and magnetic resonance imaging (MRI)), and/or laboratory tests (e.g., blood tests and joint fluid analysis) can be used to identify mammals as having osteoarthritis. In some cases, a presence of a reduced level of one or more Klotho polypeptides (e.g., one or more α-Klotho polypeptides) in a sample (e.g., biopsy sample) obtained from a mammal can be used to identify the mammal as being at risk for developing osteoarthritis. A sample can be any appropriate sample. In some cases, a sample can be a fluid sample (e.g., a blood sample). In some cases, a sample can be tissue sample (e.g., a biopsy). Examples of samples that can be obtained from a mammal and assessed for a reduced level of one or more Klotho polypeptides include, without limitation, blood samples (e.g., whole blood, serum, and plasma), cartilage samples, and joint fluid samples.

Once identified as having, or as being at risk for developing, osteoarthritis (e.g., knee osteoarthritis), a mammal (e.g., a human) can be administered, or instructed to self-administer, one or more Klotho polypeptides (e.g., one or more α-Klotho polypeptides), nucleic acid encoding one or more Klotho polypeptides (e.g., one or more α-Klotho polypeptides), and/or one or more gene editing components designed to reduce or eliminate methylation of a promoter that directs expression of one or more Klotho polypeptides (e.g., one or more α-Klotho polypeptides) to increase the level of one or more Klotho polypeptides in one or more cells within that mammal.

In some cases, one or more Klotho polypeptides (e.g., one or more α-Klotho polypeptides) can be administered to a mammal (e.g., a human) having, or at risk for developing, osteoarthritis (e.g., knee osteoarthritis) to treat the mammal. For example, a mammal having, or at risk for developing, osteoarthritis can be administered or can self-administer a composition containing one or more Klotho polypeptides (e.g., a composition containing one or more α-Klotho polypeptides). In some cases, a composition containing one or more Klotho polypeptides can be administered to a mammal having osteoarthritis to increase the level of one or more Klotho polypeptides within that mammal.

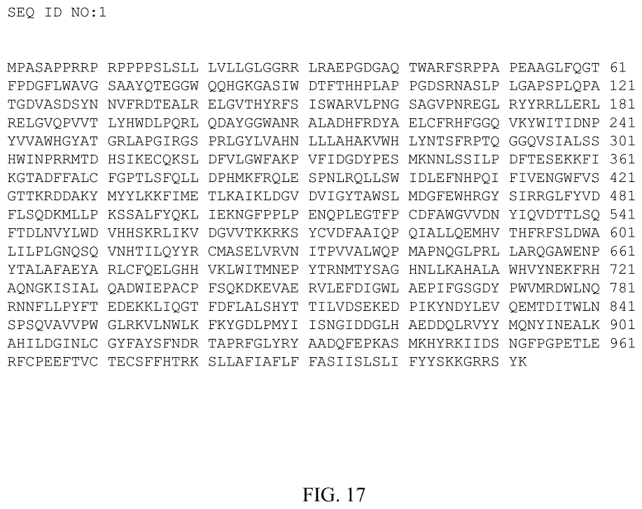

A Klotho polypeptide can be any appropriate Klotho polypeptide. In some cases, a Klotho polypeptide can be a membrane-bound Klotho. In some cases, a Klotho polypeptide can be a circulating Klotho polypeptide (e.g., a Klotho polypeptide present in the bloodstream of a mammal). In some cases, a Klotho polypeptide can be from about 5 kDa to about 140 kDa (e.g., from about 5 kDa to about 135 kDa, from about 15 kDa to about 140 kDa, from about 50 kDa to about 140 kDa, or from about 120 kDa to about 135 kDa). Examples of Klotho polypeptides that can be used as described herein (e.g., to treat a mammal having, or at risk for developing, osteoarthritis) include, without limitation, α-Klotho, β-Klotho, and γ-Klotho polypeptides. For example, a mammal having, or at risk for developing, osteoarthritis (e.g., knee osteoarthritis) can be administered or can self-administer one or more α-Klotho polypeptides. An example of a Klotho polypeptide that can be used as described herein includes, without limitation, a human Klotho polypeptide having the amino acid sequence set forth in National Center for Biotechnology Information (NCBI) GenBank® Accession No. NP_004786.2, XP_006719958.1, or BAA23382.1. A representative human Klotho polypeptide sequence is set forth in FIG. 17 as SEQ ID NO:1.

In some cases, nucleic acid encoding one or more Klotho polypeptides (e.g., one or more α-Klotho polypeptides) can be administered to a mammal (e.g., a human) having, or at risk for developing, osteoarthritis (e.g., knee osteoarthritis) to treat the mammal. For example, a mammal having, or at risk for developing, osteoarthritis can be administered or can self-administer a composition containing nucleic acid encoding one or more Klotho polypeptides (e.g., a composition containing nucleic acid encoding one or more α-Klotho polypeptides). In some cases, a composition containing nucleic acid encoding one or more Klotho polypeptides can be administered to a mammal having osteoarthritis to increase the level of one or more osteoarthritis polypeptides within that mammal.

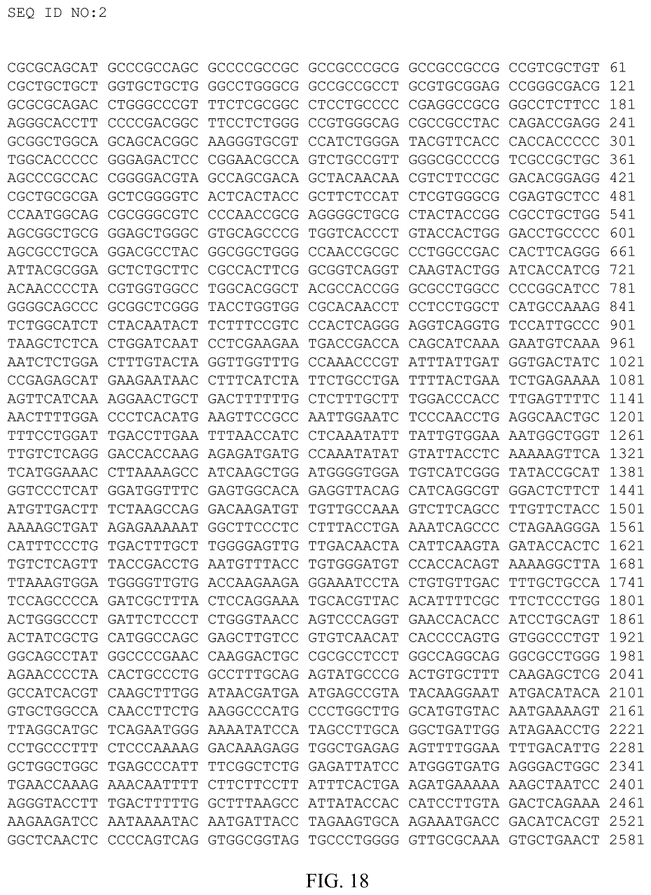

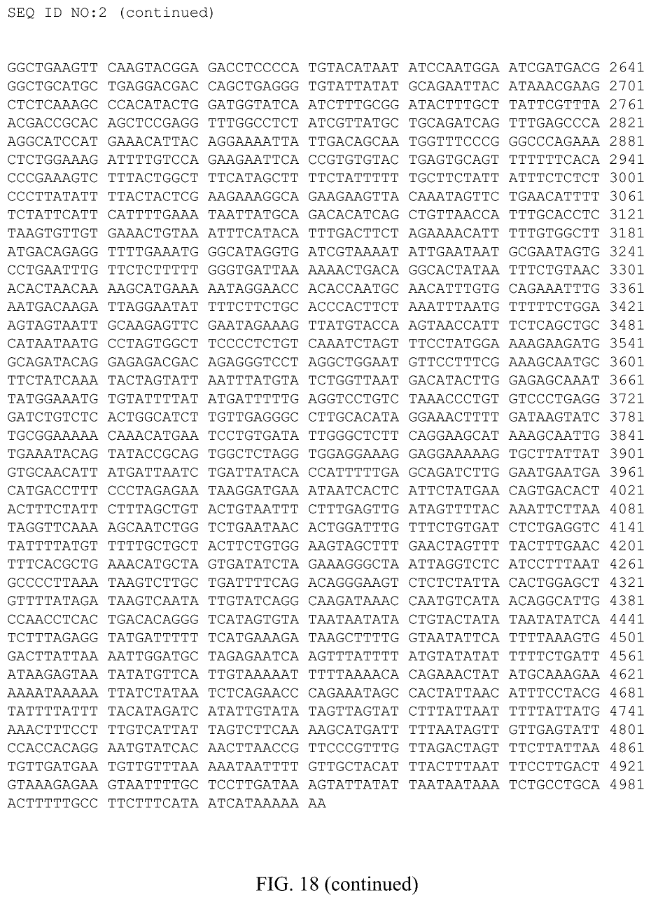

A nucleic acid encoding a Klotho polypeptide (e.g., an α-Klotho polypeptide) can be any appropriate nucleic acid. A nucleic acid encoding a Klotho polypeptide can encode any Klotho polypeptide described herein. In some cases, a nucleic acid encoding a Klotho polypeptide can encode an α-Klotho polypeptide. An example of a nucleic acid encoding a Klotho polypeptide that can be used as described herein includes, without limitation, nucleic acid encoding a human Klotho sequence as set forth in GenBank® Accession No. NM_004795.4 or AB005142.1. A representative nucleic acid sequence encoding a human Klotho polypeptide is set forth in FIG. 18 as SEQ ID NO:2.

In some cases, nucleic acid encoding a Klotho polypeptide (e.g., an α-Klotho polypeptide) can be in a nucleic acid vector (e.g., an expression vector). In some cases, a vector can be a plasmid. In some cases, a vector can be viral vector. Examples of viral vectors that can be used to deliver nucleic acid encoding one or more Klotho polypeptides (e.g., an α-Klotho polypeptide) to a mammal to treat osteoarthritis as described herein include, without limitation, adenoviral vectors, adeno-associated viral (AAV) vectors, lentiviral vectors, herpes viral vectors, retroviral vectors, and vaccinia viral vectors.

An expression vector (e.g., viral vector) can include one or more elements involved in expressing a polypeptide (e.g., a Klotho polypeptide) from a nucleic acid sequence within the vector (e.g., a ribosomal binding site and start codon, a termination codon, and/or a transcription termination sequence). In cases where a nucleic acid encoding a Klotho polypeptide is a vector, the vector also can include one or more regulatory elements (e.g., enhancers and promotes) that can enhance expression of a polypeptide (e.g., a Klotho polypeptide) from a nucleic acid sequence within the vector. A promoter can be a constitutive promoter or an inducible promoter. A promoter can be a ubiquitous promoter or a tissue/cell-specific promoter (e.g., a chondrocyte-specific promoter). Examples of promoters that can increase expression of a polypeptide (e.g., a Klotho polypeptide) from a nucleic acid sequence within a vector include, without limitation, chondrocyte-specific promoters such as a collagen type II promoter, a collagen type XI promoter, and an aggrecan promotor. In cases where a nucleic acid encoding a Klotho polypeptide is a vector, the vector also can include an origin of replication, a selectable marker, and/or a nucleic acid encoding a detectable label.

In some cases, one or more particles (e.g., one or more extracellular vesicles such as exosomes) containing (a) one or more Klotho polypeptides (e.g., one or more α-Klotho polypeptides) and/or (b) nucleic acid encoding one or more one or more Klotho polypeptides (e.g., one or more α-Klotho polypeptides) can be administered to a mammal having, or at risk for developing, osteoarthritis (e.g., knee osteoarthritis) to treat the mammal. For example, a mammal having, or at risk for developing, osteoarthritis can be administered or can self-administer a composition containing one or more particles containing (a) one or more Klotho polypeptides and/or (b) nucleic acid encoding one or more one or more Klotho polypeptides (e.g., a composition containing an α-Klotho polypeptide and/or nucleic acid encoding an α-Klotho polypeptide). In some cases, a composition containing nucleic acid encoding one or more Klotho polypeptides can be administered to a mammal having osteoarthritis to increase the level of one or more Klotho polypeptides within that mammal.

In some cases, gene editing can be used to reduce or eliminate methylation of a promoter that directs expression of one or more Klotho polypeptides (e.g., one or more α-Klotho polypeptides) within a mammal having, or at risk for developing, osteoarthritis (e.g., knee osteoarthritis) to treat the mammal. For example, a mammal having, or at risk for developing, osteoarthritis can be administered or can self-administer a composition containing one or more gene editing components designed to replace a methylation site of a promoter that directs expression of one or more Klotho polypeptides with a promoter sequence that lacks that methylation site to increase the level of one or more Klotho polypeptides (e.g., an α-Klotho polypeptide) in one or more cells within that mammal (e.g., human).

Gene editing components designed to remove or replace one or more methylation sites of a promoter that directs expression of one or more Klotho polypeptides can include any appropriate gene editing components. Examples of gene editing components that can be designed to reduce or eliminate methylation of a promoter that directs expression of one or more Klotho polypeptides include, without limitation, zinc finger nucleases (ZFNs), TALE nucleases (TALENs), and clustered regularly interspaced palindromic repeats (CRISPR)/Cas9 systems. When a CRISPR/Cas9 system is used to reduce or eliminate methylation of a promoter that directs expression of one or more Klotho polypeptides, the Cas9 component of a CRISPR/Cas9 system can be any appropriate Cas9 (e.g., a Staphylococcus aureus Cas9 (saCas9)). The nucleic acid and/or polypeptide sequences of such genome editing molecules can be as described elsewhere (see, e.g., Mani et al., Biochemical and Biophysical Research Communications, 335:447-457 (2005); Campbell et al., Circulation Research, 113:571-587 (2013); Cong et al., Science, 339:819-823 (2013); and Ran et al., Nature, 520:186-191 (2015)).

Gene editing components designed to remove or replace one or more methylation sites of a promoter that directs expression of one or more Klotho polypeptides can be designed to target a promoter that directs expression of any appropriate Klotho polypeptide described herein. In some cases, gene editing components designed to remove or replace one or more methylation sites of a promoter that directs expression of one or more Klotho polypeptides can target a promoter that directs expression of an α-Klotho polypeptide such as a Klotho promoter.

Any appropriate method can be used to deliver one or more gene editing components (e.g., a CRISPR/Cas9 system) described herein or nucleic acid encoding one or more gene editing components described herein to a cell (e.g., a cell within a mammal). For example, a vector (e.g., a viral vector) can be used to deliver nucleic acid encoding a CRISPR/Cas9 system described herein to cells within a mammal (e.g. a human). In some cases, a single vector can be designed to deliver both a nucleic acid encoding the Cas9 component (e.g., an saCas9) and the targeting guide RNA of a CRISPR/Cas9 system.

In some cases, treating a mammal (e.g., a human) having, or at risk for developing, osteoarthritis (e.g., knee osteoarthritis) as described herein (e.g., by increasing the level of one or more Klotho polypeptides in one or more cells within the mammal) can be effective to reduce or eliminate one or more symptoms of osteoarthritis. Examples of symptoms of osteoarthritis that can be reduced as described herein include, without limitation, joint pain, joint stiffness, joint tenderness, loss of joint flexibility, grating sensation in a joint, popping or crackling of a joint, bone spurs, and joint swelling. For example, (a) one or more Klotho polypeptides, (b) nucleic acid encoding one or more Klotho polypeptides, and/or (c) gene editing components designed to reduce or eliminate methylation of a promoter that directs expression of one or more Klotho polypeptides can be administered to a mammal in need thereof (e.g., a human having, or at risk for developing, osteoarthritis) as described herein to reduce one or more symptoms of osteoarthritis within the mammal by, for example, 10, 20, 30, 40, 50, 60, 70, 80, 90, 95, or more percent.

In some cases, treating a mammal (e.g., a human) having, or at risk for developing, osteoarthritis (e.g., knee osteoarthritis) as described herein (e.g., by increasing the level of one or more Klotho polypeptides in one or more cells within the mammal) can be effective to improve cartilage integrity within the mammal. For example, (a) one or more Klotho polypeptides, (b) nucleic acid encoding one or more Klotho polypeptides, and/or (c) one or more gene editing components designed to reduce or eliminate methylation of a promoter that directs expression of one or more Klotho polypeptides can be administered to a mammal in need thereof (e.g., a human having, or at risk for developing, osteoarthritis) as described herein to improve cartilage integrity within the mammal by, for example, 10, 20, 30, 40, 50, 60, 70, 80, 90, 95, or more percent.

In some cases, treating a mammal (e.g., a human) having, or at risk for developing, osteoarthritis (e.g., knee osteoarthritis) as described herein (e.g., by increasing the level of one or more Klotho polypeptides in one or more cells within the mammal) can be effective to reduce or eliminate cartilage degeneration within the mammal. For example, (a) one or more Klotho polypeptides, (b) nucleic acid encoding one or more Klotho polypeptides, and/or (c) one or more gene editing components designed to reduce or eliminate methylation of a promoter that directs expression of one or more Klotho polypeptides can be administered to a mammal in need thereof (e.g., a human having, or at risk for developing, osteoarthritis) as described herein to reduce cartilage degeneration within the mammal by, for example, 10, 20, 30, 40, 50, 60, 70, 80, 90, 95, or more percent.

In some cases, treating a mammal (e.g., a human) having, or at risk for developing, osteoarthritis (e.g., knee osteoarthritis) as described herein (e.g., by increasing the level of one or more Klotho polypeptides in one or more cells within the mammal) can be effective to reduce or eliminate surface irregularity of cartilage within the mammal. For example, (a) one or more Klotho polypeptides, (b) nucleic acid encoding one or more Klotho polypeptides, and/or (c) one or more gene editing components designed to reduce or eliminate methylation of a promoter that directs expression of one or more Klotho polypeptides can be administered to a mammal in need thereof (e.g., a human having, or at risk for developing, osteoarthritis) as described herein to reduce surface irregularity of cartilage within the mammal by, for example, 10, 20, 30, 40, 50, 60, 70, 80, 90, 95, or more percent.

In some cases, treating a mammal (e.g., a human) having, or at risk for developing, osteoarthritis (e.g., knee osteoarthritis) as described herein (e.g., by increasing the level of one or more Klotho polypeptides in one or more cells within the mammal) can be effective to improve an OARSI score of cartilage within the mammal. An OARSI score can be determined as described elsewhere (see, e.g., Pritzker, Osteoarthritis Cartilage, 14(1):13-29 (2006)). For example, (a) one or more Klotho polypeptides, (b) nucleic acid encoding one or more Klotho polypeptides, and/or (c) one or more gene editing components designed to reduce or eliminate methylation of a promoter that directs expression of one or more Klotho polypeptides can be administered to a mammal in need thereof (e.g., a human having, or at risk for developing, osteoarthritis) as described herein to achieve an OARSI score of from 0 to 24.

In some cases, treating a mammal (e.g., a human) having, or at risk for developing, osteoarthritis (e.g., knee osteoarthritis) as described herein (e.g., by increasing the level of one or more Klotho polypeptides in one or more cells within the mammal) can be effective to improve a modified MANKIN score of cartilage within the mammal. A modified MANKIN score can be determined as described elsewhere (see, e.g., Henson, BMC Musculoskelet. Disord., 9:94 (2008)). For example, (a) one or more Klotho polypeptides, (b) nucleic acid encoding one or more Klotho polypeptides, and/or (c) one or more gene editing components designed to reduce or eliminate methylation of a promoter that directs expression of one or more Klotho polypeptides can be administered to a mammal in need thereof (e.g., a human having, or at risk for developing, osteoarthritis) as described herein to achieve a total modified MANKIN score of from 0 to 15.

In some cases, treating a mammal (e.g., a human) having, or at risk for developing, osteoarthritis (e.g., knee osteoarthritis) as described herein (e.g., by increasing the level of one or more Klotho polypeptides in one or more cells within the mammal) can be effective to improve nuclear morphology in one or more chondrocytes within the mammal. For example, (a) one or more Klotho polypeptides, (b) nucleic acid encoding one or more Klotho polypeptides, and/or (c) one or more gene editing components designed to reduce or eliminate methylation of a promoter that directs expression of one or more Klotho polypeptides can be administered to a mammal in need thereof (e.g., a human having, or at risk for developing, osteoarthritis) as described herein to improve a nuclear aspect ratio, nuclear circularity, and/or nuclear size of one or more chondrocytes within the mammal.

This document also provides compositions containing (a) one or more Klotho polypeptides (e.g., an α-Klotho polypeptide), (b) nucleic acid encoding one or more Klotho polypeptides (e.g., a nucleic acid vector encoding an α-Klotho polypeptide), (c) particles containing (i) one or more Klotho polypeptides and/or (ii) nucleic acid encoding one or more Klotho polypeptides, and/or (d) one or more gene editing components designed to reduce or eliminate methylation of a promoter that directs expression of a Klotho polypeptide (e.g., an a-Klotho polypeptide). For example, one or more Klotho polypeptides, nucleic acid encoding one or more Klotho polypeptides, particles containing one or more Klotho polypeptides and/or nucleic acid encoding one or more Klotho polypeptides, and/or one or more gene editing components designed to reduce or eliminate methylation of a promoter that directs expression of a Klotho polypeptide can be formulated into a composition (e.g., a pharmaceutically acceptable composition) for administration to a mammal (e.g., a human) having, or at risk for developing, osteoarthritis (e.g., knee osteoarthritis). For example, one or more Klotho polypeptides, nucleic acid encoding one or more Klotho polypeptides, particles containing one or more Klotho polypeptides and/or nucleic acid encoding one or more Klotho polypeptides, and/or one or more gene editing components designed to reduce or eliminate methylation of a promoter that directs expression of a Klotho polypeptide can be formulated together with one or more pharmaceutically acceptable carriers (additives) and/or diluents. Pharmaceutically acceptable carriers, fillers, and vehicles that may be used in a pharmaceutical composition described herein include, without limitation, saline, dimethyl sulfoxide (DMSO), ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, and wool fat.

A composition including one or more Klotho polypeptides (e.g., an α-Klotho polypeptide), nucleic acid encoding one or more Klotho polypeptides (e.g., a nucleic acid vector encoding an α-Klotho polypeptide), particles containing one or more Klotho polypeptides (e.g., an α-Klotho polypeptide) and/or nucleic acid encoding one or more Klotho polypeptides (e.g., an α-Klotho polypeptide), and/or one or more gene editing components designed to reduce or eliminate methylation of a promoter that directs expression of a Klotho polypeptide (e.g., an α-Klotho polypeptide) can be designed for oral or parenteral (including intra-articular, intraperitoneal, subcutaneous, intramuscular, intravenous, and intradermal) administration to a mammal (e.g., a human) having, or at risk for developing, osteoarthritis (e.g., knee osteoarthritis). Compositions suitable for oral administration include, without limitation, liquids, tablets, capsules, pills, powders, gels, and granules. Compositions suitable for parenteral administration include, without limitation, aqueous and non-aqueous sterile injection solutions that can contain anti-oxidants, buffers, bacteriostats, and solutes that render the formulation isotonic with the blood of the intended recipient. In some cases, a composition including one or more Klotho polypeptides and/or nucleic acid encoding one or more Klotho polypeptides can be formulated for parenteral administration (e.g., intra-articular injection and intravenous injection).

A composition including one or more Klotho polypeptides (e.g., an α-Klotho polypeptide), nucleic acid encoding one or more Klotho polypeptides (e.g., a nucleic acid vector encoding an α-Klotho polypeptide), particles containing one or more Klotho polypeptides (e.g., an α-Klotho polypeptide) and/or nucleic acid encoding one or more Klotho polypeptides (e.g., an α-Klotho polypeptide), and/or one or more gene editing components designed to reduce or eliminate methylation of a promoter that directs expression of a Klotho polypeptide (e.g., an α-Klotho polypeptide) can be designed for any type of release (e.g., release of the one or more Klotho polypeptides, the nucleic acid encoding one or more Klotho polypeptides, and/or the one or more gene editing components from the composition) into the mammal that the composition is administered to (e.g., a mammal having, or at risk for developing, osteoarthritis). For example, a composition including one or more Klotho polypeptides, nucleic acid encoding one or more Klotho polypeptides, particles containing one or more Klotho polypeptides and/or nucleic acid encoding one or more Klotho polypeptides, and/or one or more gene editing components designed to reduce or eliminate methylation of a promoter that directs expression of a Klotho polypeptide can be designed for immediate release, slow release, or extended release of the Klotho polypeptide, nucleic acid, or gene editing component(s).

A composition including one or more Klotho polypeptides (e.g., an α-Klotho polypeptide), nucleic acid encoding one or more Klotho polypeptides (e.g., a nucleic acid vector encoding an α-Klotho polypeptide), particles containing one or more Klotho polypeptides (e.g., an α-Klotho polypeptide) and/or nucleic acid encoding one or more Klotho polypeptides (e.g., an α-Klotho polypeptide), and/or one or more gene editing components designed to reduce or eliminate methylation of a promoter that directs expression of a Klotho polypeptide (e.g., an α-Klotho polypeptide) can be administered locally or systemically to a mammal (e.g., a human) having, or at risk for developing, osteoarthritis (e.g., knee osteoarthritis). For example, a composition including one or more Klotho polypeptides, nucleic acid encoding one or more Klotho polypeptides, particles containing one or more Klotho polypeptides and/or nucleic acid encoding one or more Klotho polypeptides, and/or one or more gene editing components designed to reduce or eliminate methylation of a promoter that directs expression of a Klotho polypeptide can be administered locally by intra-articular administration to a mammal having, or at risk for developing, osteoarthritis. For example, a composition including one or more Klotho polypeptides, nucleic acid encoding one or more Klotho polypeptides, particles containing one or more Klotho polypeptides and/or nucleic acid encoding one or more Klotho polypeptides, and/or one or more gene editing components designed to reduce or eliminate methylation of a promoter that directs expression of a Klotho polypeptide can be administered systemically by intravenous administration to a mammal having, or at risk for developing, osteoarthritis.

A composition including one or more Klotho polypeptides (e.g., an α-Klotho polypeptide), nucleic acid encoding one or more Klotho polypeptides (e.g., a nucleic acid vector encoding an α-Klotho polypeptide), particles containing one or more Klotho polypeptides (e.g., an α-Klotho polypeptide) and/or nucleic acid encoding one or more Klotho polypeptides (e.g., an α-Klotho polypeptide), and/or one or more gene editing components designed to reduce or eliminate methylation of a promoter that directs expression of a Klotho polypeptide (e.g., an α-Klotho polypeptide) can be administered to a mammal (e.g., a human) having, or at risk for developing, osteoarthritis (e.g., knee osteoarthritis) in any appropriate dose(s). Effective doses can vary depending on the severity of the osteoarthritis, the risk for developing osteoarthritis, the route of administration, the age and general health condition of the subject, excipient usage, the possibility of co-usage with other therapeutic treatments such as use of other agents, and the judgment of the treating physician. In cases where a composition includes one or more Klotho polypeptides (e.g., an a-Klotho polypeptide), an effective dose of that composition can be from about 5 picograms of Klotho polypeptides per milliliter (pg/mL) liquid (e.g., saline) to about 6 μg/mL (e.g., from about 5 pg/mL to about 5 μg/mL, from about 5 pg/mL to about 5 μg/mL, from about 5 pg/mL to about 1 μg/mL, from about 5 pg/mL to about 0.5 μg/mL, from about 5 pg/mL to about 0.1 μg/mL, from about 5 pg/mL to about 0.05 μg/mL, from about 50 pg/mL to about 1 μg/mL, from about 500 pg/mL to about 1 μg/mL, from about 1 ng/mL to about 1 μg/mL, or from about 100 ng/mL to about 500 ng/mL). In some cases, a composition including a Klotho polypeptide (e.g., α-Klotho polypeptide) can be from about 100 pg/mL to about 500 pg/mL (e.g., about 324 pg/mL). In some cases where a composition includes an α-Klotho polypeptide, the composition can be administered to deliver from about 0.001 μg to about 500 μg (e.g., from about 0.01 μg to about 500 from about 0.05 μg to about 500 μg, from about 0.1 μg to about 500 μg, from about 1 μg to about 500 μg, from about 10 μg to about 500 μg, from about 100 μg to about 500 μg, from about 0.001 μg to about 250 μg, from about 0.001 μg to about 100 μg, from about 0.001 μg to about 50 μg, from about 0.001 μg to about 5 μg, from about 0.1 μg to about 250 μg, from about 1 μg to about 100 μg, from about 5 μg to about 50 μg, from about 10 μg to about 50 μg, or from about 10 μg to about 30 μg) of an α-Klotho polypeptide per kg body weight of a mammal (e.g., a human). In cases where a composition includes one or more viral vectors (e.g., an AAV vector) used to deliver nucleic acid encoding one or more Klotho polypeptides (e.g., an α-Klotho polypeptide), an effective dose of that composition can be from about 1×10² vector genomes (vg) per dose to about 1×10⁹ vg per dose. In some cases, a composition including AAV vectors used to deliver nucleic acid encoding one or more Klotho polypeptides can be about 1×10⁸ vg per dose. An effective amount of a composition including one or more Klotho polypeptides (e.g., an α-Klotho polypeptide), nucleic acid encoding one or more Klotho polypeptides (e.g., a nucleic acid vector encoding an α-Klotho polypeptide), particles containing one or more Klotho polypeptides (e.g., an α-Klotho polypeptide) and/or nucleic acid encoding one or more Klotho polypeptides (e.g., an α-Klotho polypeptide), and/or one or more gene editing components designed to reduce or eliminate methylation of a promoter that directs expression of a Klotho polypeptide (e.g., an α-Klotho polypeptide) can be any amount that reduces the severity and/or one or more symptoms of a condition being treated (e.g., osteoarthritis such as knee osteoarthritis) without producing significant toxicity to the mammal. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, severity of the osteoarthritis, and risk for developing osteoarthritis may require an increase or decrease in the actual effective amount administered.

A composition including one or more Klotho polypeptides (e.g., an α-Klotho polypeptide), nucleic acid encoding one or more Klotho polypeptides (e.g., a nucleic acid vector encoding an α-Klotho polypeptide), particles containing one or more Klotho polypeptides (e.g., an α-Klotho polypeptide) and/or nucleic acid encoding one or more Klotho polypeptides (e.g., an α-Klotho polypeptide), and/or one or more gene editing components designed to reduce or eliminate methylation of a promoter that directs expression of a Klotho polypeptide (e.g., an α-Klotho polypeptide) can be administered to a mammal (e.g., a human) having, or at risk for developing, osteoarthritis (e.g., knee osteoarthritis) in any appropriate frequency. The frequency of administration can be any frequency that reduces the severity of the osteoarthritis and/or one or more symptoms of osteoarthritis without producing significant toxicity to the mammal. For example, the frequency of administration can be from about every three days to about ten times a day, from about every other day to about five times a day, or from about one time a day to about two times a day. In some cases, the frequency of administration can be once a day. The frequency of administration can remain constant or can be variable during the duration of treatment. As with the effective amount, various factors can influence the actual frequency of administration used for a particular application. For example, the effective amount, duration of treatment, use of multiple treatment agents, route of administration, severity of the osteoarthritis, and risk for developing osteoarthritis may require an increase or decrease in administration frequency.

A composition including one or more Klotho polypeptides (e.g., an α-Klotho polypeptide), nucleic acid encoding one or more Klotho polypeptides (e.g., a nucleic acid vector encoding an α-Klotho polypeptide), particles containing one or more Klotho polypeptides (e.g., an α-Klotho polypeptide) and/or nucleic acid encoding one or more Klotho polypeptides (e.g., an α-Klotho polypeptide), and/or one or more gene editing components designed to reduce or eliminate methylation of a promoter that directs expression of a Klotho polypeptide (e.g., an α-Klotho polypeptide) can be administered to a mammal (e.g., a human) having, or at risk for developing, osteoarthritis (e.g., knee osteoarthritis) for any appropriate duration. An effective duration for administering a composition including one or more Klotho polypeptides, nucleic acid encoding one or more Klotho polypeptides, particles containing one or more Klotho polypeptides and/or nucleic acid encoding one or more Klotho polypeptides, and/or one or more gene editing components designed to reduce or eliminate methylation of a promoter that directs expression of a Klotho polypeptide can be any duration that reduces the severity of the osteoarthritis and/or one or more symptoms of the osteoarthritis without producing significant toxicity to the mammal. For example, the effective duration can vary from several days to several months or years to a lifetime. In some cases, the effective duration for the treatment of osteoarthritis can range in duration from about 2 days to about a week. Multiple factors can influence the actual effective duration used for a particular treatment. For example, an effective duration can vary with the frequency of administration, effective amount, use of multiple treatment agents, route of administration, severity of the osteoarthritis, and risk for developing osteoarthritis.

In some cases, one or more Klotho polypeptides (e.g., an α-Klotho polypeptide), nucleic acid encoding one or more Klotho polypeptides (e.g., a nucleic acid vector encoding an α-Klotho polypeptide), particles containing one or more Klotho polypeptides (e.g., an α-Klotho polypeptide) and/or nucleic acid encoding one or more Klotho polypeptides (e.g., an a-Klotho polypeptide), and/or one or more gene editing components designed to reduce or eliminate methylation of a promoter that directs expression of a Klotho polypeptide (e.g., an a-Klotho polypeptide) can be administered to a mammal (e.g., a human) having, or at risk for developing, osteoarthritis (e.g., knee osteoarthritis) as the sole active ingredient. For example, one or more Klotho polypeptides, nucleic acid encoding one or more Klotho polypeptides, particles containing one or more Klotho polypeptides and/or nucleic acid encoding one or more Klotho polypeptides, and/or one or more gene editing components designed to reduce or eliminate methylation of a promoter that directs expression of a Klotho polypeptide can be administered to a mammal having, or at risk for developing, osteoarthritis as the sole active ingredient used to treat osteoarthritis. In some cases, one or more Klotho polypeptides or nucleic acid encoding one or more Klotho polypeptides can be administered as the sole active ingredient to a mammal in need thereof (e.g., a mammal such as a human having, or at risk for developing, osteoarthritis).

In some cases, one or more Klotho polypeptides (e.g., an α-Klotho polypeptide), nucleic acid encoding one or more Klotho polypeptides (e.g., a nucleic acid vector encoding an α-Klotho polypeptide), particles containing one or more Klotho polypeptides (e.g., an α-Klotho polypeptide) and/or nucleic acid encoding one or more Klotho polypeptides (e.g., an a-Klotho polypeptide), and/or one or more gene editing components designed to reduce or eliminate methylation of a promoter that directs expression of a Klotho polypeptide (e.g., an a-Klotho polypeptide) can be administered to a mammal (e.g., a human) having, or at risk for developing, osteoarthritis (e.g., knee osteoarthritis) with one or more additional agents and/or therapies. For example, one or more Klotho polypeptides and/or nucleic acid encoding one or more Klotho polypeptides can be administered to a mammal having, or at risk for developing, osteoarthritis with one or more additional agents or therapies used to treat osteoarthritis. Examples of additional agents or therapies that can be used to treat osteoarthritis include, without limitation, acetaminophen, nonsteroidal anti-inflammatory drugs (NSAIDs; e.g., ibuprofen and naproxen sodium), duloxetine, physical therapy, occupational therapy, cortisone injections, lubrication injections, and biomechanical interventions (e.g., knee braces or foot orthoses). In some cases, additional agents or therapies used to treat osteoarthritis can be as described elsewhere (see, e.g., McAlindon, Osteoarthritis Cartilage, 22(3):363-88 (2014)). In cases where one or more Klotho polypeptides, nucleic acid encoding one or more Klotho polypeptides, and/or one or more gene editing components designed to reduce or eliminate methylation of a promoter that directs expression of a Klotho polypeptide are used in combination with one or more additional agents or therapies, the one or more additional agents or therapies can be administered at the same time (e.g., in a single composition or in separate compositions) or independently. For example, a composition including one or more Klotho polypeptides, nucleic acid encoding one or more Klotho polypeptides, particles containing one or more Klotho polypeptides and/or nucleic acid encoding one or more Klotho polypeptides, and/or one or more gene editing components designed to reduce or eliminate methylation of a promoter that directs expression of a Klotho polypeptide can be administered first, and the one or more additional agents or therapies can be administered second, or vice versa.

In some cases, methods described herein can include monitoring a course of treatment and/or the severity of one or more symptoms related to the condition being treated (e.g., osteoarthritis such as knee osteoarthritis). Any appropriate method can be used to determine whether or not the severity of a symptom is reduced. For example, the severity of osteoarthritis can be assessed using any appropriate methods and/or techniques and can be assessed at different time points. For example, physical examinations (e.g., for joint tenderness, swelling, redness, and flexibility), imaging tests (e.g., X-rays and MRI), and/or laboratory tests (e.g., blood tests and joint fluid analysis) can be assessed to determine the severity of osteoarthritis.

In some cases, methods described herein can include monitoring a mammal being treated as described herein for toxicity. The level of toxicity, if any, can be determined by assessing a mammal's clinical signs and symptoms before and after administering a known amount of a particular composition. It is noted that the effective amount of a particular composition administered to a mammal can be adjusted according to a desired outcome as well as the mammal's response and level of toxicity.

The invention will be further described in the following examples, which do not limit the scope of the invention described in the claims.

### EXAMPLES

### Example 1: Association of Declining Klotho Expression with Knee Osteoarthritis

To determine if α-Klotho plays a protective role for the knee joint, the development of knee osteoarthritis in cells having reduced levels of α-Klotho polypeptides, caused by aging or gene knock-down, was evaluated.

### Methods

The knee joints of young (3-6 months old), aged (21-24 months old), young a-Klotho^{+/-} mice (3-6 months, which displayed a reduced expression level of α-Klotho), and aged Klotho mice (21-24 months old) were harvested (n=5 in each group). Normal human articular cartilage tissue from young (15 years old; n=1) and older (69 years old; n=1) donors were collected. Knee joints were decalcified and embedded in paraffin. Safranin-0/Fast green staining and immunofluorescence were performed to evaluate: (i) the cartilage degeneration, (ii) α-Klotho expression level in chondrocytes, and (iii) nuclear morphology of chondrocytes at the knee cartilage. ImageJ and Matlab were used to perform quantification of irregularity of the knee cartilage surface. NIS Elements software was used to quantify Klotho expression per cell. ImageJ was used to quantify nuclear morphology.

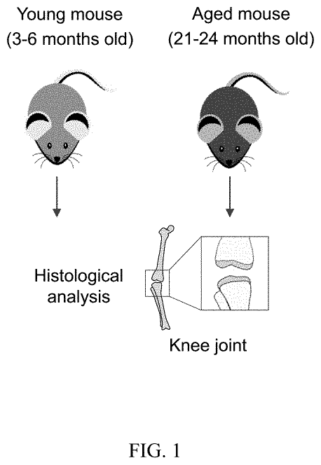

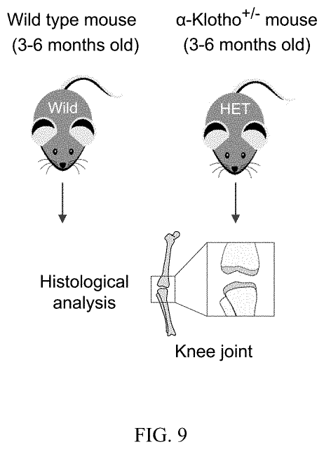

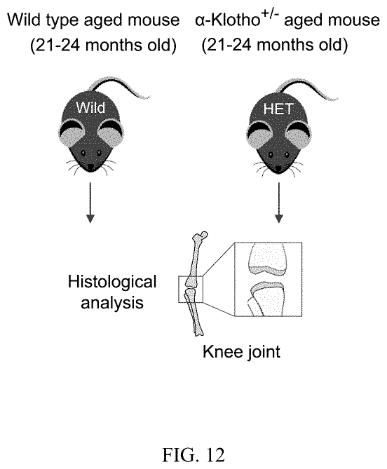

Schematic images of the experimental methods are shown in FIG. 1, FIG. 5, FIG. 9, and FIG. 12.

### Results

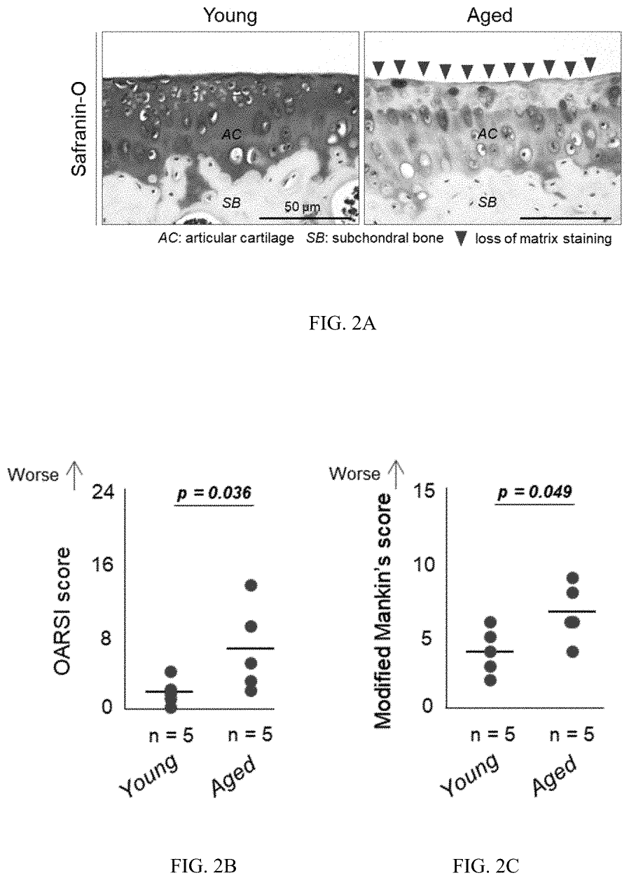

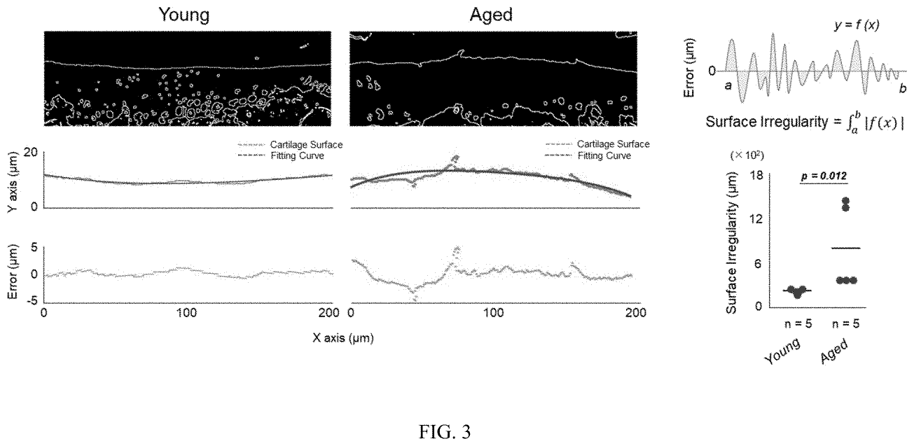

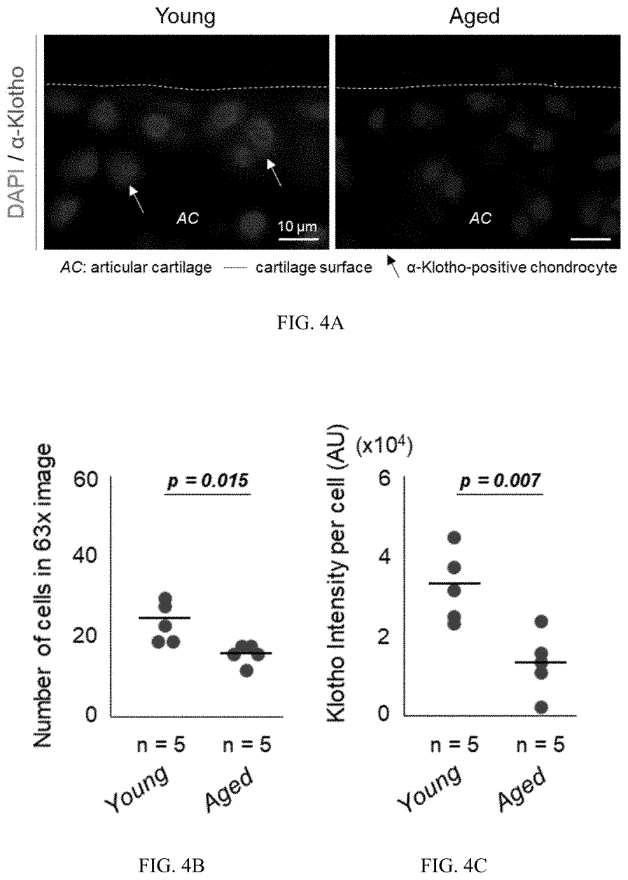

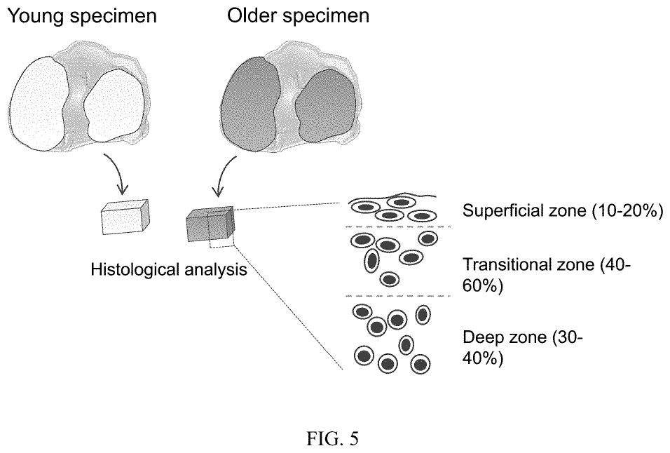

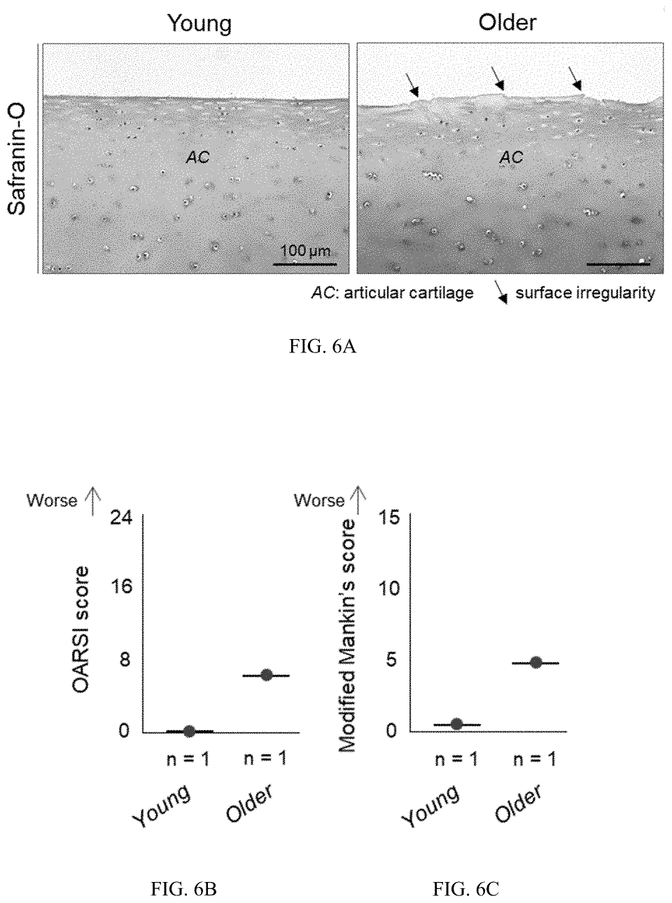

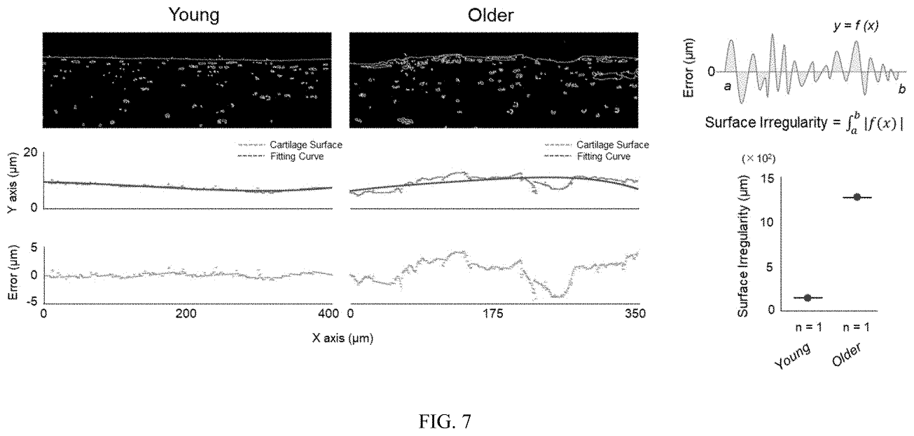

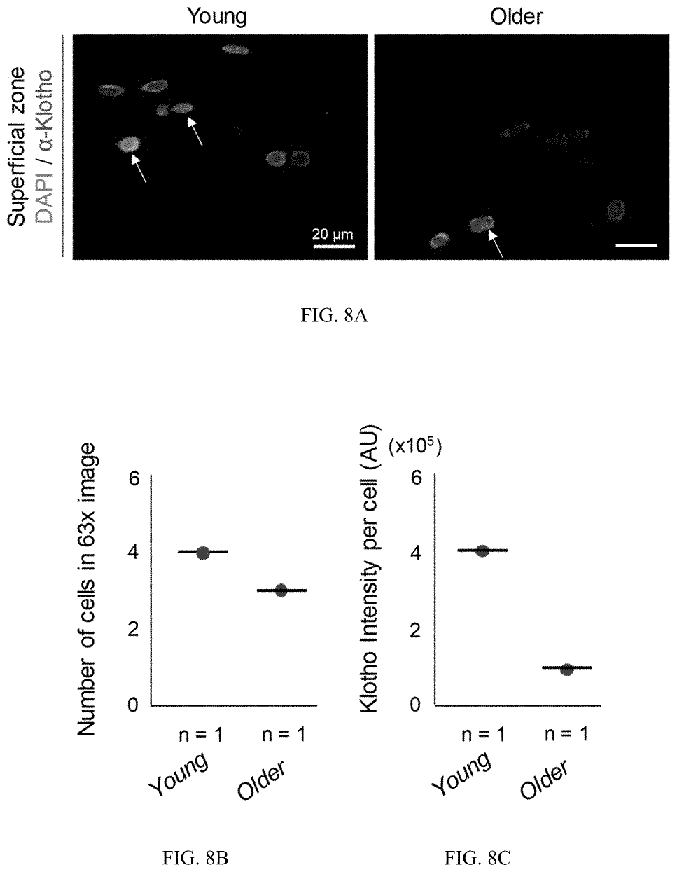

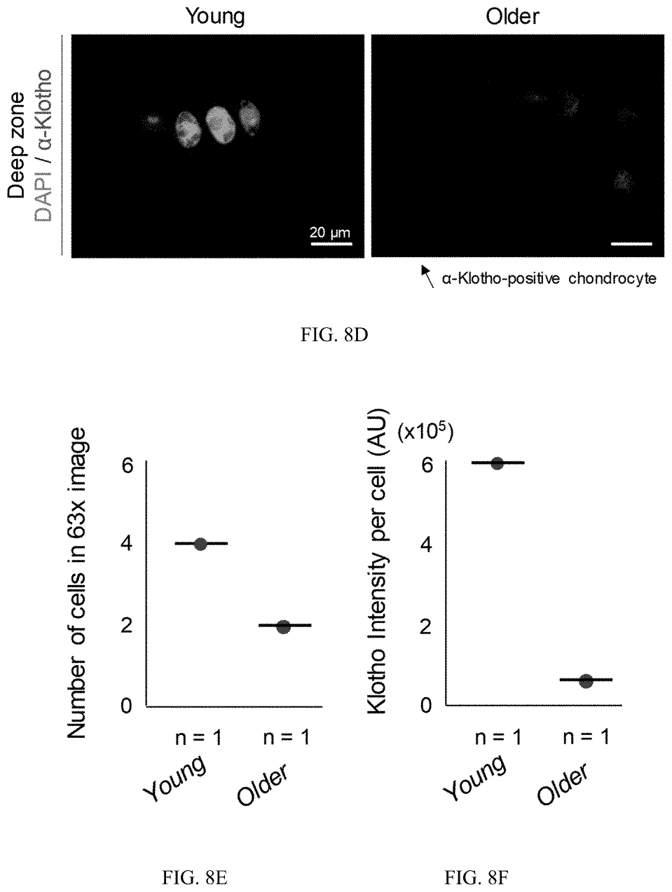

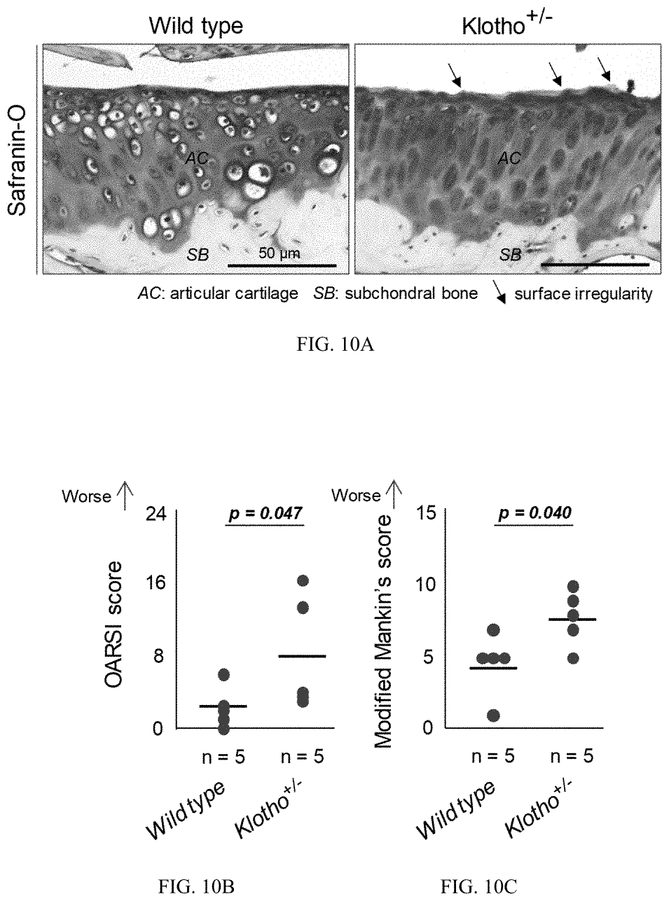

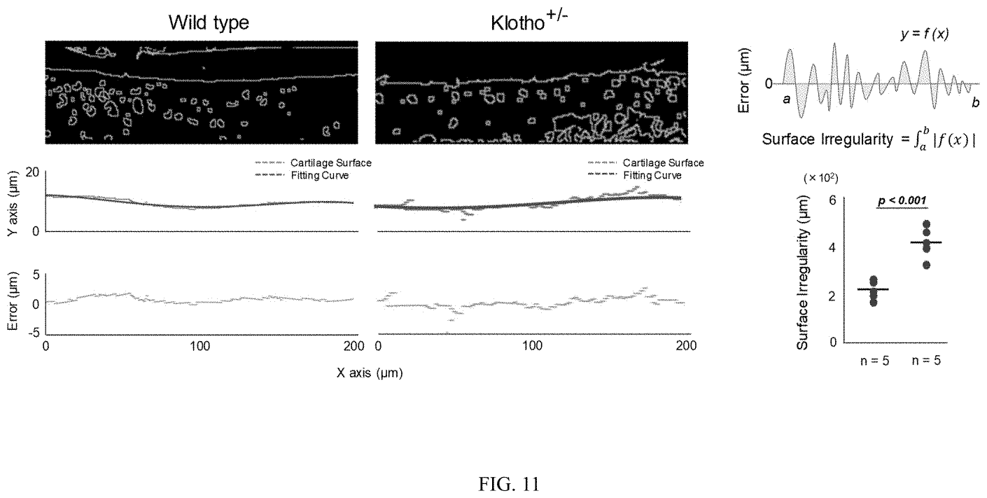

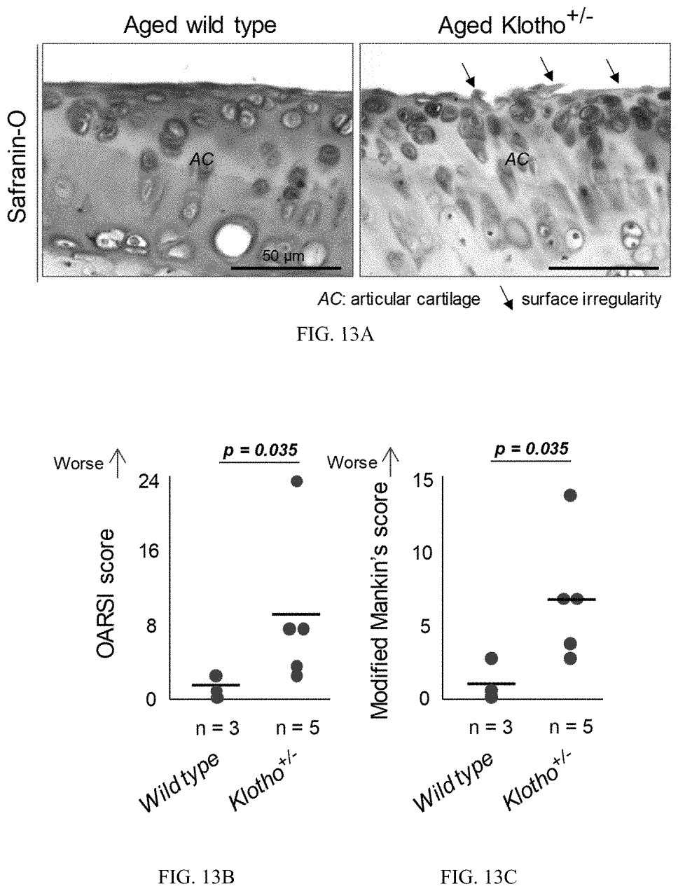

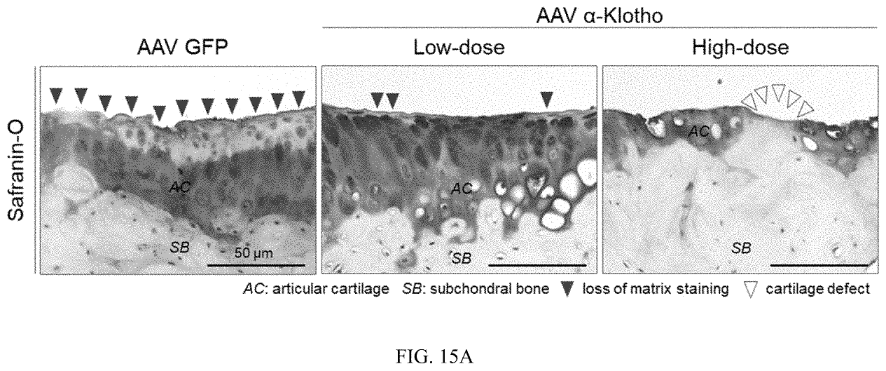

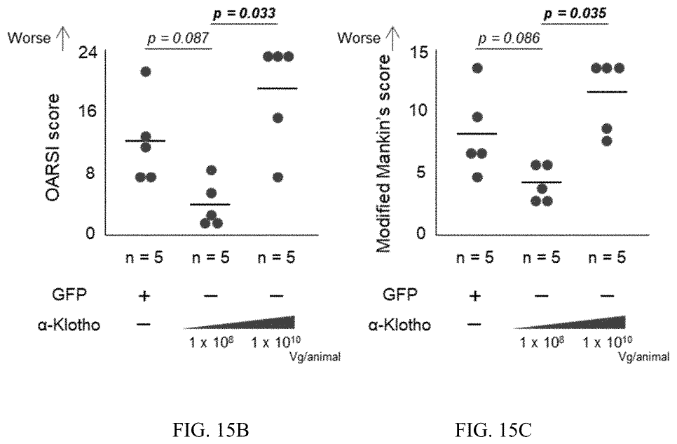

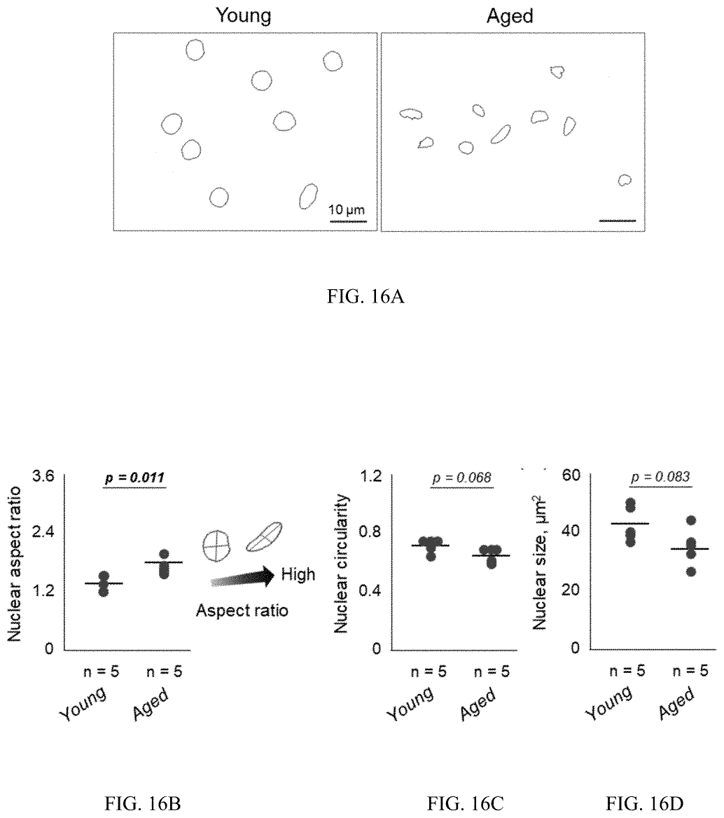

Histology results indicated that aged mice displayed more severe cartilage degeneration (FIG. 2) with increased cartilage surface irregularity (FIG. 3) and altered nuclear morphology (FIG. 15) compared to young mice (FIGS. 2 and 3). Similar results were found in the cartilage from humans (FIGS. 6 and 7). Klotho-signal intensity per cell in aged mice and older donor was significantly lower than that in young mice (FIG. 4) and young donor (FIG. 8), respectively. In addition, the aged-relevant osteoarthritis phenotype was recapitulated in Klotho^{+/-} young mice, as evidenced by an accelerated articular cartilage degeneration (FIG. 10), increased cartilage surface irregularity (FIG. 11), and reduced cellularity (FIG. 10) when compared to age-matched wild type young mice. Furthermore, the age-related cartilage degeneration was accelerated in Klotho^{+/-} aged mice when compared to age-matched wild type aged mice (FIG. 13).

Age-related cartilage degeneration in knee joint was associated with decreased α-Klotho expression in both murine and human models. Genetically engineered mice that express decreased α-Klotho displayed an aged phenotype with the accelerated development of osteoarthritis.

These results demonstrate that a decline in α-Klotho polypeptide levels can contribute to the onset of osteoarthritis.

### Example 2: Exogenous Delivery of α-Klotho

This Example evaluates the ability of α-Klotho to treat knee osteoarthritis. To determine if α-Klotho can be used to treat osteoarthritis in a knee joint, aged mice exhibiting osteoarthritis in the knee join were administered α-Klotho polypeptides.

### Methods

Aged male mice (21 months old) were injected with either a non-targeting control (GFP) or AAV-Klotho with low- (1×10⁸ Vg/animal) and high-dose (1×10¹⁰ Vg/animal) 19 days before euthanasia (n=5 in each group). Knee joints were decalcified and embedded in paraffin. Safranin-O/Fast green staining was performed to evaluate the cartilage degeneration.

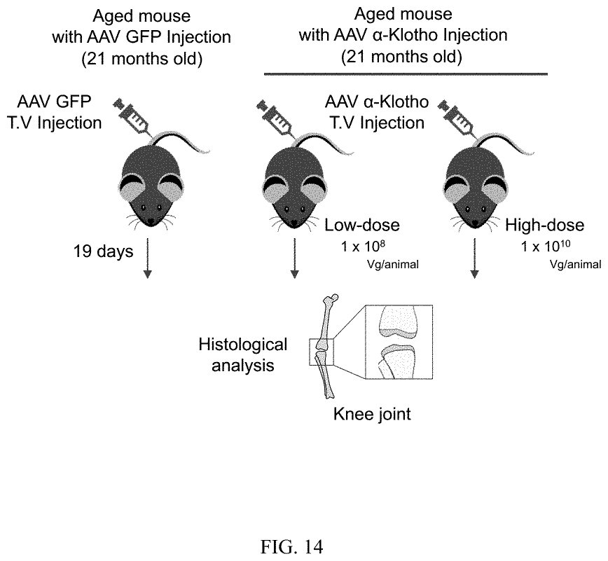

A schematic image of the experimental methods is shown in FIG. 14.

### Results

Histology results indicated that aged mice with AAV-delivery of α-Klotho at a low-dose displayed decreased cartilage degeneration compared to those with AAV-GFP (FIG. 15). These results demonstrate that increasing the level of α-Klotho polypeptides can treat osteoarthritis.

### Example 3—Age-Related Increase in Matrix Stiffness Downregulates α-Klotho in Chondrocytes and Induces Cartilage Degeneration

This example interrogated molecular mechanisms driving age-related KOA in a mouse model and corresponded the findings to human knee cartilage. Unbiased mass spectrometry proteomics of cartilage tissue reveled PI3K/Akt signaling was the predominant pathway disrupted over time in male, but not female, mice. This finding was consistent with a significantly accelerated KOA progression in males when compared to female counterparts. In probing for upstream regulators of these age-dependent alterations, it was found that α-Klotho, a suppressor of PI3K/Akt signaling and potent longevity protein, significantly decreased with aging in both mice and humans. Age-related increases in matrix stiffness initiated a cascade of altered nuclear morphology and downregulated α-Klotho expression, ultimately impairing chondrocyte health. Conversely, decreased matrix stiffness increased α-Klotho expression, enhancing chondrogencity in vitro and cartilage integrity in vivo. These findings establish a mechanistic link between age-related alterations in ECM biophysical properties and regulation of cartilage health by α-Klotho.

### Methods

### Animals

Experiments were performed using young (4-6 months; body mass), middle-aged (10-14 months; body mass), and aged (21-24 months; body mass) male and female C57/BL6 mice, as well as young (4-6 months) and middle-aged (10-14 months) male and female Klotho heterozygotes mice (K^{+/-}; B6; 129S5-Kltm1-Lex, 7-10 months, UC Davis). Mice were obtained from the NIA Rodent Colony and Jackson Laboratories. Prior to inclusion in experiments, animals were evaluated and those with visible health abnormalities were excluded. Mice were housed in cages holding an average of 3-4 mice per cage with a temperature-controlled environment and 12-h light/dark cycles. The animals were able to access food and water ad libitum. All animal experiments were performed in accordance with the ARRIVE guidelines (Kilkenny, et al., PLoS Biol 8, e1000412 (2010)) and approved by the University of Pittsburgh's Institutional Animal Care and Use Committee.

### Human Cartilage Samples

Normal human articular cartilage tissue in knee joint was collected from young (<40 years old; n=3) and older (≥65 years old; n=7) donors with the approval from the Committee for Oversight of Research and Clinical Training Involving Decedents (CORID). Cartilage samples were fixed by 10% buffered formalin phosphate (Fisher Chemical, Fair Lawn, N.J., USA) overnight and dehydrated with 50%, 70%, 95% and 100% ethanol on the second day. After being treated in xylene for 2 hours and then in liquid paraffin overnight, samples were embedded in paraffin. The sample blocks were sectioned at a thickness of 6 μm thickness using a Leica microtome (Model RM 2255). For DAPI staining, sections were deparaffinized using Histo-Clear II (National Diagnostics, Atlanta, Ga., USA), rehydrated, and stained with DAPI mounting medium (Antifade Mounting Medium with DAPI H-1200-10, Vector Laboratories, Burlingame, Calif., USA)

### BAPN Injection of Mice

Aged male C57/BL6 mice (21-24 months; body mass) received daily subcutaneous injections of either saline, as a control, or BAPN (290 μg/μ1 in saline) for 4 weeks every day. Volume of the injection (40-80 μl) was determined based on mice body mass (m) with following protocol: 40 μl (18.45 g≤m≤23.73 g), 50 μl (23.73 g≤m≤29.00 g), 60 ul (29.00 g≤m≤34.27 g), 70 μl (34.27 g≤m≤39.55 g), 80 μl (39.55 g≤m≤44.82 g). Mice were weighed every other day beginning on day 0 and ending on day 28 to ensure proper dosage of BAPN.

### Systemic Administration of α-Klotho Via Osmotic Pumps

Mice were anesthetized with isoflurane. The subscapular implantation area was shaved, and the site was sterilized with 70% ethanol followed by iodine solution. A 1-1.5 cm incision was made between and slightly posterior to the scapula, and a pocket that is 1 cm longer than the osmotic pump was formed under the skin using a hemostat. Care was taken to ensure that the pocket was no larger than necessary to avoid slippage of the pump and also to ensure that the pump did not rest below the incision.

The sterile osmotic pumps (Alzet, mini-osmotic pump, model #2004) were implanted to chronically deliver saline or 324 pg/mL of α-Klotho protein (R&D Systems, Product #aa 35-982) to aged mice. Pumps were inserted subcutaneously at the back of the neck of mice, and the incision was closed with surgical sutures and Rimadyl tablets were given for three days. Mice were monitored daily to ensure that they did not reopen the incision and that there were no signs of infection. They were monitored for postoperative pain or discomfort as evidenced by decreased activity, decreased food and water intake, weight loss, vocalizations, rough hair coat, hunched posture. If signs appear, additional Rimadyl tablets were given. To allow easy access to food, gel food was provided in the first three days. Knee joints were harvested 14 days after the implantation for histological analyses.

### Serum Collection

Serum was collected from animals under anesthesia by isoflurane in accordance with established protocols (Sahu, et al., Nat Commun 9, 4859 (2018)). After collection, the animals were euthanized via cervical dislocation. The collected blood was allowed to clot in a 2 mL tube at room temperature for one-hour, then was centrifuged for 30 minutes at 13,000 rpm at 4° C. in a microcentrifuge. The serum was collected and aliquoted into 50 μL tubes and stored at −20° C. until used. Any samples displaying hemolysis (as evidenced by pink/red coloration) were not included in the analysis.

### Estrogen ELISA

The Estrogen ELISA was conducted according to protocol using the 17 beta Estradiol ELISA Kit (ab108667, Abcam). This kit was validated by Marino et al. (J Cachexia Sarcopenia Muscle 6, 365-380 (2015)). Briefly, 25 μL of blood serum samples, prepared standards, or controls were added in duplicates to a 96 well plate. 200 μL of the 17 beta Estradiol-HRP Conjugate were added to each well. This plate then was incubated at 37° C. for two hours. Samples, standards, and controls were aspirated, and wells were washed three times with 300 μL of diluted washing solution (soak time >5 seconds) using an automated plate washer (BioTek SOTS). After washing, 100 μL tetramethylbenzidine (TMB) Substrate Solution was added to each well, and the plate was incubated for 30 minutes in the dark at room temperature. After incubation, 100 μL of Stop Solution was added into all wells in the same order and at the same rate as the substrate solution. Absorbance was measured at 450 nm with Spectramax M3 plate reader (Molecular Devices) within 30 minutes of adding the Stop Solution. Construction of standard curve and subsequent analyses were performed in Microsoft Excel.

### Histological Preparation and Semi-Quantitative Histological Score for Cartilage Degeneration

Mice knee joints and human cartilage samples were fixed in 4% paraformaldehyde overnight at 4° C., and decalcified in 20% ethylene diamine tetra acetic acid solution for 10 days. Decalcified paraffin sections (5 μm thickness) were prepared from central region of the mice knee joints in the frontal plane in accordance with the OARSI recommendation (Glasson, et al., Osteoarthritis Cartilage 18 Suppl 3, S17-23 (2010)). Decalcified paraffin sections from human cartilage specimen were also prepared. The paraffin sections were stained with Safranin-O/Fastgreen/Hematoxilin to evaluate the severity of cartilage lesions. This study focused on medial tibial cartilage given this is the region most typically affected in humans (Hayashi, et al., Osteoarthritis Cartilage 22, 76-83 (2014)) and its severity is typically equal to or higher than those for the femoral condyle in the majority of aged mice (Armstrong, et al., Osteoarthritis Cartilage, S1063-4584 (2021)). The OARSI scoring system, consisting of six grades and four stages on a scale from 0 (normal) to 24 (severe cartilage lesion), was used for semi-quantitative evaluation of cartilage lesion severity (Pritzker, et al., Osteoarthritis Cartilage 14, 13-29 (2006)). The most severe score was evaluated as the maximum OARSI score. The summed scores generated from different sections were not totaled, as discriminative ability for age-related cartilage degeneration is comparable to the maximum score (Armstrong, et al., 2021, supra). A trained examiner (AB) performed grading in each histological section in a blinded manner.

### Chondrocytes Isolation

Primary chondrocytes were isolated in accordance with an established protocol (Gosset, et al., Nature protocols 3, 1253-1260 (2008)). Briefly, cartilage tissue was harvested from femoral head, femoral condyle, and tibia cartilage using small scissors and tweezers while ensuring that minimal fat, muscle, ligament, and tendons were included in the tissue harvest. After a brief wash by PBS two times, cartilage pieces were digested with 0.1% (w/w) type II collagenase (cat no. 4176, Worthington Biochemical corp., NJ) in low-glucose DMEM (cat no. 11965-092, Gibco) with 10% (vol/vol) FBS (cat no. SH30070.03, Hyclone) and 1% (vol/vol) Pen/Strep at 37° C. for overnight under 5% CO₂ in a petri dish. After the digestion, the filtrate was passed through a 50-μm strainer and cells were culture with growth media containing DMEM supplemented with 10% FBS and 1% Pen/Strep until the cell reached to confluent. First-passage cells were used for all the experiments. The isolated cells were characterized by type II collagen immunofluorescence, and over 95% cells were type II collagen positive.

Preparation of Fibronectin-Coated pAAm Substrates

Poly(acrylamide) (pAAm) gels with different stiffness (5 kPa, 21 kPa and 100 kPa) in accordance with Wang, et al., Nano letters 19, 5443-5451 (2019). The pAAm gels were made on glass coverslips, which are pre-treated with 0.1 N sodium hydroxide (cat. SS255-1, Fisher Scientific, IL), 0.5% 3-aminopropyltrimethoxysilane (cat no. AC313251000, Acros Organics, Belgium) and 0.5% glutaraldehyde (cat no. BP25481, Fisher Scientific, IL) to improve the gel adhesion. See, Table 1. To facilitate the cell adhesion, the surfaces of prepared hydrogels were further conjugated with fibronectin (100 μg/mL, from bovine plasma, Sigma) by using sulfo-SANPAH (cat no. NC1314883, Proteochem Inc., UT) as the crosslinker. Prior to seeding cells, gels were UV-sterilized in cell culture hood for 30 minutes. Gels were kept hydrated in HEPES or PBS during all preparation steps.

TABLE 1
Preparation and characterization of pAAm hydrogels

| | | | | | 10% | |
|---|---|---|---|---|---|---|
| | | 2% | | | Ammo- | |
| | 40% | Bis- | 50 mM | | nium | |
| | Acryl- | acryl- | HEPES | TEME | per- | Substrate |
| | amide | amide | (μl) | D | sulfate | stiffness |
| pAAm | (μl) | (μl) | (pH 8.2) | (μl) | (μl) | (kPa)* |
| 5 kPa | 62.5 | 25 | 412.5 | 1.5 | 5 | 4.9 ± 0.5 |
| (soft) | | | | | | |
| 21 kPa | 150 | 25 | 325 | 1.5 | 5 | 21.2 ± 0.5 |
| (medium) | | | | | | |
| 100 kPa | 150 | 125 | 225 | 1.5 | 5 | 100.8 ± 2.1 |
| (stiff) | | | | | | |

*The Young's modulus of pAAm gels was measured by using atomic force microscopy.

### Cell Fate Influenced by Matrix Stiffness

Isolated primary chondrocytes from young and aged mice were plated (5000 cells per mm²) on 5 kPa, 21 kPa, and 100 kPa pAAM gel and cultured in low-glucose DMEM supplemented with 10% FBS and 1% Pen/Strep at 37° C. under 5% CO₂. At day 3, the culture medium was removed and fixed by 4% PFA for 10 minutes. After a triple wash by PBS, cells were kept in PBS at 4° C. until used.

### Immunofluorescence and Imaging

Immunofluorescence analysis for tissue section and/or cell-seeded pAAm gel was performed to determine the signal intensity of type II collagen, α-Klotho, Lamin A/C, and Lamin B2 in accordance with established protocol (Sahu, et al., 2018, supra). Briefly, after a triple wash by PBS, cells were permeabilized with 0.1% triton-X (Fluka 93420) for 15 minutes, followed by a one-hour blocking step using 0.1% triton-X with 3% Bovine Serum Albumin (BSA, Sigma A7906) in PBS. The cells were then incubated with primary antibodies for overnight at 4° C. A similar process was followed for the decalcified tissue paraffin. For antigen-retrieval for the decalcified tissue paraffin section, they were incubated in sodium citrate buffer for 2 hours at 60° C. before the blocking step. After the blocking step, the tissue or cells were incubated overnight at 4° C. with the primary antibodies in antibody solution (0.1% Triton-X+3% BSA+5% Goat Serum), at various dilutions as indicated in Table 2. One negative control slide per staining set was generated by deleting the primary antibody in the antibody solution.

TABLE 2
Information and dilution protocol of antibodies

| Primary antibodies | Host-species | Product number | Dilution |
|---|---|---|---|
| Type II collagen | Rabbit | Ab34712, Abcam | 1:200 |
| α-Klotho | Rat | MAB1819, R&D systems | 1:100 (tissue), |
| | | | 1:200 (cell) |
| Lamin A/C | Mouse | Sc-376248, Santa Cruz | 1:50 (tissue), |
| | | Biotechnology | 1:100 (cell) |
| Lamin B2 | Rabbit | Ab151735, Abcam | 1:50 (tissue), |
| | | | 1:100 (cell) |

After a triple wash by PBS, the samples were incubated with host-specific secondary antibodies conjugated with Alexa Fluor 488 (Fisher Scientific) in antibody solution for one hour at room temperature at the dilutions of 1:500. Following a triple wash with PBS, the samples were stained with DAPI for 2 minutes and then washed with PBS again. The samples were mounted with coverslips using Gelvatol mounting medium (Source: Center of Biologic Imaging, University of Pittsburgh). The antibody of α-Klotho antibody (R&D systems, MAB1819, Lot #KGN0315101) was validated for skeletal muscle histological section in Sahu, et al., 2018, supra. The same α-Klotho antibody also was validated using the decalcified paraffin section from a wild type and a Kl−/− mouse knee joint. Minimal background staining was observed in the Kl−/− section as compared to the wild-type counterparts.

Slides were imaged using a Zeiss Observer Z1 semi-confocal microscope. All images were collected at 20× or 63× magnification. Negative control slides were used to threshold for the signal intensity and to set the exposure time for individual channels. All images for quantitative analysis in a given experiment were taken under the same imaging conditions. Fluorescence intensity was quantified using Image J

### Quantification of Cellular and Nuclear Morphology

DAPI and F-actin images were obtained at 63× and 20× magnification using a Zeiss Observer Z1 semi-confocal microscope, respectively. Afterwards, image processing and morphome feature extraction were performed using CellProfiler software (v4.0, The Broad Institute) (Carpenter et al., Genome Biol 7, R100 (2006)). Fifty-three shape features of cell and nuclei were determined using the “identify primary objects” followed by the “measure object size shape” and “export to spread sheet” module. Principal Component Analysis (PCA) was performed for the data reduction identifying the principal components (PCs) that represent the differences in the cellular and nuclear morphology. To determine variables of cellular and nuclear shape contributing to PCs, loading matrix, a correlation between the original variable and PCs, were extracted.

### Gene Set Enrichment Analysis

Single sample gene set enrichment analysis (ssGSEA) was performed by Gene Set Enrichment Analysis software (software.broadinstitute.org/gsea/index.jsp). Gene scores defined by absolute value of log 2 fold change of gene expression profiles after treatment of siRNA Klotho and recombinant α-Klotho protein supplementation as described elsewhere (see, e.g., Chuchana et al., Aging (Albany, N.Y.), 10:1442-1453 (2018)) were used as an input variable. PI3K/Akt pathway involved in KEGG pathways (KEGG_2019_Mouse) were used as a gene set downloaded from enrichr (amp.pharm.mssm.edu/Enrichr/).

### LC/MS-MS Mass Spectrometry Proteomics

Knee cartilage from male and female young, middle-aged, and aged C57/BL6 mice were microdissected as detailed by Gardiner, et al., Osteoarthritis Cartilage 23, 616-628 (2015). Briefly, cartilage tissue was harvested from femoral condyle and tibial plateau using small scissors and tweezers while ensuring that minimal fat, muscle, ligament, and tendons were included in the tissue harvest. Immediately after dissection, cartilage samples were washed with PBS. Samples were then lyophilized over-night and stored at −80° C. until shipment to the Proteome Exploratory Laboratory at Cal Tech.

Cartilage samples from each knee were lysed in 150 μL 8M urea/100 mM TEAB by grinding for 1 min with 0.5 mL size tissue grinder pestles (Fisher Scientific #12141363), tip sonication with a Fisher Scientific 550 Sonic Dismembrator on ice at 20% power using cycles of 20 sec on/20 sec off for 4 minutes total, followed by another grinding step for 1 min. Samples then were clarified by centrifugation at 16,000 g for 5 minutes at room temperature and the lysate collected for protein quantitation by BCA assay (Pierce). Each lysate was reduced with 1.12 μL 500 mM TCEP for 20 min at 37° C. and alkylated with 3.36 μL 500 mM 2-Chloroacetamide for 15 min at 37° C. in the dark. Samples were then digested with a 1:200 ratio of LysC to lysate for 4 hr at 37° C., followed by dilution with 450 μL 100 mM TEAB, addition of 6 μL 100 mM CaCl₂), and digestion overnight (16 hr) with 1:30 Trypsin at 37° C. Digestions were stopped by acidifying with 20 μL 20% TFA, desalted on C18 spin columns (Pierce #89870) according to manufacturer instructions, and lyophilized to dryness. Peptides were then resuspended in 50 μL 0.1% formic acid and peptide amounts measured with the Pierce Quantitative Colorimetric Peptide Assay.

15 μg peptides from each sample were lyophilized, resuspended in 50 μL 100 mM TEAB, labelled with 0.25 mg TMTpro reagents dissolved in 10 μL anhydrous acetonitrile for 1 hr at room temperature, and quenched with 2.5 μL 5% hydroxylamine for 15 min at room temperature. Concurrently, a single 30 μg pooled bridging control sample was created by mixing 1 μg from each of the 30 samples into a single tube which was then lyophilized, resuspended in 100 μL 100 mM TEAB, labelled with 0.5 mg TMTpro-134 dissolved in 20 μL anhydrous acetonitrile for 1 hr at room temperature, and quenched with 5 μL 5% hydroxylamine for 15 min at room temperature. All 15 male samples were then combined into one sample and all 15 female samples were combined into a second sample. Half of the labelled bridging control sample was then mixed into each and the two resulting mixed samples were lyophilized and stored at −20° C. 100 μg of each sample was then fractionated with the Pierce High pH Reversed-Phase Peptide Fractionation Kit (Thermo #84868) according to manufacturer instructions and the resulting 8 fractions were lyophilized. Each fraction was resuspended in 20 μL 0.2% formic acid and peptide quantitation performed with the Pierce Quantitative Colorimetric Peptide Assay. Fractions 7 and 8 from both samples had very low peptide amounts and were thus combined with that sample's fraction #6 for a total of 6 fractions per sample.

Liquid chromatography-mass spectrometry (LC-MS) analysis of peptide fractions was carried out on an EASY-nLC 1000 coupled to an Orbitrap Eclipse Tribrid mass spectrometer (Thermo Fisher Scientific). For each fraction, 1 μg peptides were loaded onto an Aurora 25 cm×75 μm ID, 1.6 μm C18 reversed phase column (Ion Opticks, Parkville, Victoria, Australia) and separated over 136 min at a flow rate of 350 nL/min with the following gradient: 2-6% Solvent B (7.5 min), 6-25% B (82.5 min), 25-40% B (30 min), 40-98% B (1 min), and 98% B (15 min). MS1 spectra were acquired in the Orbitrap at 120K resolution with a scan range from 350-1800 m/z, an AGC target of 1e6, and a maximum injection time of 50 ms in Profile mode. Features were filtered for monoisotopic peaks with a charge state of 2-7 and a minimum intensity of 2.5e4, with dynamic exclusion set to exclude features after 1 time for 45 seconds with a 5-ppm mass tolerance. HCD fragmentation was performed with collision energy of 32% after quadrupole isolation of features using an isolation window of 0.5 m/z, an AGC target of 5e4, and a maximum injection time of 86 ms. MS2 scans were then acquired in the Orbitrap at 50K resolution in Centroid mode with the first mass fixed at 110. Cycle time was set at 3 seconds.

Analysis of LCMS proteomic data was performed in Proteome Discoverer 2.5 (Thermo Scientific) utilizing the Sequest HT search algorithm with the mouse proteome (UniProt UP000000589; 55,485 proteins covering 21,989 genes with a BUSCO assessment of 99.8% genetic coverage). Search parameters were as follows: fully tryptic protease rules with 2 allowed missed cleavages, precursor mass tolerance set to 20 ppm, fragment mass tolerance set to 0.05 Da with only b and y ions accounted for. Modifications were considered static for TMTpro (K, N-term), with dynamic modifications considering Loss of Protein N-term Methionine, Acetyl (N-term), Oxidation (M), Carbamidomethyl (C) and Phosphoration (S, T). Percolator was used as the validation method, based on q-value, with a maximum FDR set to 0.05. GO Biological Process terms were generated via Proteome Discoverer Sequest HT algorithm.

Quantitative analysis is based on TMT MS2 reporter ions generated from HCD fragmentation, with an average reporter S/N threshold of 10, used a co-isolation threshold of 50 with SPS mass matches set at 65%. Normalization was performed at the peptide level, and protein ratios were calculated from the grouped ion abundances, with protein FDR set to a maximum of 0.05.

The mass spectrometry proteomics data have been deposited to the ProteomeXchange Consortium via the PRIDE partner repository (Perez Riverol, et al., Nucleic Acids Res 47, D442-d450 (2019)) with the dataset identifier PXD024062 and 10.6019/PXD024062.

Kyoto Encyclopedia of Genes and Genomes pathway analyses were performed using ROnToTools R Code (Voichita and Draghici, R package version 2.18.0. (2020)). This program was chosen for its ability to integrate both over-representation analyses and functional class scoring. The code was used as written, with the exception of changing ‘hsa’ to ‘mmu’ such that mouse pathways were referenced. Dataset matrices were generated in Excel following the format provided in the sample dataset.

### Statistical Analysis

All statistical analyses were performed using JMP Pro 14 software (SAS Institute, Cary, N.C.). The data are displayed as means, with uncertainty expressed as 95% confidence intervals (mean±95% CI). Linear regression analyses or two-way anova were performed. We checked the features of the regression model by comparing the residuals vs. fitted values (i.e., the residuals had to be normally distributed around zero), and independence between observations. No correction was applied for multiple comparison because that these outcomes determined priori and outcomes were highly correlated. Any statistical analyses considered confounder (e.g., body mass) because of small sample size. We conducted complete-case analysis in the case of missing data. In all experiments, p-values <0.05 were considered statistically significant. Throughout this text, “n” represents the number of independent observations of knees or cells from different animals.

### Methodological Rigor

This study was conducted according to the ARRIVE essential 10 (du Sert, et al., PLoS biology 18, e3000410 (2020)). Where possible, power analysis from pilot data were done to select the number of animals needed for the study using Power and Sample Size Program (version 3.1.2; Vanderbilt University Medical Center, Nashville, Tenn.) (Dupont and Plummer, Jr., Control Clin Trials 19, 589-601 (1998)). For example, sample size calculation estimated that 10 mice were required to get the statistical power of 0.8 based on the histology score from aging cohort (n=5 in male young and male aged mice). In addition, a priori power analysis for mass spectrometry estimated that five mice in each group give us a statistical power of 0.90 when a two-fold change is detected, assuming 20% variation (Levin, Proteomics 11, 2565-2567 (2011). For in vivo experiments such as BAPN injection, animals were randomly allocated into control and treatment groups using a computer-generated randomization. Treatment and placebo treatment were conducted in the same condition and the order of treatment was randomly performed. The cage location was randomly replaced to prevent any bias from environment. All histological outcome assessment were conducted in a blinded manner.

### Results

### Aging Induces Accelerated Cartilage Degeneration in Males Relative to Female Counterparts

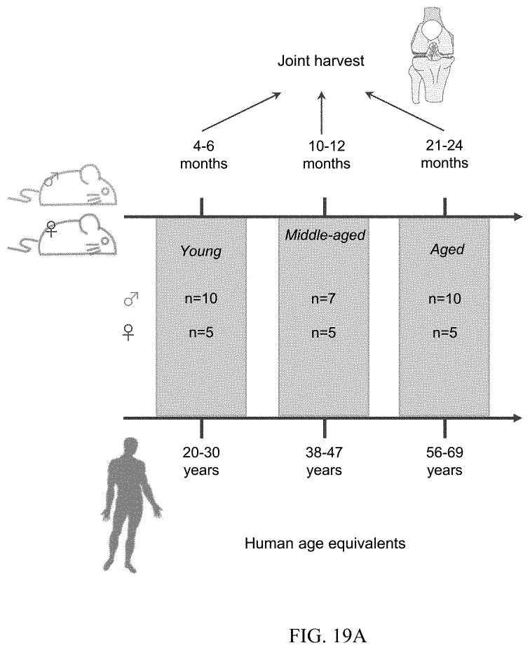

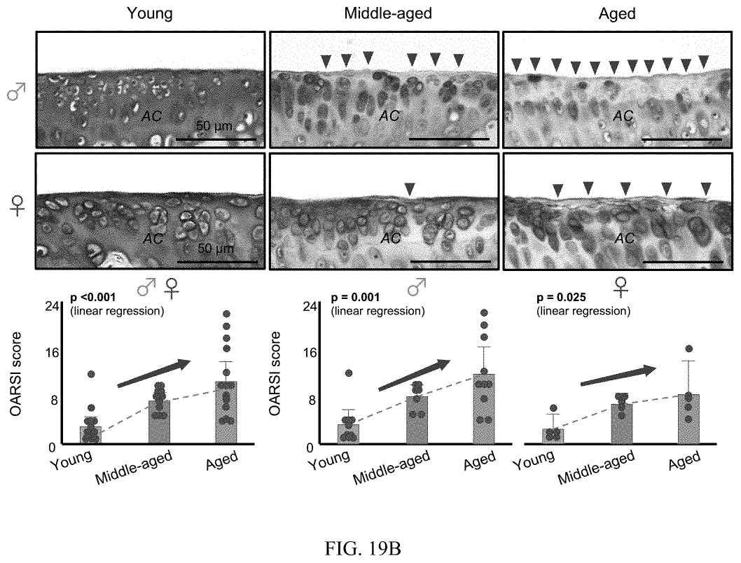

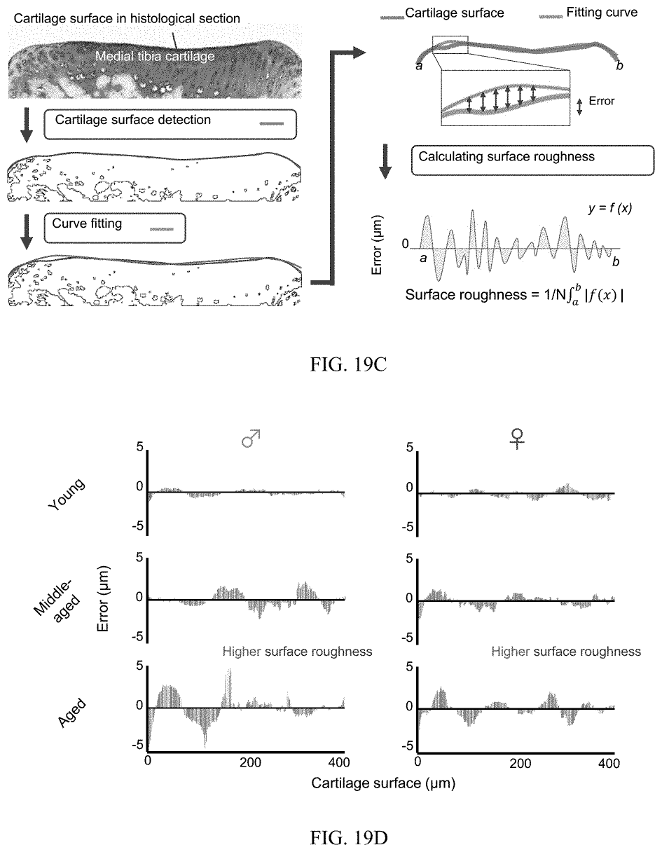

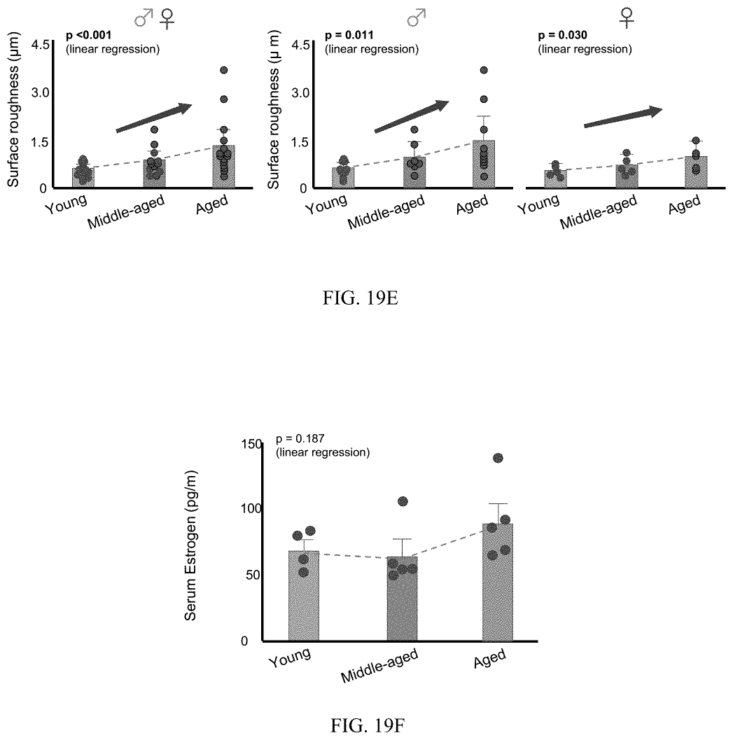

To characterize the natural trajectory of age-associated KOA in mice, cartilage integrity was evaluated in three age groups of male and female C57/BL6 mice: young (4-6 months), middle-aged (10-12 months), and aged (18-24 months) (FIG. 19A). These age groups correspond to 20-30, 38-47, and 56-69 years of age in humans, respectively. This study focused on medial tibial cartilage given this is the region most typically affected in humans (Hayashi, et al., Osteoarthritis Cartilage 22, 76-83 (2014)).

Histological findings confirmed progressive cartilage degeneration beginning at middle-age. These findings are in line with clinical reports of structural abnormalities that manifest in human cartilage between the ages of 45-55 years old (Kumm, et al., Acta orthopaedica 89, 535-540 (2018) (FIG. 19B). To further support the progressive cartilage degeneration, we quantified cartilage surface roughness (FIG. 19C) and found that surface roughness increased with aging (FIG. 19D, E). Moreover, the magnitude of cartilage degeneration and increased surface roughness was greater in male mice than in female mice (FIG. 19D, E). It is unclear why female mice appear to display relative protection against KOA. However, it is worth noting that serum estrogen levels were unchanged across the three female age groups, indicating that aged female mice do not experience the same menopausal phenotypic changes seen in humans (FIG. 19F). Previous studies have demonstrated that approximately 65% of aged female mice spontaneously transition to a polyfollicular anovulatory state, with estrogen and progesterone profiles resembling that of premenopausal women (Finch, et al., Endocr Rev 5, 467-497 (1984)). This is especially important since post-menopausal women typically present with more severe KOA than men (Srikanth, et al., Osteoarthritis Cartilage 13, 769-781 (2005)).

### Mass Spectrometry Proteomics Reveal Age-Related Enrichment of PI3K-Akt Signaling Pathway in Male, but not Female, Mice

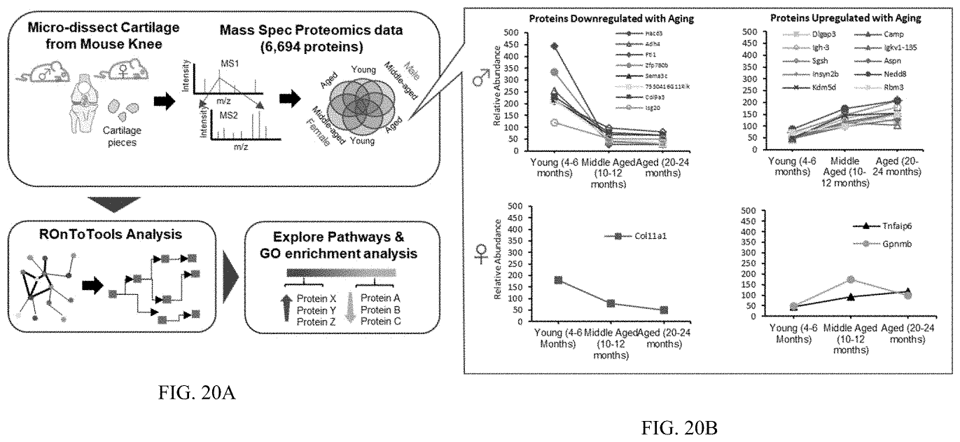

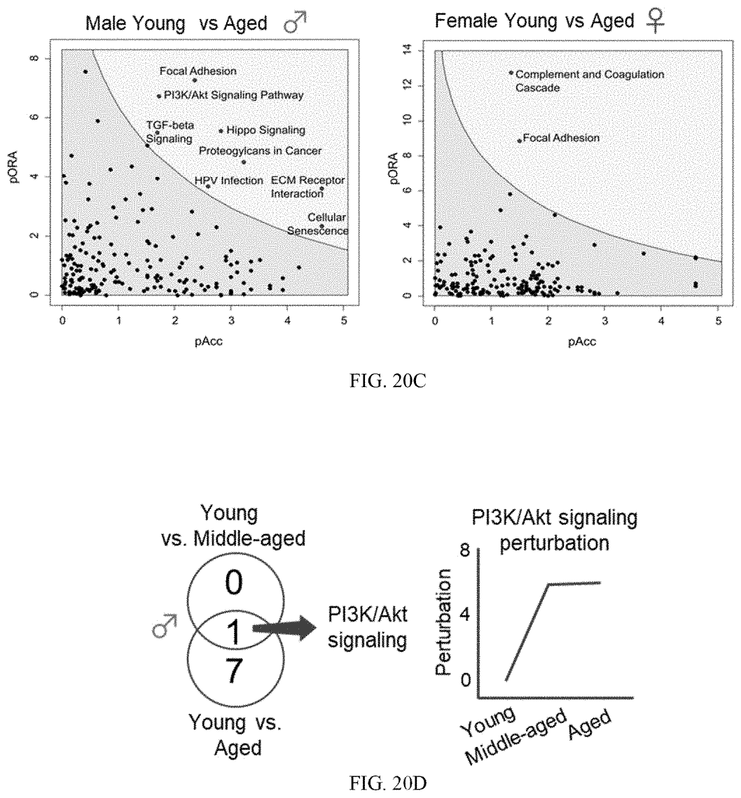

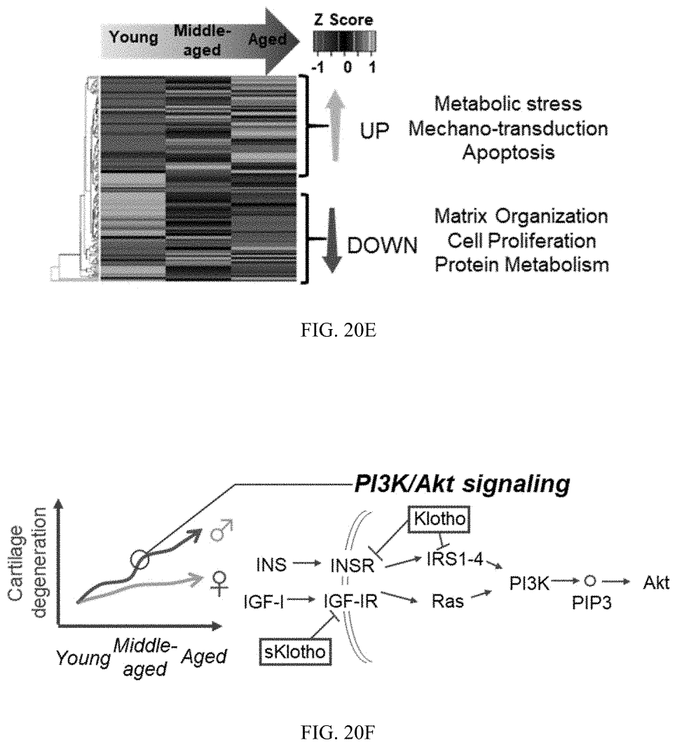

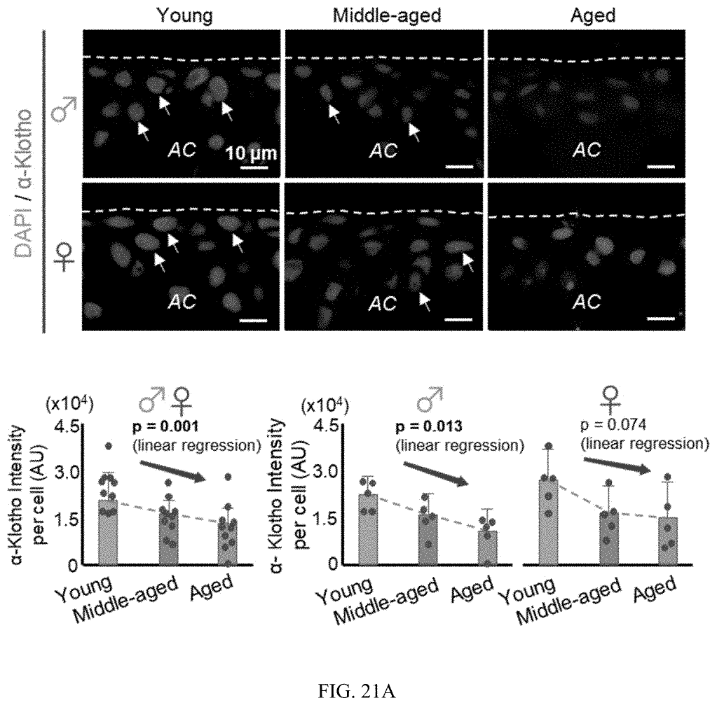

Mass spectrometry proteomics next was assessed to explore potential signaling pathways associated with sex- and age-dependent cartilage degeneration. Articular cartilage was collected from young, middle-aged, and aged male and female mice (n=5/age/sex; FIG. 20A). From these samples, 44,689 peptides associated with 6,694 unique proteins (FIG. 20A) were identified. Data are available via ProteomeXchange with identifier PXD024062 (see Methods for details).

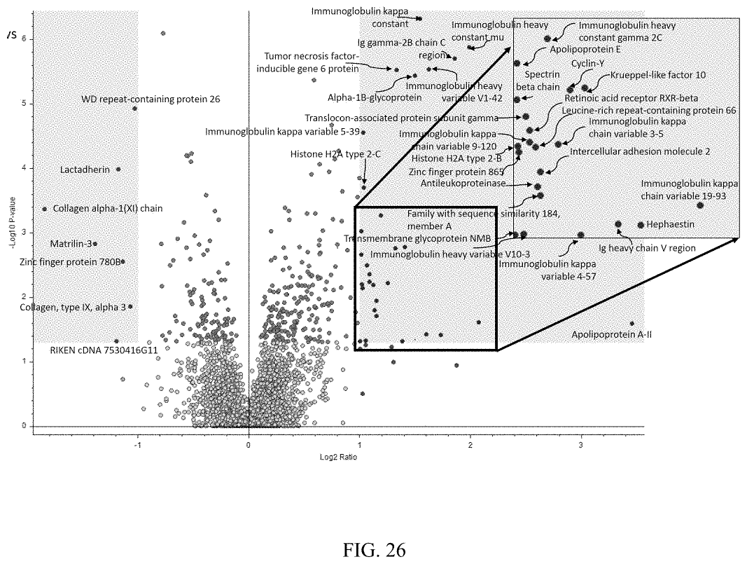

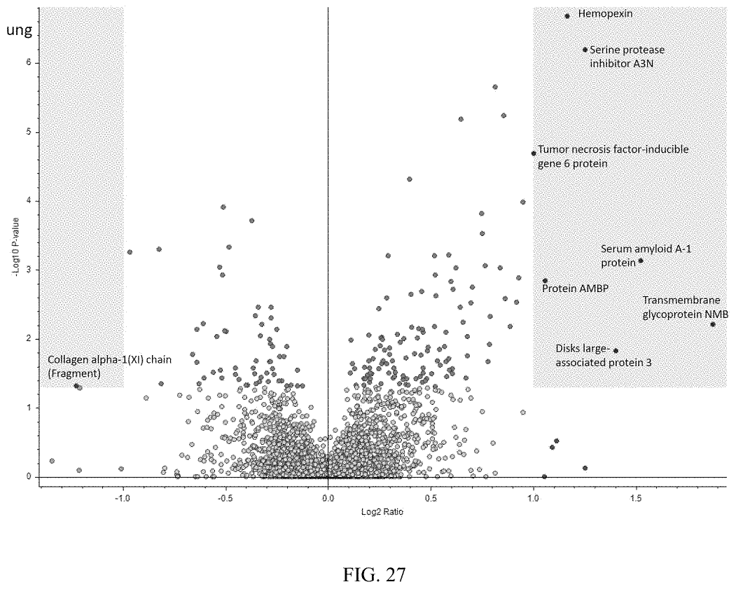

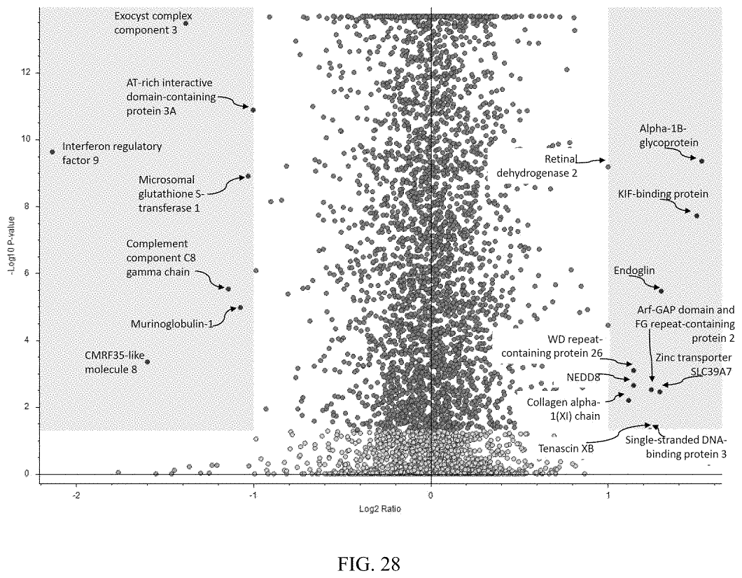

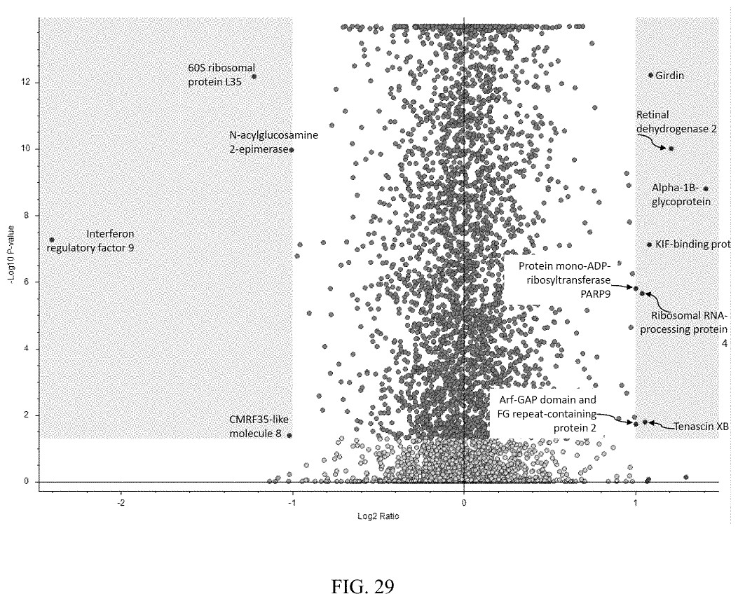

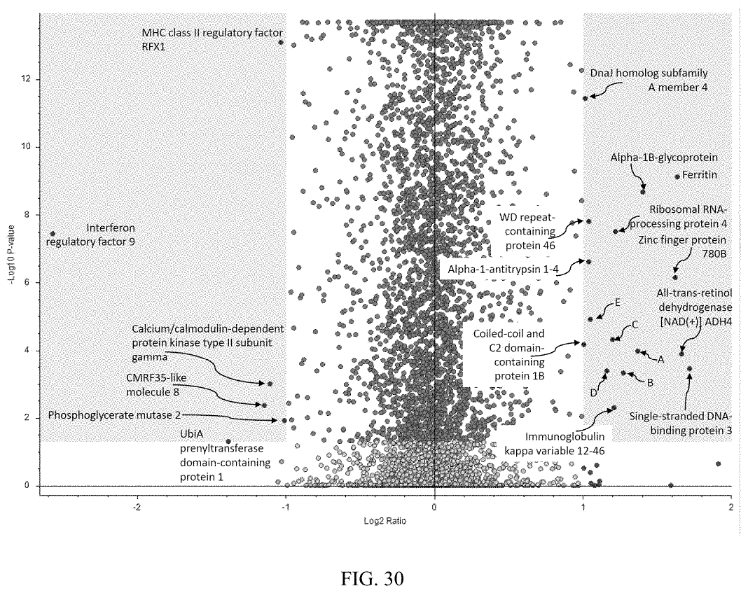

Nine proteins were significantly different between young and middle-aged female samples, and thirty-eight proteins were significantly different when comparing young and aged female cartilage (FIGS. 26 and 27). Of these, only three proteins were significantly different in both the young vs. middle-aged and young vs. aged comparisons (FIG. 2B). A similar comparison was performed between sexes (male vs. female) across all three age groups and is shown in FIGS. 28-30. These analyses revealed prominent changes occurred in immune related proteins, suggesting that differences in immune function may play a role in the observed sex differences.

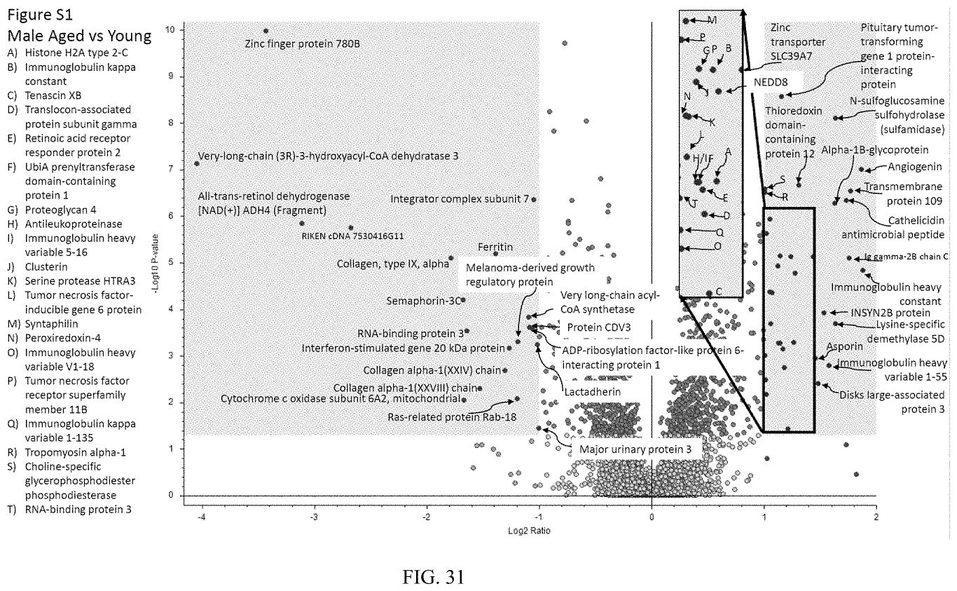

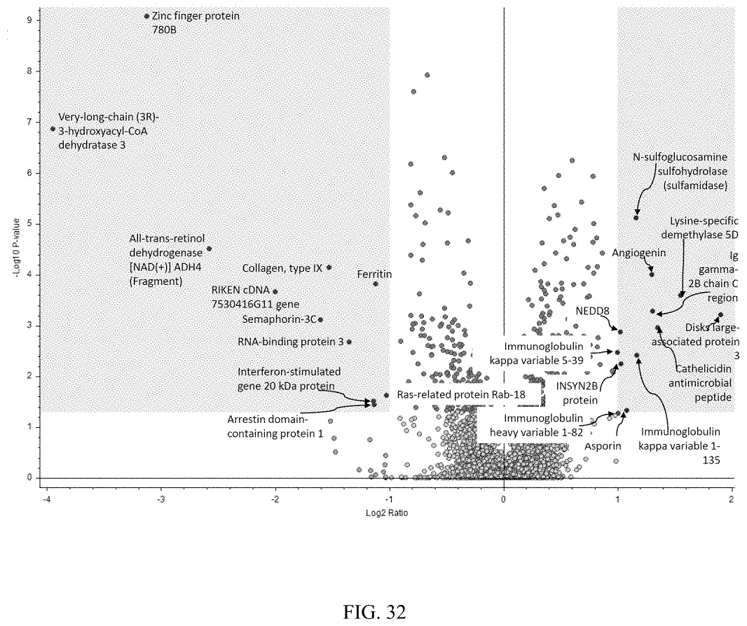

Age-associated trends in male mice were notably different when compared to female counterparts. Twenty-three proteins were significantly different between young and middle-aged male mice, and fifty-six proteins were significantly different between young and aged male mice (FIGS. 31 and 32). Of these, 20 differentially expressed proteins were common to both the young vs. middle-aged and young vs. aged male comparisons. Compared to female mice, male mice demonstrate far more extensive changes in protein content with aging, suggesting distinct sex-related differences in cartilage-aging phenotype. Interestingly, the greatest magnitude of protein changes were observed between the young and middle-aged samples (FIG. 20B).

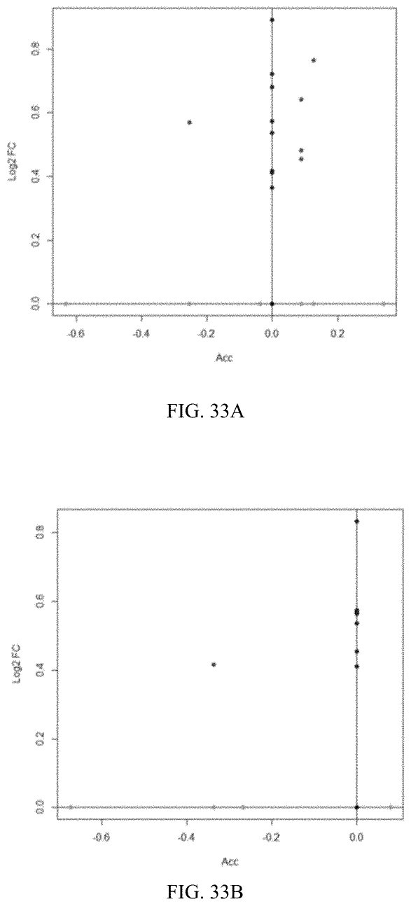

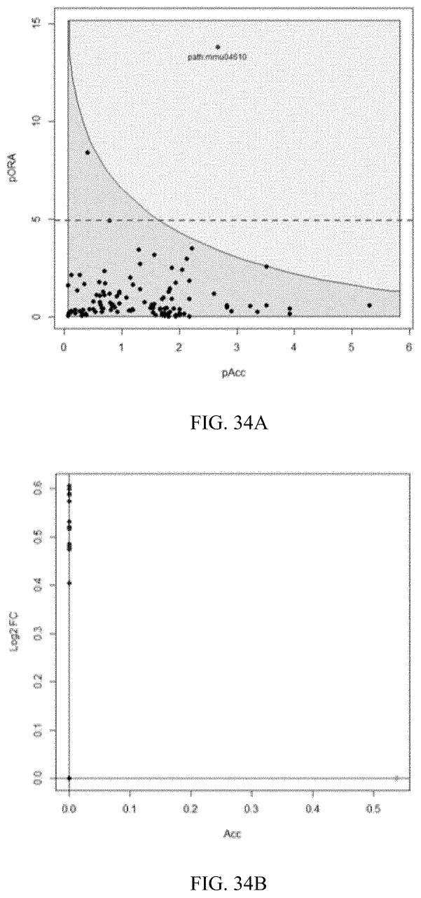

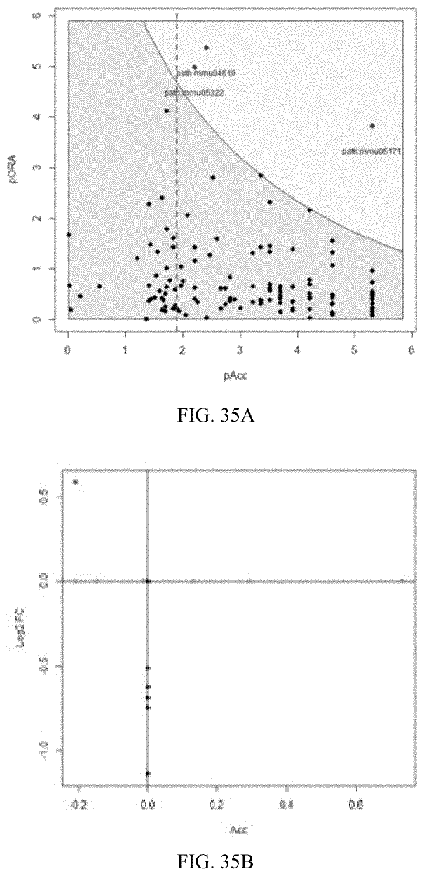

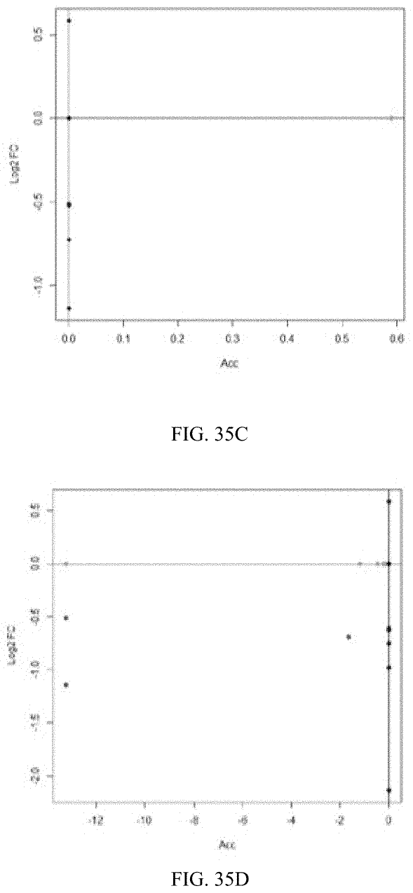

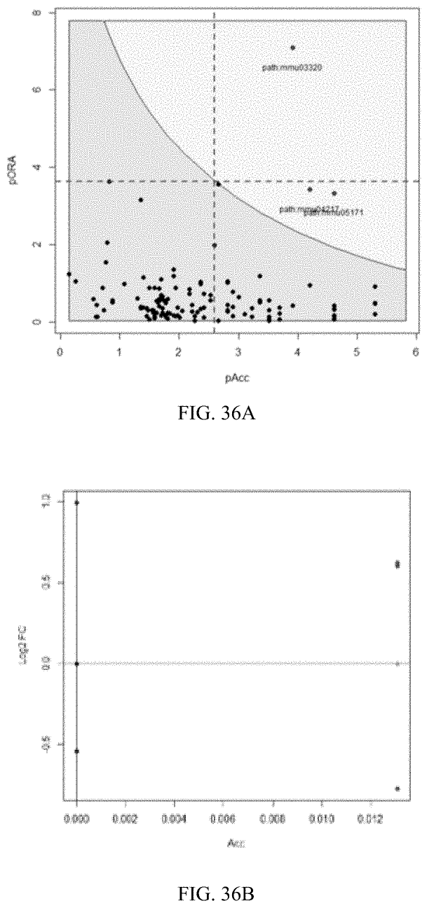

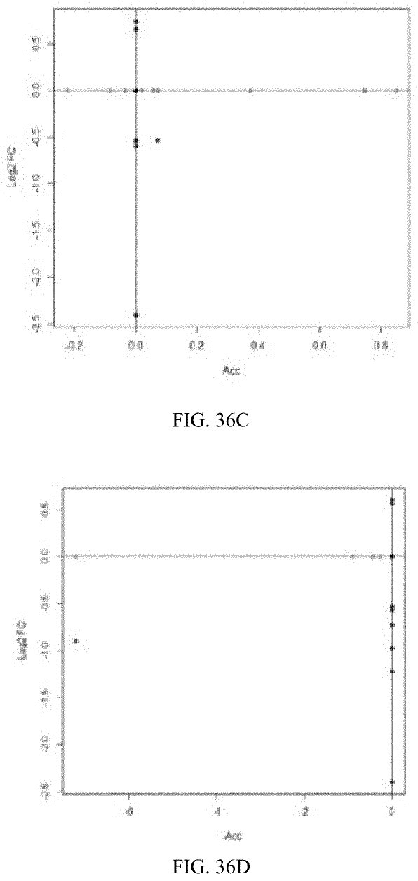

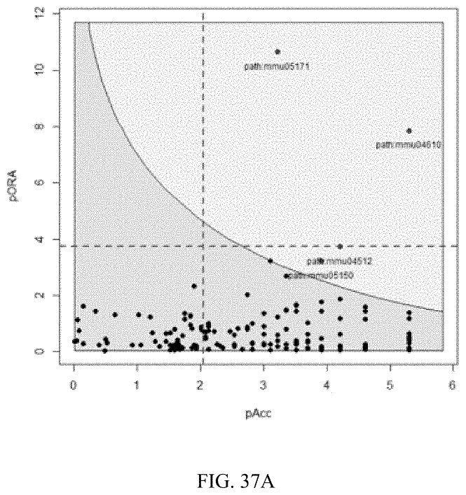

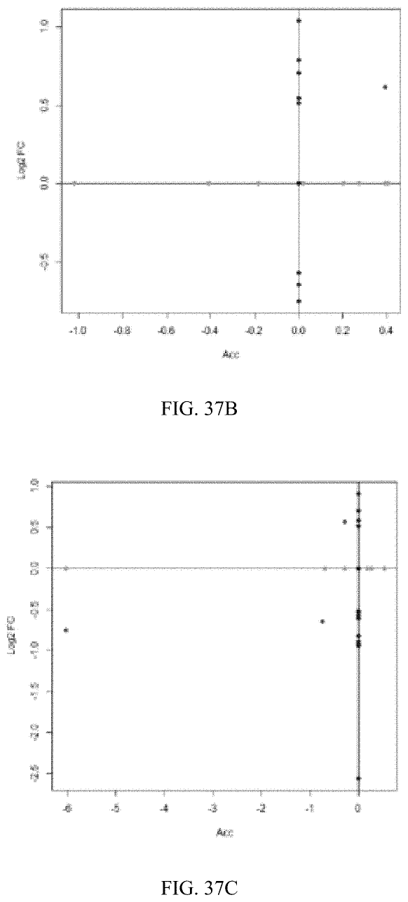

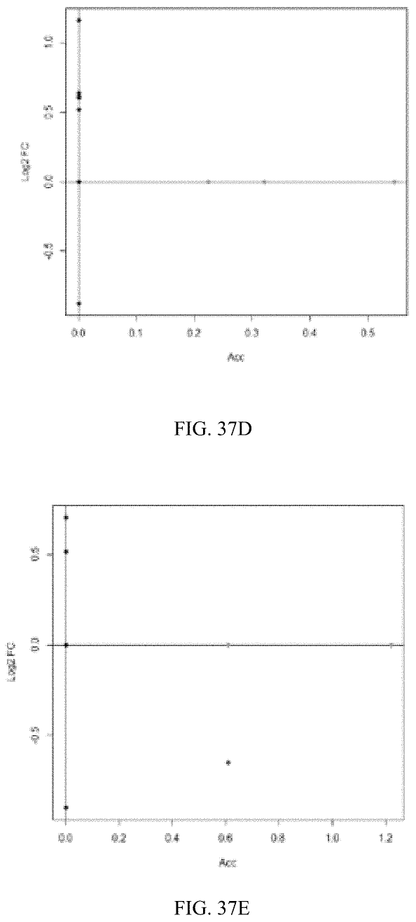

Next, Kyoto Encyclopedia of Genes and Genomes (KEGG) enrichment analyses were performed to gain a holistic understanding of the protein network level changes in knee cartilage with aging (Nguyen, et al., Genome biology 20, 203 (2019)). As suggested by the individual protein data, few pathways changed over time in female mice, with only one significantly enriched pathway between young and middle-aged samples and only two pathways differentially expressed between young and aged samples (FIG. 20C & FIGS. 33 and 34). Of these, the Coagulation and Complement Cascade changed in both the young vs. middle-aged and young vs. aged comparisons. The complement cascade is activated in OA through mechanical, catabolic, and inflammatory mediators (Silawal, et al., Clinical medicine insights. Arthritis and musculoskeletal disorders 11, 1179544117751430 (2018)), and studies have shown complement is elevated in human joints with OA (Wang, et al., Nature medicine 17, 1674-1679 (2011)). A similar pathway analysis was performed comparing sexes (male vs. female) across the three age groups and is shown in FIGS. 34-36. Similar to the individual protein analysis, pathway analyses between sexes revealed significant enrichment of immune related processes.

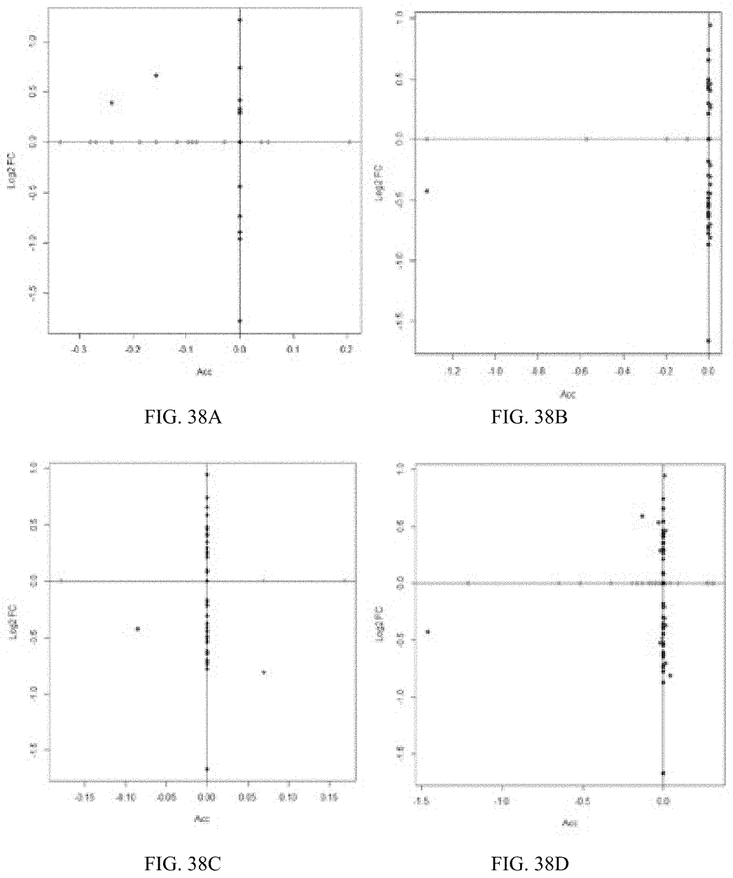

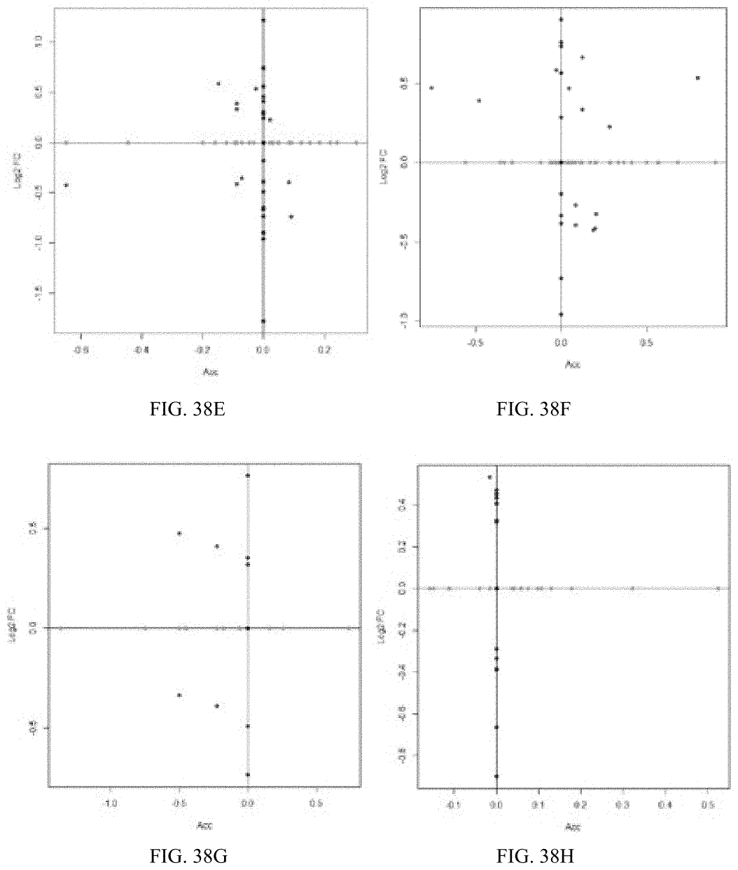

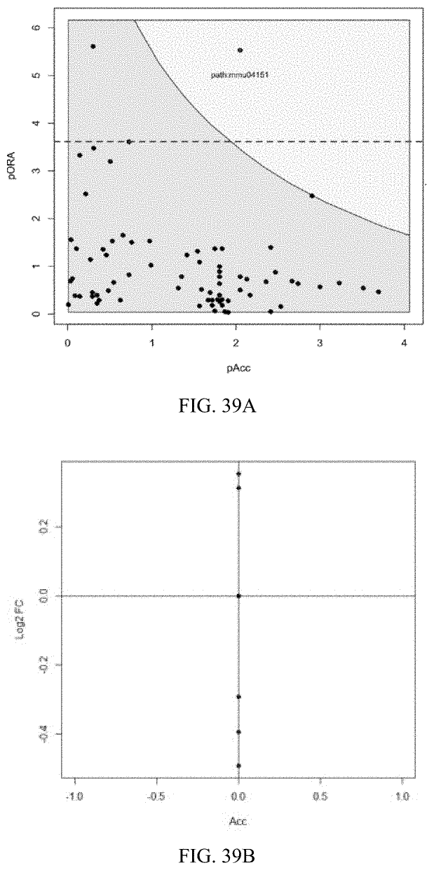

In contrast, male mice displayed more abundant alterations over time. Whereas only one pathway was significantly enriched between young and middle-aged, eight pathways were significantly enriched between young and aged timepoints (FIG. 20C and FIGS. 38 and 39). Of these, PI3K/Akt Signaling was the only pathway that overlapped between the young vs. middle-aged and the young vs. aged comparisons (FIG. 20D). This finding suggests PI3K/Akt Signaling may be a key pathway implicated in aging-induced cartilage degeneration in males, consistent with previous reports demonstrating that alterations in PI3k/Akt Signaling have been previously observed in human OA in comparison to healthy cartilage (Sun, et al., Osteoarthritis Cartilage 28, 400-409 (2020)).

The difference in total normalized perturbation in PI3K/Akt Signaling from young vs. middle-aged to young vs. aged was only 0.126537 (ΔPₙₒᵣₘ=P_{norm,YoungAged}−P_{norm,YoungMiddle-Aged}) (FIG. 20D). Total normalized perturbation is a measure of how much a pathway deviates from physiologic conditions (Tarca, et al., Bioinformatics 25, 75-82 (2009)), with zero being normal physiologic conditions. For these analyses, young mice served as our standard of physiologic conditions (P_{young}=0). This finding suggests that most of the deviation from physiologic conditions is observed from young to middle-aged (FIG. 20B). PI3K/Akt Signaling also represented a point of overlap with other pathways that were significantly enriched according to age. For example, HPV Infection is predominantly driven by PI3K/Akt Signaling, Focal Adhesion, and Jak/STAT Signaling. PI3K/Akt Signaling is of particular interest given it has been implicated in many chondrocyte cellular processes, including metabolism, apoptosis, and inflammation. Cellular senescence, which was another pathway observed in the analysis, is also a downstream effect of PI3K/Akt Signaling (FIG. 20C) (see Sun, et al., 2020, supra).

Further examination of individual proteins associated with PI3K/Akt Signaling enrichment revealed distinctly upregulated or downregulated clusters (FIG. 20E). Upregulated proteins were associated with Metabolic Stress, Mechano-transduction, and Apoptosis, whereas downregulated proteins were associated with Matrix Organization, Cell Proliferation, and Protein Metabolism. Given the fundamental processes revealed by this GO term analysis, these data suggest that PI3K/Akt signaling may be a central regulator for age- and sex-dependent cartilage degeneration (FIG. 20F). Potential age-dependent drivers of this pathway were next identified.

Age-Related Declines in α-Klotho are Associated with Age-Induced Cartilage Degeneration in Male Mice

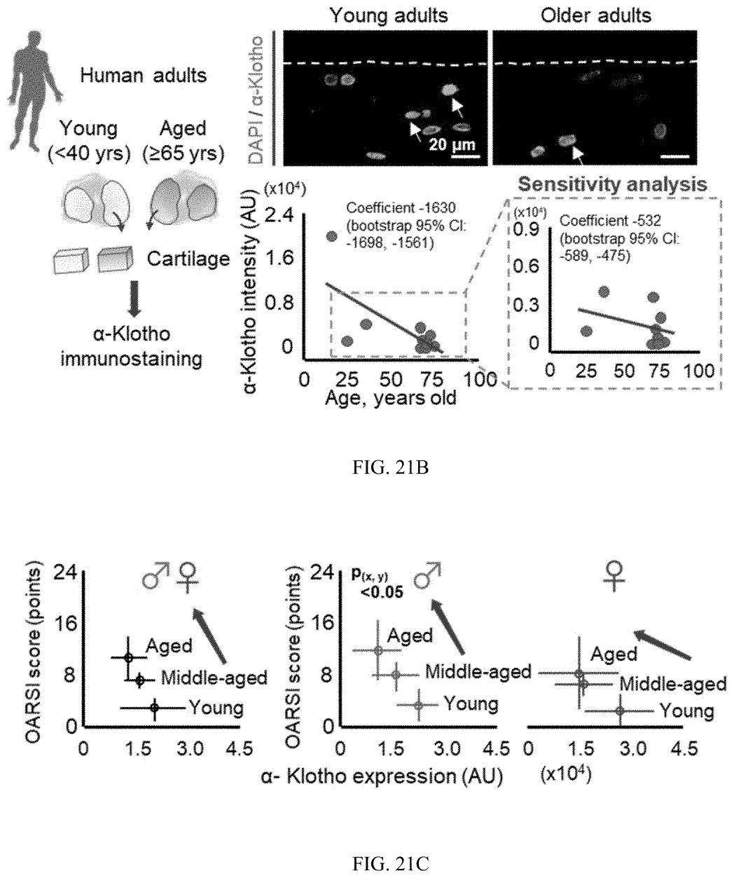

Given the predominance of PBK-Akt signaling dysregulation over time, we probed for candidate proteins that may mediate these changes. Given that Insulin-IGF signaling pathway is a major signaling pathway that reguates cellular aging, one potential candidate that emerged was the longevity protein, α-Klotho (FIG. 20F). α-Klotho serves as a suppressor of aging through a variety of mechanisms related to PI3K-Akt signaling, including inhibition of senescence and autophagy (Bian, et al., Clin Interv Aging 10, 1233-1243 (2015)). In both mouse and human cartilage, α-Klotho was significantly decreased with increasing age (FIG. 21A-B), and was significantly associated with more severe cartilage degeneration in male mice (FIG. 21C). It is hypothesized that age-related declines in α-Klotho may contribute to progressive cartilage degeneration over time.

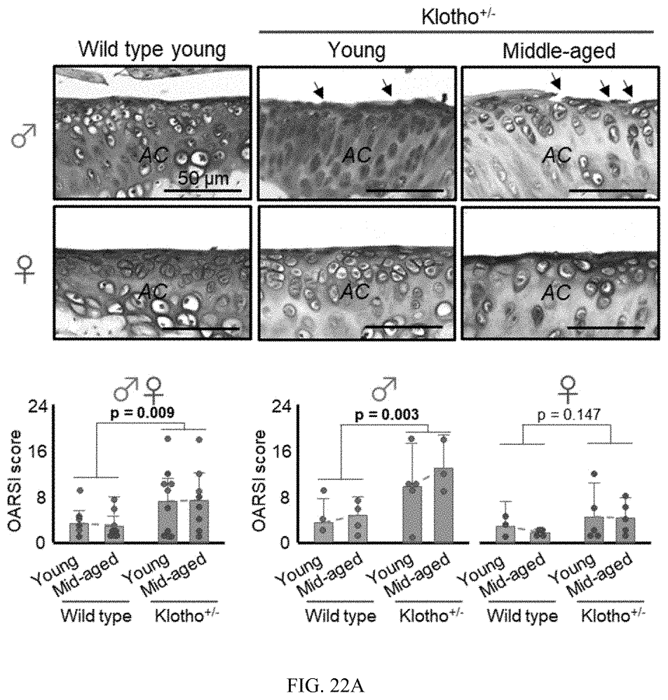

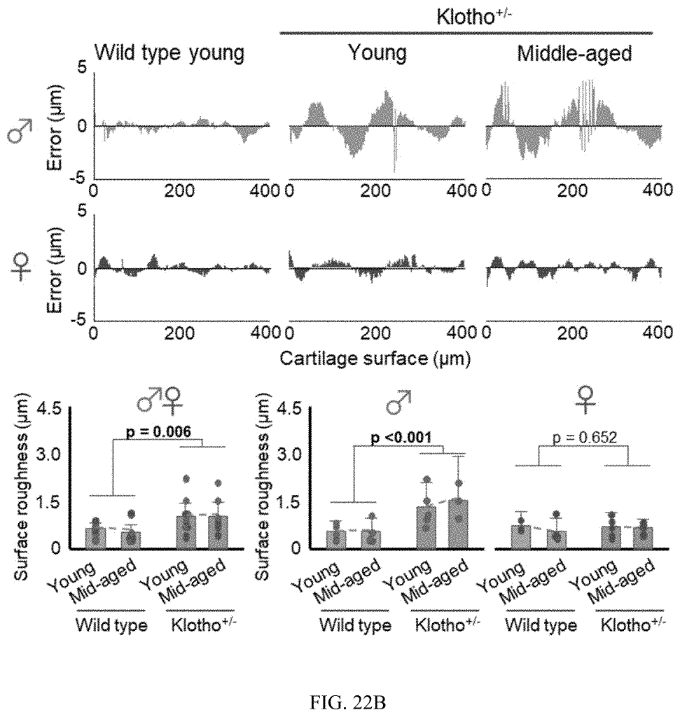

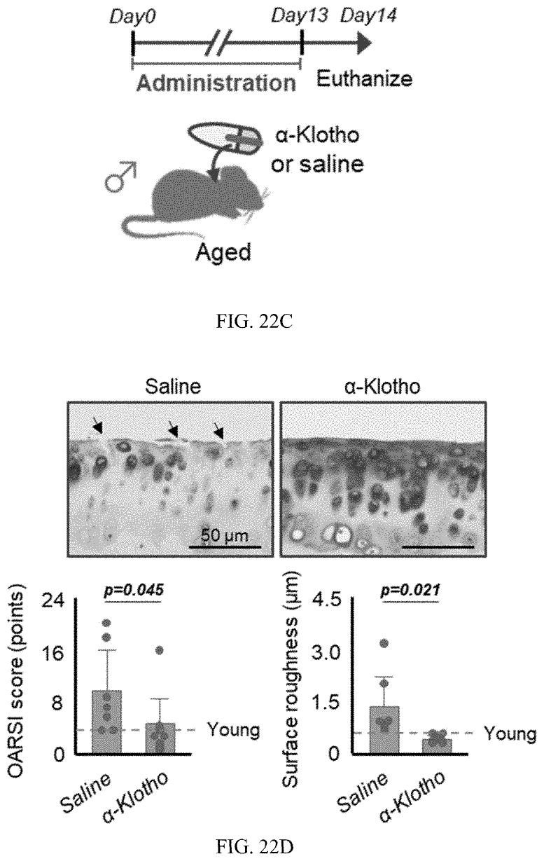

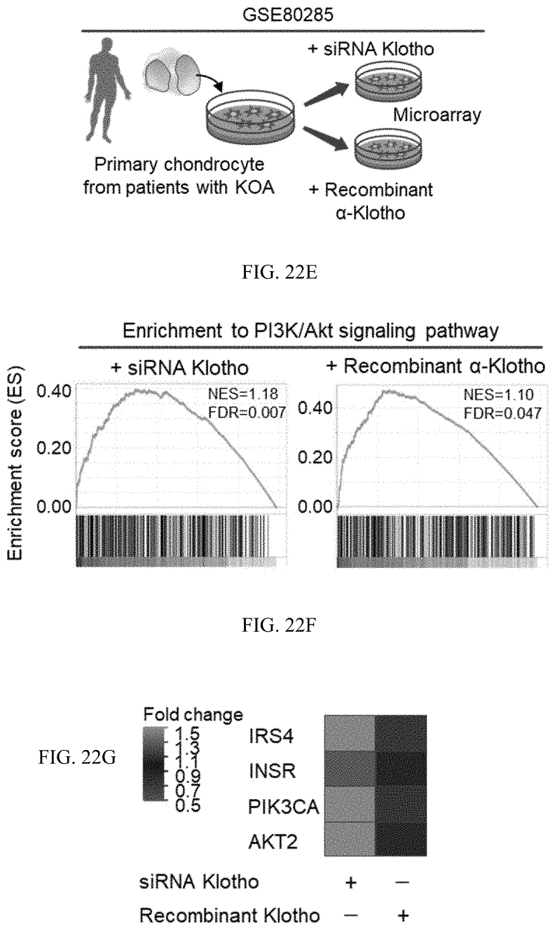

To probe whether α-Klotho plays a direct effect on cartilage health, cartilage integrity was evaluated in mice heterozygously deficient for α-Klotho (Klotho HET). Young and middle-aged Klotho HET male mice displayed significantly accelerated cartilage degradation and increased surface roughness (FIGS. 22A and 22B) consistent with an aged phenotype. Such changes were not observed in female young and middle-aged Klotho HET mice (FIGS. 22A and 22B). These findings suggest that loss of α-Klotho may be a driver of age-related KOA in a sex-dependent manner. Given the limited KOA phenotype in female aged mice, only male mice were used for the remainder of our experiments.

In a subsequent gain-of-function paradigm, it was tested whether α-Klotho protein administration could ameliorate age-related cartilage degeneration. α-Klotho protein was systemically administered to aged mice via osmotic pump (FIG. 22C), and it was found that administration of α-Klotho significantly improved cartilage integrity to levels comparable to young counterparts (FIG. 22D).

The findings presented herein suggest that α-Klotho exerts a protective effect on aging-induced cartilage degeneration. It was next sought to interrogate potential mechanisms underlying the chondroprotective effects of α-Klotho. Although α-Klotho is known to regulate PI3K/Akt signaling (FIG. 20F), evidence supporting a relationship between α-Klotho and PI3K/Akt signaling in articular cartilage is lacking. Thus, archived microarray data (GSE80285) for primary chondrocytes in patients with KOA after treatments of siRNA Klotho or recombinant α-Klotho protein (FIG. 22E) was accessed, and gene set enrichment analysis revealed that both siRNA Klotho and α-Klotho protein supplementations significantly changed PI3K/Akt signaling (FIG. 22F). When individual genes were probed, key elements of PI3K/Akt signaling, including PIK3CA and AKT2, were upregulated by siRNA Klotho but downregulated by α-Klotho protein supplementation (FIG. 22G). In addition, α-Klotho downregulated key elements of Insulin signaling including IRS4 and INSR. These in vitro results suggest that α-Klotho inhibits Insulin/PI3K/Akt signaling in chondrocytes from patients with KOA. Collectively, these in vivo and in vitro findings suggest that Insulin/PI3K/Akt signaling may mediate the detrimental effects of α-Klotho decline in articular cartilage.

Age-Related Decline in α-Klotho is Associated with Alterations in Nuclear Morphology in Mice and Humans

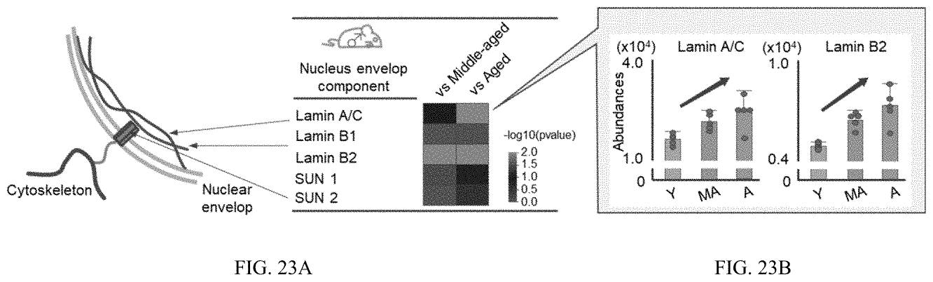

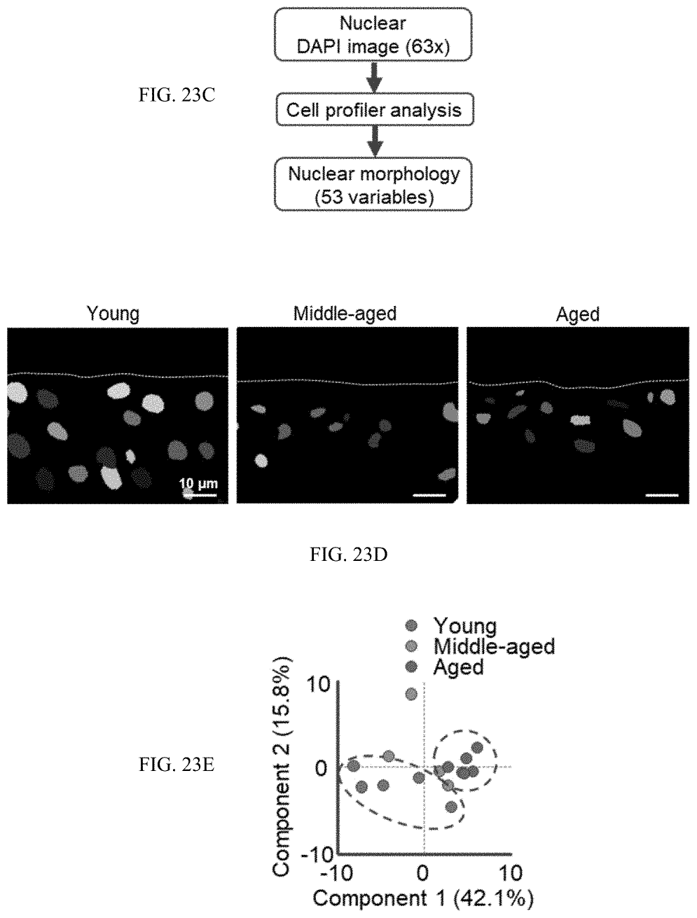

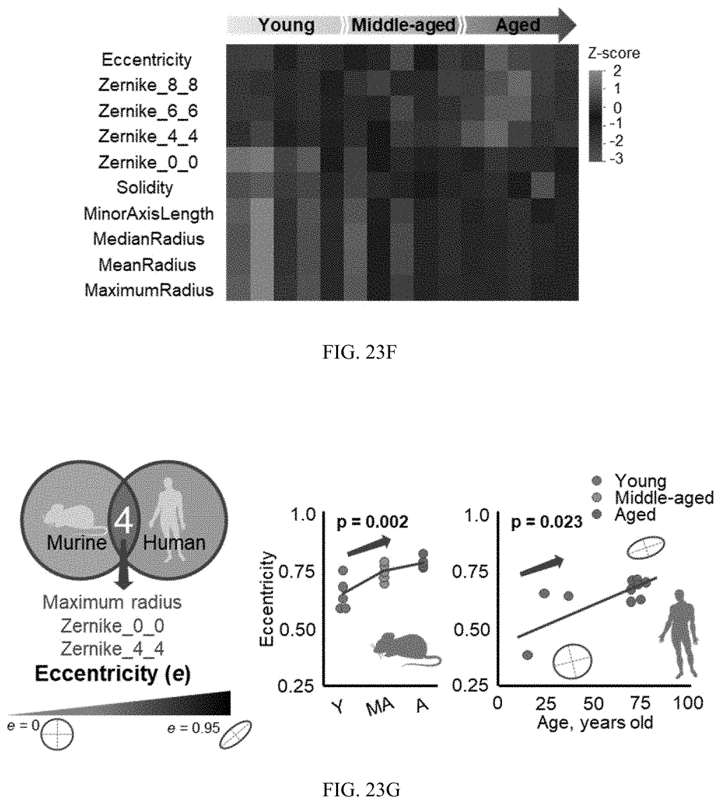

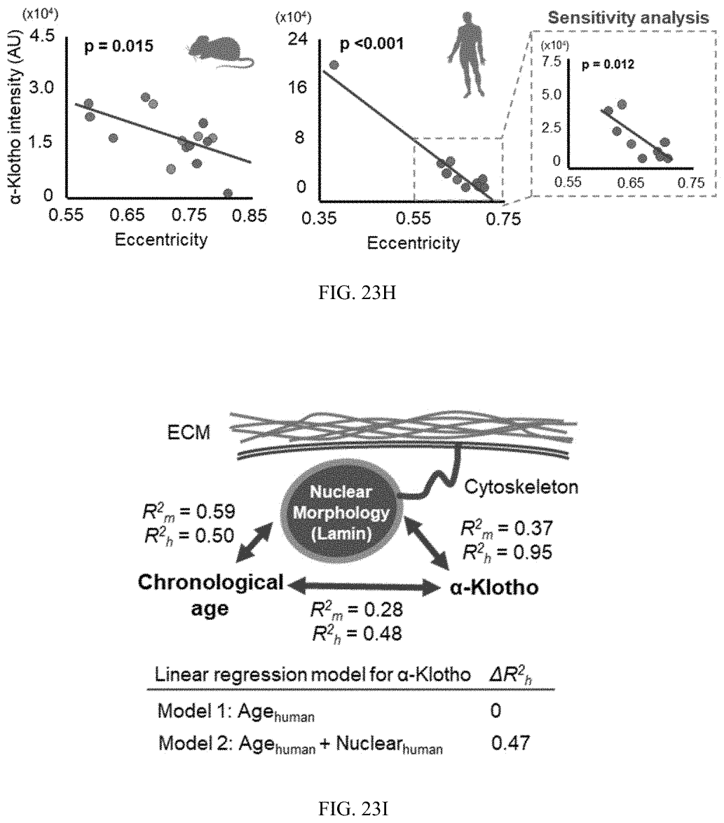

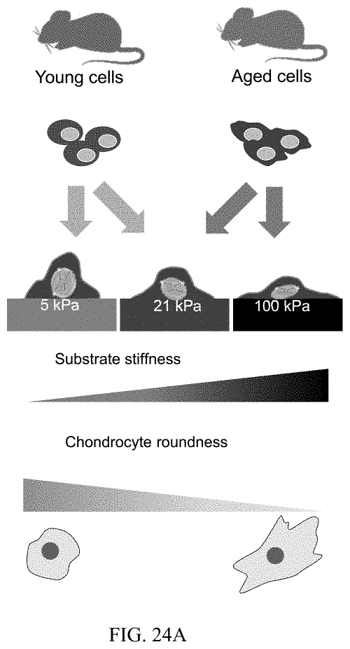

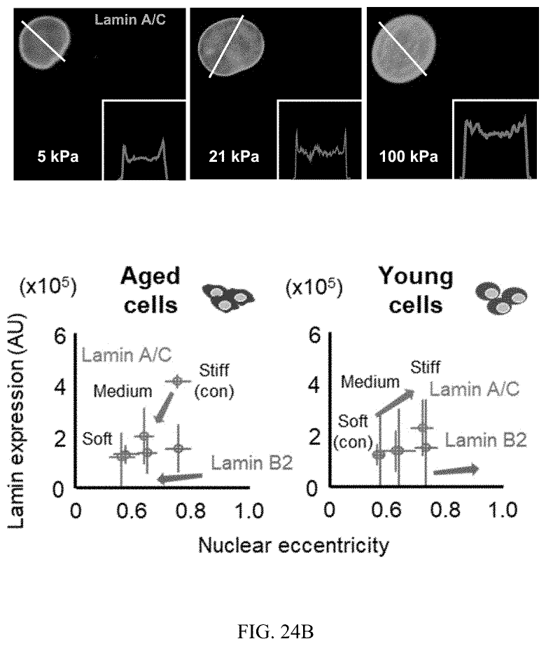

What drives decline in α-Klotho protein levels over time? Whereas numerous studies in rodents and humans have demonstrated that loss of α-Klotho promotes an aged phenotype in tissues throughout the organism, little is known about the molecular mechanisms driving this decline. Gene expression can be altered as a result of age-related changes in the cellular microenvironment through mechanotransductive signaling. Cells sense and respond to biophysical niche signals through cytoskeleton-mediated alterations in nuclear morphology (Jahed, et al., International review of cell and molecular biology 310, 171-220 (2014)). Indeed, nuclear envelope dysfunction leads to chromatin remodeling and has been tightly linked to cellular aging (Martins, et al., Aging cell 19, e13143 (2020)). The nuclear envelope is primarily composed of nuclear lamina (i.e., lamin A/C and lamin B) and a double membrane through the linker of the nucleoskeleton and cytoskeleton (LINC) complex such as Sun proteins. Lamin A/C is a well-known regulator of nuclear integrity and mechanotransduction (Lammerding, et al., J Clin Invest 113, 370-378 (2004); Lammerding, et al., J Biol Chem 281, 25768-25780 (2006)). With this in mind, the mass spectrometry data was revisited to probe for age-related changes in nuclear envelope elements (FIG. 23A). Indeed, there was increased expression of nuclear envelope elements including lamin A/C and lamin B2 in aged mice compared to young counterparts (FIG. 23B). These findings indicate that reorganization of the nuclear envelope and associated mechanotransductive signaling alteration may be related to chondrocyte aging.

To further support these findings, chondrocyte nuclear morphology was quantified according to 53 different variables using Cell Profiler software (Carpenter, 2006, supra) (FIGS. 23C and 23D). Principal component analysis (PCA) of nuclear morphological features revealed clear segregation of nuclei from young and aged chondrocytes (FIG. 23E). Of the top 10 variables contributing to the first principal component (FIG. 23F), we identified four common characteristics changed with aging when comparing murine and human studies, including maximum radius, Zernike shapes, and eccentricity (FIG. 23G). Notably, increased nuclear eccentricity (i.e., less spherical nucleus) was associated with decreased α-Klotho expression in both murine and human samples (FIG. 23H), suggesting a relationship between age-related changes in nuclear morphology and declines in α-Klotho expression (FIG. 23I). These results suggest that decline in α-Klotho levels may be attributed, at least in part, to altered nuclear morphology (FIG. 23I).

### Substrate Stiffness Regulates α-Klotho Expression in Chondrocytes Regardless of Age

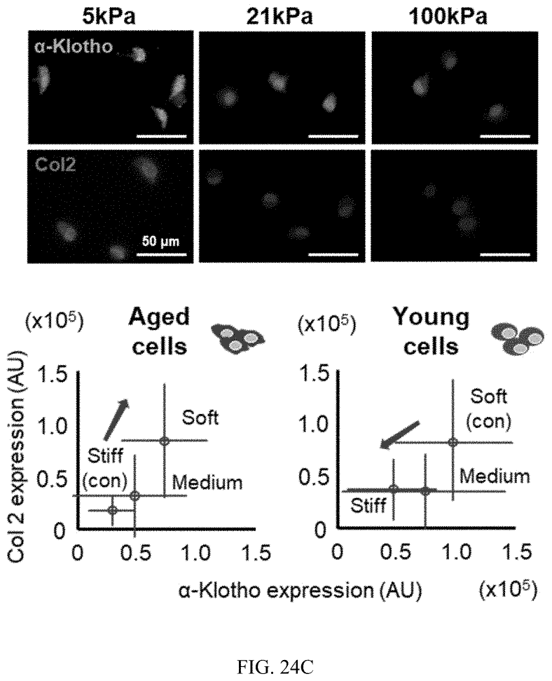

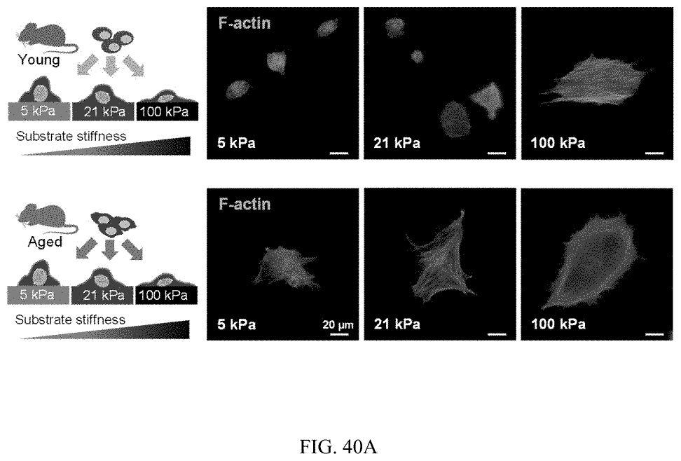

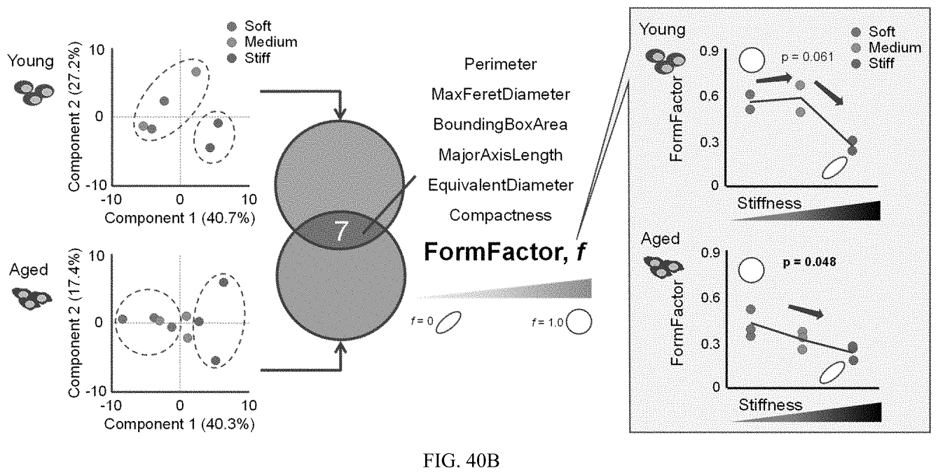

Nuclear morphology is mechanically coupled to the surrounding microenvironment where cytoskeletal elements regulate nuclear shape according to ECMstiffness (Swift, et al., Science 341, 1240104 (2013); Buxboim, et al., Curr Biol 24, 1909-1917 (2014)). As cartilage stiffness in aged mice and humans is 2-3 times higher compared to young counterparts (Stolz, et al., Nat Nanotechnol 4, 186-192 (2009)), a stiff ECM may drive young chondrocytes towards an aged phenotype and a soft ECM would restore a more youthful profile. To test this hypothesis, young or aged primary mouse chondrocytes were seeded onto polyacrylamide gels engineered to mimic a physiological range of knee cartilage ECM stiffnesses (5 kPa, 21 kPa, and 100 kPa) (FIG. 24A). This stiffness range was selected to mimic a physiologically-relevant ECM stiffnesses in young (5-30 kPa) and aged (50-100 kPa) murine and human knee cartilage, and this range of stiffness can induce morphological changes in chondrocytes (FIG. 40).

Aged chondrocytes cultured on soft substrates displayed a more youthful phenotype when compared to cells cultured on stiffer substrates, as evidenced by lower nuclear lamin A/C expression (FIG. 24B), a more spherical nuclear shape, as well as increased type II collagen and α-Klotho expression (FIG. 24C). On the other hand, young chondrocytes cultured on stiff substrates developed an aged phenotype, specifically increased lamin A/C expression, a less spherical nuclear shape, and reduced type II collagen and α-Klotho expression (FIG. 24C). In contrast, lamin B2 expression was not sensitive to substrate stiffness (FIG. 24B). Stiffer substrate increased stress fiber formation which was accompanied with chondrocytes morphological alterations (FIG. 40). Collectively, these findings illustrate a clear relationship between ECM biophysical properties, nuclear stiffness, α-Klotho expression, and chondrogenecity regardless of age.

### Reduced Matrix Stiffness Improves α-Klotho Expression in Aged Mice

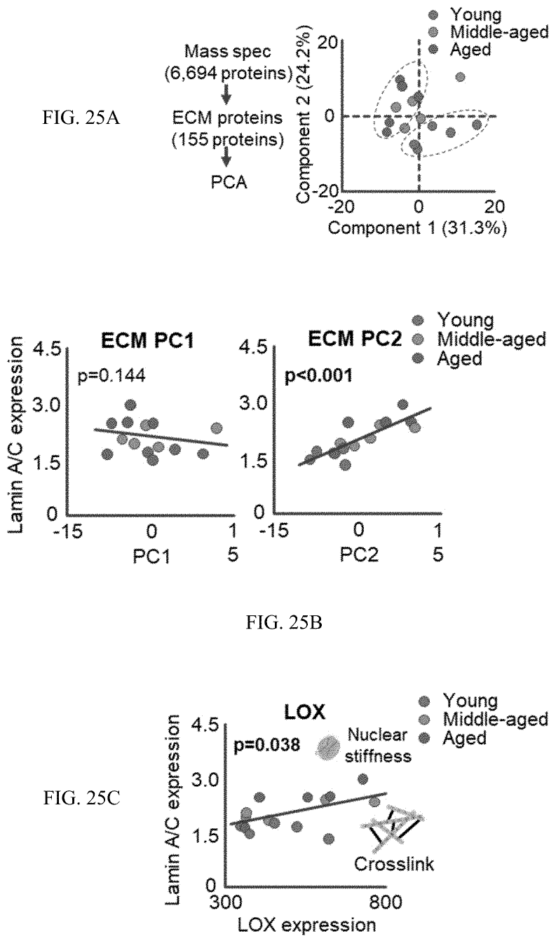

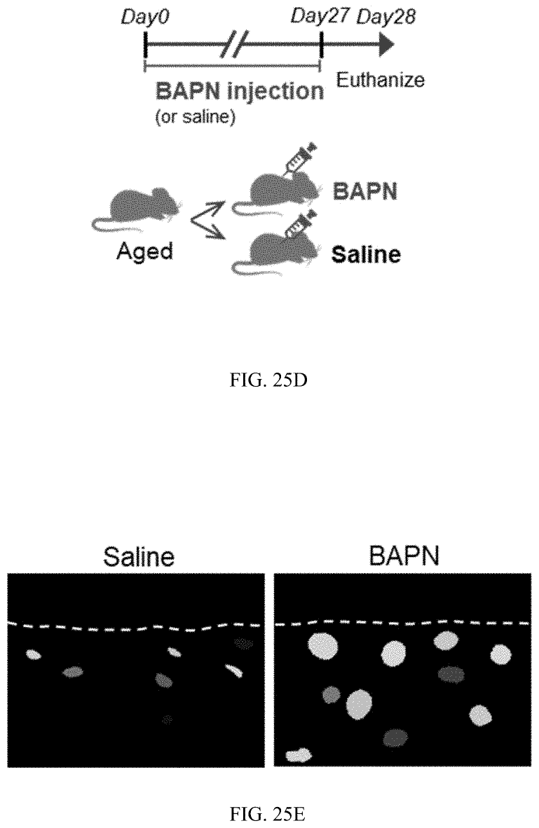

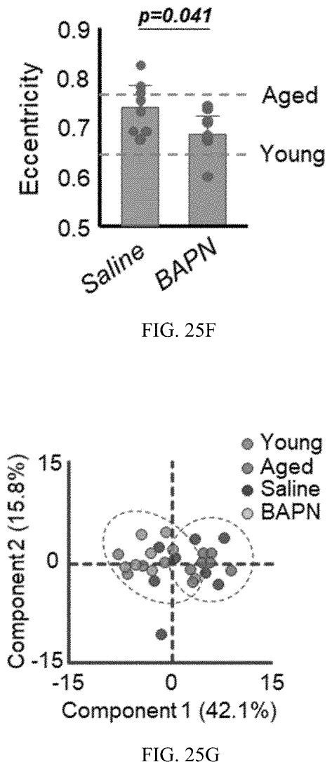

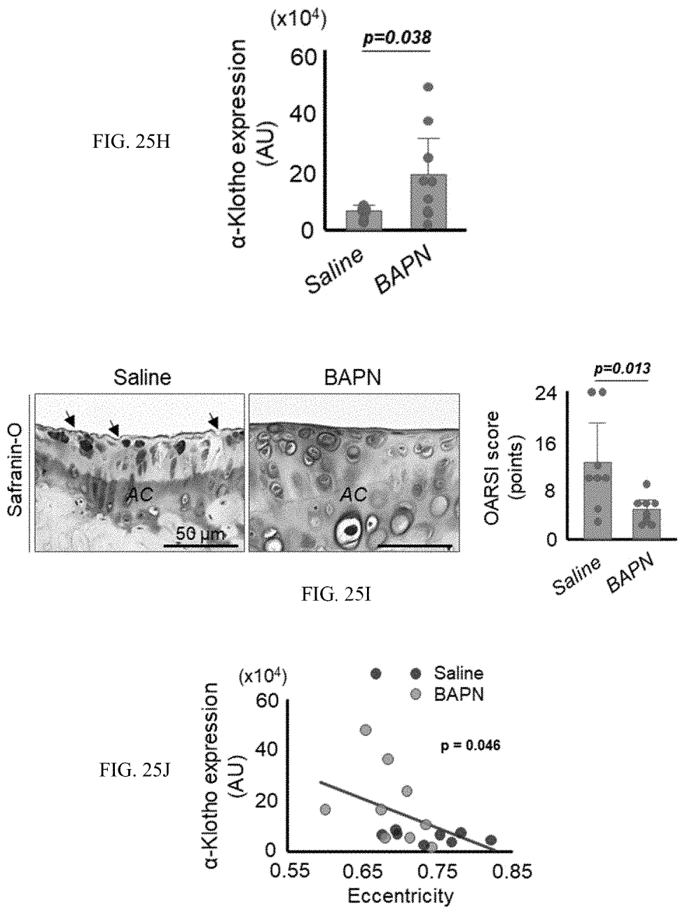

To identify ECM proteins that may contribute to the increased nuclear stiffness (i.e., lamin A/C) in vivo, the mass spectrometry data was assessed. Pathway analysis revealed enrichment in ECM related pathways including Focal Adhesion, ECM Receptor Interaction, Hippo Signaling, and Proteoglycans in Cancer (FIG. 20C). Accordingly, PCA revealed that aged cartilage displayed altered expression profile in 155 ECM-related proteins detected in the mass spectrometry data (FIG. 25A). Lysyl oxidase (LOX) emerged within the top 20 proteins contributing to principal component 2, which was significantly correlated with lamin A/C (FIG. 25B). This is particularly of interest given that LOX is one of the major enzymes that induce collagen crosslinking and contribute to pathogenesis of PTOA through increased ECM stiffness (Kim, et al., Proc Natl Acad Sci USA 112, 9424-9429 (2015)). A significant positive correlation was observed between the LOX and Lamin A/C (FIG. 25C) and it was hypothesized that increased nuclear stiffness and subsequent declines in α-Klotho in aged cartilage may be regulated, at least in part, by LOX.

To test this hypothesis and confirm the physiological relevance of in vitro findings, β-aminopropionitrile (BAPN), a known inhibitor of LOX, was administered to aged mice daily for four weeks (FIG. 25D). Histological analysis revealed that cartilage from aged animals treated with BAPN displayed a more spherical nuclear morphology, with decreased nuclear eccentricity, thereby mimicking the chondrocyte morphology of young mice (FIGS. 25E and 25F). PCA analysis further confirmed a more youthful phenotype of chondrocytes from aged mice treated with BAPN clustered with young nuclei, and BAPN-treated aged nuclei displayed decreased nuclear eccentricity, as determined by PCA (FIGS. 25F-25G). Finally, consistent with in vitro studies, modulation of matrix stiffness in vivo significantly increased α-Klotho levels in aged chondrocytes (FIG. 25H) and improved cartilage integrity (FIGS. 25I-25J).

This examples shows that progressive disruption of the intracellular signaling transduction pathway PI3K/Akt signaling was associated with the onset of age-related KOA in a sex-dependent manner. In search of upstream candidates that may drive these changes, it was discovered that both age-related and genetically-induced declines in α-Klotho, a suppressor of PI3K/Akt signaling, drove cartilage degeneration in mice. This age-related decrease in chondrocyte α-Klotho expression was corroborated in young and aged human cartilage. Chondrocyte α-Klotho expression was highly responsive to biophysical characteristics of the ECM. As is observed with increasing age, increased matrix stiffness drove decreased α-Klotho expression. Conversely, decreased matrix stiffness in vivo increased α-Klotho expression and improved chondrogenecity.

### PI3K/Akt Signaling: A Key in Elucidating Age-Induced Cartilage Degeneration in a Sex-Dependent Manner

Despite the fact that there are many studies characterizing tissue-level changes in cartilage with aging, there is a paucity of work that has thoroughly examined cartilage integrity according to sex. This is a critical shortcoming of the existing literature given that post-menopausal women typically present with more severe KOA. To address this gap, the trajectory of cartilage degeneration over time and according to sex was analyzed using well-established histological analyses. It was found that age-related cartilage degeneration was more severe in male mice, findings that are inconsistent with the clinical evidence of an increased prevalence of KOA in women. This disconnect may result from the fact that the aged female mice used in this study were non-menopausal, as evidenced by maintained serum estrogen levels across aging. Estrogen affects key signaling molecules in several distinct canonical and non-canonical estrogen signaling pathways, such as PI3K/Akt and PKC/MAPK signaling (Roman Blas, et al., Arthritis Res Ther 11, 241 (2009)). Whereas the beneficial role of systemic estrogen administration in OA development has been investigated in vivo, the underlying mechanism remains unknown. This data highlight the need for mechanistic studies into the effects of menopause on KOA.

This study confirmed that age-related KOA was accompanied by disruption of the PI3K/Akt signaling cascade. PI3K/Akt signaling disruption contributes to imbalance of ECM production and destruction, and this pathway is dysregulated in the context of human OA. The important role of this pathway in OA development is further evidenced by in vivo studies showing that modulating PI3K/Akt signaling attenuated pathological cartilage changes in PTOA knees. Although little is known about the pathogenic contribution of this pathway in age-related KOA, involvement of PI3K/Akt signaling is generally in line with previous finding of age-related disruption in insulin-like growth factor 1 signaling in human chondrocytes. It is noteworthy that alterations in PI3K/Akt signaling were already evident in middle-aged mice, coinciding with the onset of cartilage structural abnormalities in humans. Early disruption of PBK-Akt signaling is supported by a previous microarray analysis showing upregulated inflammation-related genes and down-regulated ECM-related genes in middle-aged mice (Loeser, et al., Arthritis Rheum 64, 705-717 (2012)), both of which are regulated by PI3K-Akt signaling (Sun et al., 2020, supra). The observation that only PI3K-Akt signaling was significantly changed in middle-aged mice suggests that this pathway may be an early driver of OA pathogenesis.

### α-Klotho as a Potential Mediator Between Alterations in the ECM and Cartilage Integrity

In search of upstream candidates that may regulate PI3K/Akt signaling, it was discovered that diminished α-Klotho, a longevity protein, compounded cartilage degeneration with aging, which reinforces previous in vitro and in vivo studies showing that α-Klotho overexpression counteracts chondrocyte dysfunction and cartilage degeneration (Chuchana, et al., Aging 10, 1442-1453 (2018); Gu, et al., Am J Transl Res 11, 7338-7350 (2019)). α-Klotho inhibits insulin growth factor receptor-mediated PI3K/Akt signaling and subsequently enhances FoxO, alleviating effects of oxidative stress (Yamamoto, et al., J Biol Chem 280, 38029-38034 (2005). FoxO is markedly reduced with aging both in murine and human cartilage (38), which increases susceptibility to oxidative stress induced chondrocyte death and impairs cartilage integrity (Akasaki, et al., Arthritis Rheumatol 66, 3349-3358 (2014); Matsuzaki, et al., Science translational medicine 10, 428 (2018). Taken together, these results suggest that age-related cartilage degeneration is attributed, at least in part, to reduced protection from oxidative stress that is driven by declines in α-Klotho.

Whereas age-related declines in α-Klotho have been linked to the onset of an aged tissue phenotype in many organ systems, the understanding of the mechanisms driving these declines is lacking. The ECM plays a dynamic role in regulating cartilage homeostasis and undergoes extensive remodeling with increasing age, including a decrease in compliance. It is well established that increased matrix stiffness disrupts chondrocyte functionality via mechanotransductive pathways, leading to increased cellular senenscence, and these mechanical cues from the ECM signal alterations in gene expression. In this example, matrix stiffness was identified as a novel regulator of α-Klotho expression. Specifically, increased matrix stiffness decreased α-Klotho expression and drove chondrocytes to an aged phenotype in vitro. Conversely, decreasing matrix stiffness by inhibiting LOX activity improved α-Klotho expression, leading to improved cartilage integrity. This finding is consistent with reports showing that reduced LOX-mediated collagen cross-linking improved cartilage integrity in PTOA (Kim, et al., 2015, supra), and showed LOX-mediated collagen cross-linking to be mediated through mechanotransduction of the RhoA/ROCK axis. This is in contrast to what is being observed with α-Klotho and highlights distinct mechanisms between age-related and PTOA models.

One prominent feature linking age-related changes in ECM stiffness to altered α-Klotho expression is nuclear morphological alteration. Remodeling in nuclear morphology are closely associated with modified gene expression and protein synthesis. In this study, a strong relationships was observed between high nuclear deformation and α-Klotho decline in both murine and human samples. Notably, the higher nuclear deformation seen in aged cartilage was recapitulated by a stiff microenvironment. These nuclear morphological alterations are attributed to increased Lamin A/C, a primary mediator of nuclear integrity in response to matrix elasticity compared to Lamin B. Altered expression of Lamin A/C directly influence chromatin dynamics, leading to altered gene expression and protein synthesis (Bronshtein, et al., Nat Commun 6, 8044 (2015)). The findings herein indicate that age-related alterations in ECM biophysical properties change nuclear integrity, ultimately inhibiting α-Klotho expression.

### Example 4—Age-Related Increase in Matrix Stiffness Downregulates α-Klotho in Chondrocytes and Induces Cartilage Degeneration

The lack of disease-modifying treatments for KOA is attributed, in part, to an incomplete understanding of the molecular mechanisms driving disease development. The pathologic cascade currently associated with KOA includes inflammation, impaired autophagy, and cellular senescence, which are hallmarks of aging. Therefore, identification of therapeutic targets that can rejuvenate aged cartilage represents a promising research direction for the treatment of age-related KOA. The longevity protein, α-Klotho, plays an important role in the attenuation of cellular senescence. Studies have shown α-Klotho decreases with aging, whereas α-Klotho supplementation counteracts age-induced cartilage degeneration. However, little is known about the underlying mechanisms driving declines in a-Klotho expression in cartilage. Such knowledge may aid the development of therapeutics that promote a regenerative microenvironment for chondrocytes and maintain cartilage integrity during aging. In this example, the mechanisms leading to age-related declines in α-Klotho in cartilage were examined. The ECM plays a dynamic role in directing chondrocyte phenotype and cartilage homeostasis, and undergoes extensive biophysical remodeling characterized by decreased compliance (i.e., increased ECM stiffness) during aging. This increased stiffness is due, in part, to a loss of proteoglycans and increased collagen cross-linking. It was hypothesized that an aged stiff microenvironment drives a decline in chondrocyte expression of α-Klotho, thereby impairing cartilage integrity, but a youthful soft microenvironment increases α-Klotho expression, thereby promoting a more healthy phenotype.

### Methods

First, the trajectory of α-Klotho expression and cartilage integrity was with aging was evaluated using histology, isolating knee cartilage from young (4-6 mo), middle-aged, (10-12 mo), and aged (21-24 mo) male and female C57/BL6 mice, as well as young and middle-aged α-Klotho+/− mice. Cartilage from young, middle-aged, and aged mice was microdissected for LC-MS/MS mass spectrometry proteomics to further explore the mechanisms of natural aging on knee cartilage, performing gene set enrichment analysis (GSEA) to evaluate pathways of interest. Next, polyacrylamide gels were engineered within a physiological range of cartilage ECM stiffness (5 kPa, 20 kPa, and 100 kPa) and seeded young or aged primary chondrocytes onto the gel surfaces. After 3 days in culture: (1) chondrogenicity (type II collagen expression), and (2) α-Klotho expression was evaluated using immunofluorescent staining. To explore the role of ECM stiffness in vivo, β-aminopropionitrile (BAPN), an inhibitor of lysyl oxidase (LOX) activity, was administered daily for four weeks. Both (1) cartilage integrity and (2) α-Klotho expression in chondrocytes was evaluated by histology and immunofluorescence. Finally, to probe the potential mechanotransductive mechanisms associated with the effect of biophysical ECM properties on α-Klotho expression, 53 features of chondrocyte nuclear morphology were extracted across experimental groups using Cell Profiler software and performed principal component analysis (PCA) with nuclear morphology features as input variables.

### Results

As expected, histological studies confirmed progressive cartilage degeneration with aging starting from middle-aged mice, including reduced cellularity and decreased α-Klotho expression. Notably, histological evidence of KOA with aging was recapitulated in young and middle aged Klotho+/− mice, suggesting that an age-related loss of α-Klotho may contribute to the onset of cartilage degeneration. The molecular changes observed in middle-aged mice were supported by GSEA, with three pathways signficantly upregulated at middle-age, including: (1) Cell Adhesion, (2) Extracellular Matrix, and (3) Signal Transduction Activity or Receptor Binding. Therefore, it was determined whether the ECM influences α-Klotho expression and chondrogenicity in isolated chondrocytes. Independent of age, chondrocytes seeded on stiff substrates displayed an “aged phenotype”, as indicated by reduced type II collagen and α-Klotho expression. Conversely, chondrocytes seeded on soft substrates displayed a more “youthful phenotype”, as indicated by increased type II collagen and α-Klotho expression. The changes observed when cells were cultured on soft substrates were consistent with a more youthful nuclear morphology, and specifically, decreased eccentricity (i.e., more spherical nucleus). These in vitro findings were further supported by in vivo histological analysis, which revealed aged cartilage had less spherical nuclei. Moreover, the decreased sphericity was associated with decreased α-Klotho expression. Inspired by the in vitro findings, it was tested whether modulation of biophysical ECM properties in vivo could exert similar effects on α-Klotho expression and cartilage integrity. BAPN injections increased α-Klotho expression and improved cartilage integrity in aged mice. Interestingly, histological findings revealed that BAPN administration induced a more spherical nuclear morphology, mimicking young chondrocytes. Notably, PCA of nuclear morphological features revealed BAPN-treated nuclei clustered with young nuclei but segregated from aged control nuclei. Specifically, BAPN-treated aged nuclei displayed decreased nuclear eccentricity, which was again inversely correlated with α-Klotho levels.

Evidence of the accelerated cartilage degeneration in Klotho+/− mice suggests age-related declines in α-Klotho may contribute to the onset of OA. In vitro and in vivo experiments further indicated that a stiffer microenvironment, as is observed with aging, decreased α-Klotho and type II collagen expression. On the other hand, a softer microenvironment restored aged chondrocytes towards a more youthful phenotype with increased α-Klotho expression. These findings also suggest that the rejuvenating effects of a softer microenvironment may be partially attributed to altered mechanotransductive signaling, as is suggested by alterations in nuclear morphology.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a mammal having osteoarthritis, wherein said method comprises administering an α-Klotho polypeptide or nucleic acid encoding said α-Klotho polypeptide to a mammal identified as having osteoarthritis.

2. The method of claim 1, wherein said mammal is a human.

3. The method of claim 1, wherein said method comprises administering said α-Klotho polypeptide to said mammal.

4. The method of claim 1, wherein said method comprises administering said nucleic acid to said mammal.

5. The method of claim 4, wherein said nucleic acid is a viral vector.

6. The method of claim 4, wherein said nucleic acid is naked DNA.

7. The method of claim 1, wherein said osteoarthritis is present in a knee of said mammal.

8. The method of claim 1, wherein said administering comprises an intra-articular injection.

9. The method of claim 1, wherein said administering comprises an intravenous injection.

10. A method for improving cartilage integrity within a mammal having osteoarthritis, wherein said method comprises administering an α-Klotho polypeptide or nucleic acid encoding said α-Klotho polypeptide to a mammal identified as having osteoarthritis.

11. The method of claim 10, wherein said mammal is a human.

12. The method of claim 10, wherein said method comprises administering said α-Klotho polypeptide to said mammal.

13. The method of claim 10, wherein said method comprises administering said nucleic acid to said mammal.

14. The method of claim 13, wherein said nucleic acid is a viral vector.

15. The method of claim 13, wherein said nucleic acid is naked DNA.

16. The method of claim 10, wherein said osteoarthritis is present in a knee of said mammal.

17. The method of claim 10, wherein said administering comprises an intra-articular injection.

18. The method of claim 10, wherein said administering comprises an intravenous injection.

19. A method for reducing cartilage degeneration within a mammal having osteoarthritis, wherein said method comprises administering an α-Klotho polypeptide or nucleic acid encoding said α-Klotho polypeptide to a mammal identified as having osteoarthritis.

20. The method of claim 19, wherein said mammal is a human.

21. The method of claim 19, wherein said method comprises administering said α-Klotho polypeptide to said mammal.

22. The method of claim 19, wherein said method comprises administering said nucleic acid to said mammal.

23. The method of claim 22, wherein said nucleic acid is a viral vector.

24. The method of claim 22, wherein said nucleic acid is naked DNA.

25. The method of claim 19, wherein said osteoarthritis is present in a knee of said mammal.

26. The method of claim 19, wherein said administering comprises an intra-articular injection.

27. The method of claim 19, wherein said administering comprises an intravenous injection.


SEQUENCE LISTING (2 sequences)

SEQ ID NO: 1 - protein, 1012 aa, Homo sapiens
MPASAPPRRPRPPPPSLSLLLVLLGLGGRRLRAEPGDGAQTWARFSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLLAFIAFLFFASIISLSLIFYYSKKGRRSYK

SEQ ID NO: 2 - DNA, 5012 bp, Homo sapiens
cgcgcagcatgcccgccagcgccccgccgcgccgcccgcggccgccgccgccgtcgctgtcgctgctgctggtgctgctgggcctgggcggccgccgcctgcgtgcggagccgggcgacggcgcgcagacctgggcccgtttctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggatacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttccctgtgactttgcttggggagttgttgacaactacattcaagtagataccactctgtctcagtttaccgacctgaatgtttacctgtgggatgtccaccacagtaaaaggcttattaaagtggatggggttgtgaccaagaagaggaaatcctactgtgttgactttgctgccatccagccccagatcgctttactccaggaaatgcacgttacacattttcgcttctccctggactgggccctgattctccctctgggtaaccagtcccaggtgaaccacaccatcctgcagtactatcgctgcatggccagcgagcttgtccgtgtcaacatcaccccagtggtggccctgtggcagcctatggccccgaaccaaggactgccgcgcctcctggccaggcagggcgcctgggagaacccctacactgccctggcctttgcagagtatgcccgactgtgctttcaagagctcggccatcacgtcaagctttggataacgatgaatgagccgtatacaaggaatatgacatacagtgctggccacaaccttctgaaggcccatgccctggcttggcatgtgtacaatgaaaagtttaggcatgctcagaatgggaaaatatccatagccttgcaggctgattggatagaacctgcctgccctttctcccaaaaggacaaagaggtggctgagagagttttggaatttgacattggctggctggctgagcccattttcggctctggagattatccatgggtgatgagggactggctgaaccaaagaaacaattttcttcttccttatttcactgaagatgaaaaaaagctaatccagggtacctttgactttttggctttaagccattataccaccatccttgtagactcagaaaaagaagatccaataaaatacaatgattacctagaagtgcaagaaatgaccgacatcacgtggctcaactcccccagtcaggtggcggtagtgccctgggggttgcgcaaagtgctgaactggctgaagttcaagtacggagacctccccatgtacataatatccaatggaatcgatgacgggctgcatgctgaggacgaccagctgagggtgtattatatgcagaattacataaacgaagctctcaaagcccacatactggatggtatcaatctttgcggatactttgcttattcgtttaacgaccgcacagctccgaggtttggcctctatcgttatgctgcagatcagtttgagcccaaggcatccatgaaacattacaggaaaattattgacagcaatggtttcccgggcccagaaactctggaaagattttgtccagaagaattcaccgtgtgtactgagtgcagtttttttcacacccgaaagtctttactggctttcatagcttttctattttttgcttctattatttctctctcccttatattttactactcgaagaaaggcagaagaagttacaaatagttctgaacatttttctattcattcattttgaaataattatgcagacacatcagctgttaaccatttgcacctctaagtgttgtgaaactgtaaatttcatacatttgacttctagaaaacatttttgtggcttatgacagaggttttgaaatgggcataggtgatcgtaaaatattgaataatgcgaatagtgcctgaatttgttctctttttgggtgattaaaaaactgacaggcactataatttctgtaacacactaacaaaagcatgaaaaataggaaccacaccaatgcaacatttgtgcagaaatttgaatgacaagattaggaatattttcttctgcacccacttctaaatttaatgtttttctggaagtagtaattgcaagagttcgaatagaaagttatgtaccaagtaaccatttctcagctgccataataatgcctagtggcttcccctctgtcaaatctagtttcctatggaaaagaagatggcagatacaggagagacgacagagggtcctaggctggaatgttcctttcgaaagcaatgcttctatcaaatactagtattaatttatgtatctggttaatgacatacttggagagcaaattatggaaatgtgtattttatatgatttttgaggtcctgtctaaaccctgtgtccctgagggatctgtctcactggcatcttgttgagggccttgcacataggaaacttttgataagtatctgcggaaaaacaaacatgaatcctgtgatattgggctcttcaggaagcataaagcaattgtgaaatacagtataccgcagtggctctaggtggaggaaaggaggaaaaagtgcttattatgtgcaacattatgattaatctgattatacaccatttttgagcagatcttggaatgaatgacatgacctttccctagagaataaggatgaaataatcactcattctatgaacagtgacactactttctattctttagctgtactgtaatttctttgagttgatagttttacaaattcttaataggttcaaaagcaatctggtctgaataacactggatttgtttctgtgatctctgaggtctattttatgtttttgctgctacttctgtggaagtagctttgaactagttttactttgaactttcacgctgaaacatgctagtgatatctagaaagggctaattaggtctcatcctttaatgccccttaaataagtcttgctgattttcagacagggaagtctctctattacactggagctgttttatagataagtcaatattgtatcaggcaagataaaccaatgtcataacaggcattgccaacctcactgacacagggtcatagtgtataataatatactgtactatataatatatcatctttagaggtatgattttttcatgaaagataagcttttggtaatattcattttaaagtggacttattaaaattggatgctagagaatcaagtttattttatgtatatatttttctgattataagagtaatatatgttcattgtaaaaatttttaaaacacagaaactatatgcaaagaaaaaataaaaattatctataatctcagaacccagaaatagccactattaacatttcctacgtattttattttacatagatcatattgtatatagttagtatctttattaatttttattatgaaactttcctttgtcattattagtcttcaaaagcatgatttttaatagttgttgagtattccaccacaggaatgtatcacaacttaaccgttcccgtttgttagactagtttcttattaatgttgatgaatgttgtttaaaaataattttgttgctacatttactttaatttccttgactgtaaagagaagtaattttgctccttgataaagtattatattaataataaatctgcctgcaactttttgccttctttcataatcataaaaaaa